(19) United States
(12) Patent Application Publication
(10) Pub. No.: US 2003/0130797 A1
(43) Pub. Date: Jul. 10, 2003

(54) Protein modeling tools

(76) Inventors: Jeffrey Skolnick, Creve Corner, MD (US); Andrzej Kolinski, Warsaw (PL)

Correspondence Address: GREGORY P. EINHORN, Fish & Richardson P.C., Suite 500, 4350 La Jolla Village Drive, San Diego, CA, 92122, US

(21) Appl. No.: 09/982,488

(22) Filed: Oct. 17, 2001

Related U.S. Application Data
(62) Division of application No. 09/493,022, filed on Jan. 27, 2000.
(60) Provisional application No. 60/117,570, filed on Jan. 27, 1999.
(60) Provisional application No. 60/118,844, filed on Feb. 5, 1999.

(51) Int. Cl. G06F 19/00; G01N 33/48; G01N 33/50

(52) U.S. Cl. 702/19

(57) ABSTRACT

The invention provides a new, efficient method for the assembly of protein tertiary structure from known, loosely encoded secondary structure constraints and sparse information about exact side chain contacts. The method is based on a new method for the reduced modeling of protein structure and dynamics, where the protein is described by representing side chain centers of mass rather than alpha-carbons. The model has implicit, built-in multi-body correlations that simulate short- and long-range packing preferences, hydrogen bonding cooperativity, and a mean force potential describing hydrophobic interactions. Due to the simplicity of the protein representation and definition of the model force field, the Monte Carlo algorithm is at least an order of magnitude faster than previously published Monte Carlo algorithms for three-dimensional structure assembly. In contrast to existing algorithms, the new method requires a smaller number of tertiary constraints for successful fold assembly; on average, one for every seven residues as compared to one for every four residues. The reliability and robustness of the invention make it useful for routine application in model building protocols based on various (and even very sparse) experimentally-derived structural constraints.

### RELATED APPLICATIONS

This application claims the benefit of priority under 35 U.S.C. § 119(e) of U.S. provisional patent application serial Nos. 60/117,570, filed Jan. 27, 1999, and 60/118,844, filed Feb. 5, 1999. Each of the aforementioned applications is explicitly incorporated by reference int heir entirety and for all purposes.

### GOVERNMENT INTERESTS

The instant invention was partially supported by a grant from the United States government under grant No GM-48835 awarded by the National Institutes of Health. As a result, the government may have certain rights in the invention.

### FIELD OF THE INVENTION

This invention concerns tools useful for modeling the three-dimensional structure of proteins. Specifically, the invention concerns algorithms, computer systems, and methods for determining, predicting, and/or refining three-dimensional structures of proteins.

### BACKGROUND OF THE INVENTION

The following description of the background of the invention is provided to aid in understanding the invention. It is not an admission that any of the information provided herein is prior art to the presently claimed invention, nor that any of the publications specifically or implicitly referenced are prior art to that invention.

A central tenet of modern biology is that heritable genetic information resides in a nucleic acid genome, and that the information embodied in such nucleic acids directs cell function. This occurs through the expression of various genes in the genome of an organism and regulation of the expression of such genes. The pattern of which subset of genes in an organism is expressed at a particular time in a particular cell defines the phenotype, and ultimately cell and tissue types. While the least genetically complex organisms, i.e., viruses, contain on the order of 10-50 genes and require components supplied by a cell of another organism in order to reproduce, the genomes of independent, living organisms (i.e., those having a genome that encodes for all the information required for the organism to survive and reproduce) that are the least genetically complex have more than 400 genes (for example, Mycoplasma genitalium). More complex, multicellular organisms (e.g., mice or humans) contain genomes believed to be comprised of tens of thousands or more genes, each of which codes for one or more different expression products.

Some of these genes are transcribed, but not translated; thus, the final gene products of these genes are RNA molecules (for example, ribosomal RNAs, small nuclear RNAs, transfer RNAs, and ribozymes (i.e., RNA molecules having endoribonuclease catalytic activity). However, most RNAs are mRNAs, and these are translated into proteins. The particular sequence of the ribonucleotides incorporated into an RNA as it is synthesized is dictated by the gene found in the genomic DNA from which it was transcribed. In the translation of an mRNA, the particular nucleotide sequence determines the particular amino acid sequence of the protein translated therefrom, and it is a protein's amino acid sequence that ultimately determines its three-dimensional structure, talking into account the thermodynamics of the system in which the protein is assembled. Significantly, three-dimensional structure dictates the particular biological function(s) of any biomolecule, including proteins.

The elegant simplicity of the foregoing schema is obscured by the complexity and size of the genomes found in living systems. For example, the haploid human genome comprises about 3×10⁹ (three billion) nucleotides spread across 23 chromosomes. However, it is currently estimated that less than 5% of this encodes the approximately 80,000-100,000 different protein-coding genes believed to be encoded by the human genome. Because of its tremendous size, to date only a portion of the human genome has been sequenced and deposited in genome sequence databases, and the positions of many genes and their exact nucleotide sequences remain unknown. Moreover, the biological function(s) of the gene products encoded by many of the genes sequenced so far remain unknown. Similar situations exist with respect to the genomes of many other organisms.

Notwithstanding such complexities, numerous genome sequence efforts designed to determine the exact sequence of the nucleotides found in genomic DNA of various organisms are underway and significant progress has been made. For example, the Human Genome Project began with the specific goal of obtaining the complete sequence of the human genome and determining the biochemical function(s) of each gene. To date, the project has resulted in sequencing a substantial portion of the human genome (J. Roach, http://weber.u.washington.edu/˜roach/human_genome_progress2.html) (Gibbs, 1995), and is on track for its scheduled completion in the near future. At least twenty-one other genomes have already been sequenced, including, for example, M. genitalium (Fraser et al., 1995), M. jannaschii (Bult et al., 1996), H. influenzae (Fleischmann et al., 1995), E. coli (Blattner et al., 1997), and yeast (S. cerevisiae) (Mewes et al., 1997). Significant progress has also been made in sequencing the genomes of model organisms, such as mouse, C. elegans, and D. melanogaster. Several databases containing genomic information annotated with some functional information are maintained by different organizations, and are accessible via the internet, for example, http:/www.tigr.org/tdb; http://www.genetics.wisc.edu; http://genome-www.stanford.edu/˜ball; http://hiv-web.lanl.gov; http://www.ncbi.nlm.nih.gov; http://www.ebi.ac.uk; http://pasteur.fr/other/biology; and, http://www-genome.wi.mit.edu.

Such sequencing projects result in vast amounts of nucleotide sequence information, which is typically deposited in genome sequence databases. However, these raw data (much of it being known only at the cDNA level), being devoid of corresponding information about genes and protein structure or function, are in and of themselves of extremely limited use (Koonin, et al. (1998), Curr. Opin. Struct. Biol., vol. 8:355-363). Thus, the practical exploitation of the vast numbers of sequences in such genome sequence databases is crucially dependent on the ability to identify genes and, for example, the function(s) of gene-encoded proteins.

To maximize the utility of such nucleotide sequence information, it must be interpreted. Various tools have been developed to assist in this process. For example, algorithms have been developed to analyze what a particular nucleotide sequence encodes, e.g., a regulatory region, an open reading frame (ORF), particularly for protein sequences, or a non-translated RNA, based on homology with known sequences (which are presumed to have similar structures and related functions). See, e.g., “Frames” (Genetics Computer Group, Madison, Wis.; www.gcg.com), which is used for identifying ORFs. For sequences predicted or determined to be ORFs, it is possible to determine the amino acid sequence of the protein encoded thereby using simple analytical tools well known in the art. For example, see “Translate” (Genetics Computer Group, Madison, Wis.; www.gcg.com). However, to date determination of the primary structure of a protein in and of itself provides little, if any, functional information about the protein or its corresponding gene. Thus, the ability to predict the three-dimensional structure of a protein from its amino acid sequence is of great theoretical^{1,2} and practical importance.³

In practice, structure prediction can be attempted on various levels, ranging from purely de novo, or “ab initio,” approaches to those that incorporate constraints derived from experimental data. The latter aspect of protein structure modeling has recently attracted significant attention⁴⁻⁶ due to its possible application to model building based on structural constraints provided by nuclear magnetic resonance (NMR),⁷ x-ray crystallography, or other experimental methods.

Perhaps the most useful method developed to date for predicting three-dimensional protein structures is the MONSSTER (Modeling of New Structures from Secondary and Tertiary Restraints) algorithm.⁸ MONSSTER provides a well-defined protocol for identifying moderate-resolution native-like three-dimensional structures from known secondary structure and a small number of tertiary constraints based on alpha-carbon (“Cα”) positions the amino acid residues of the protein.

That having been said, when a large number of distance constraints between atoms comprising amino acid residues of a protein are obtained from NMR or other experimental methods, and possibly from homology-based theoretical models of protein structure, more standard algorithms⁹⁻¹² are the tools of choice. These algorithms are based on purely geometrical considerations, followed by restrained molecular dynamics refinement of the model structures.¹³ However, in many real life situations, the number of available geometric constraints (e.g., interatomic distances, bond angles, etc.) is relatively small and limited, particularly in the early stages of structure determination based on experimental methods such as NMR. When the available geometric constraints are too sparse to define even a moderate resolution structure (i.e., a cRMSD of about 4-6 Å), it is necessary to use modeling methods that employ a reasonable force field capable of providing an overall protein-like bias. In such a case, even a small number of distance constraints could be sufficient to guide folding to the correct structure. Due to the necessity of sampling a substantial part of the protein conformational space, any such an algorithm should be computationally efficient. Moreover, the force field of the model should be able to correct for. The MONSSTER method is relatively efficient from a computational standpoint and can compensate for some errors in the provided set of Cα distance constraints. Significantly, however, even though MONSSTER is relatively efficient, the computational demands of that method have limited its application to proteins containing no more than 150 amino acid residues.

In the past several years, there also have been a number of other studies that have utilized experimentally derived secondary structure and a limited number of known, experimentally derived tertiary constraints to predict the global fold of a globular protein. In particular, Smith-Brown et al.⁴ reported the modeling of a protein as a chain of glycine residues. Tertiary constraints were reported to have been encoded via a biharmonic potential, with folding forced to proceed sequentially via successive implementation of those constraints. Using such methods, those authors reported that a substantial number of tertiary constraints were required to assemble a three-dimensional protein structure.

Another effort to predict the global fold of a protein from a limited number of distance constraints has been reported by Aszodi et al.⁵ Their approach was based on the use of distance geometry where a set of experimentally derived tertiary distance constraints was supplemented by a set of predicted distances between amino acid residues. The predicted distances were reported as being obtained from patterns of conserved hydrophobic amino acid residues that had been extracted from multiple sequence alignments with respect to the parent sequence. In general, they reported that when assembling structures below 5 Å cRMSD, on average, more than N/4 constraints are required, where “N” is the number of amino acid residues in the protein. Even then, the method reported by Aszodi et al. had difficulty selecting the correct fold from competing alternatives, although the approach was very rapid, with a calculation taking on the order of minutes on a typical contemporary workstation.

### SUMMARY OF THE INVETION

It is the object of this invention to provide new algorithms, methods, and computer systems that employ partial knowledge of known protein secondary structure and a small number of tertiary, or long range, constraints to determine the three-dimensional structure of a “target” protein. Here, “partial knowledge” means that there is no requirement for a detailed description of the local secondary structure in terms of φ and Ψ bond angles or their lattice equivalent. Instead, a three-letter code for secondary structure (H-helix, E-extended, and (-) everything else) is used as an input, wherein each amino acid residue of the protein is assigned an H, E, or -code. For a given protein, its corresponding H/E-code is translated by software into loosely defined preferred ranges of local intrachain distances. It is not necessary that all, or even a part, of the three-dimensional structure of the target protein be known, as the invention can be practiced using primary amino sequence information, whether derived from protein sequencing experiments or deduced from the coding region of a nucleic acid encoding the protein.

In particular, the invention relates to a new lattice protein model, termed “SICHO” (Side Chain Only), that focuses explicitly on the side chain center of mass positions of the amino acid residues of a target protein, and treats the protein backbone. The force field used in SICHO comprises short-range interactions that reflect secondary propensities and short-range packing biases, a geometrically implicit model of cooperative hydrogen bonds, and explicit burial, that is residues buried in the protein core and not exposed to water, pair interactions between side chains, and multi-body, involving three or more side chains tertiary interactions. The advantages afforded by the invention are due to more efficient protein representations and a new definition of the model force field that, when combined with a small number of long-range harmonic constraints (e.g. known side chain contacts), result in rapid collapse and assembly of a three-dimensional structure of the target protein. Additionally, because of the way the model and force field are implemented, SICHO's computational efficiency scales with a lower portion of the chain length, i.e., the number of amino acid residues comprising the target protein. Accordingly, the invention provides for the rapid, computationally efficient generation of one or more three-dimensional structures of one or more target proteins of known or deduced amino acid sequence.

Thus, a first aspect of the invention concerns methods for converting an alignment of a probe or “target” amino acid sequence with a template amino acid sequence into one or more three-dimensional reduced protein models comprising representations of side chains of amino acid residues comprising the target amino acid sequence. In some embodiments, the target amino acid sequence comprises a sequence of all of the amino acid residues of a protein. In other embodiments, the target amino acid sequence comprises a sequence of less than all of the amino acid residues of a protein, for example, a protein fragment or protein domain. A “probe amino acid sequence” is a sequence of amino acid residues whose three-dimensional structure or a “target amino acid sequence” is being determined byt hemethods of the invention, and can also be referred to as a “target” amino acid sequence, protein, protein fragment, or domain. In some embodiments of the invention, the target amino acid sequence will be deduced from a nucleotide sequence.

A “template” amino acid sequence refers to a sequence of amino acid residues against which the target amino acid sequence is comparatively aligned. Typically, the template amino acid sequence, in addition to having a known sequence of amino acid residues, will also comprise structural or conformation information. For example, such information can include secondary, supersecondary, tertiary, or quaternary structural information.

Target and template amino acid sequences can be aligned by any suitable method. Representative alignment algorithms are described below, and any suitable alignment algorithm can be employed in the practice of the invention. In preferred embodiments, the alignment is a threading alignment, prepared by a threading algorithm.

In various embodiments, the conversion of an alignment of a target amino acid sequence with a template amino acid sequence into one or more three-dimensional reduced protein models comprising representations of side chains of amino acid residues comprising the target amino acid sequence is performed using a computer. The alignment is input into the computer (for example, from a data storage device, another computer, a user interface, etc.), and a program, or computer control logic, instructs the computer (typically the processor, one or more which may be present depending on the computer used) to manipulate the alignment to produce a three-dimensional reduced protein model. Preferably, several different models are produced from any given alignment by varying one or more of the constraints imposed by the program. Each of the models can be output from the computer to an output device, e.g., a projection system (for example, a monitor) or to another device, such as a storage device. Preferably, the lowest energy model, or several low energy models (for example, 2-10 ), is(are) retained for a given target amino acid sequence. If desired, that model can then be used for various purposes, for example, to view the three-dimensional structure of the target amino acid sequence or by another computer program, e.g., a program that can identify protein functional sites. A reduced model according to the invention can also be used to build more refined, or detailed, structural models, including heavy atom models and all-atom models.

Another aspect of the invention concerns computer programs that can convert an alignment of a target amino acid sequence with a template amino acid sequence into one or more three-dimensional reduced protein models comprising representations of side chains of amino acid residues comprising the probe amino acid sequence. In certain embodiments, such programs utilize at least one secondary constraint and one tertiary constraint for each side chain center of mass present in the probe amino acid sequence. In other embodiments, only some of the amino acid residues represented in the probe amino acid sequence have at least one tertiary and/or at least one secondary constraint that is acted on by the computer program. Embodiments of secondary constraints include those indicating the presence of a helix, and extended conformation, or anything else. Embodiments of tertiary constraints include positions in continuous three-dimensional space, positions lattice-based three-dimensional space, ranges of such positions, distances, ranges of distances, bond angles, ranges of bond angles, etc.

Embodiments of the invention that concern computer-assisted methods for determining a three-dimensional structure of a target amino acid sequence using a computer include those wherein the computer comprises a processor configured to receive and output data in accordance with executable code, i.e., a program or computer control logic. Such methods include first inputting into the computer an alignment of a probe amino acid sequence with a template amino acid sequence. Then, by way of executable code, the processor is directed to produce from the alignment a three-dimensional reduced protein model comprised of representations of side chains of amino acid residues comprising the target protein. This representation can then be output to an output device or to a storage device.

In preferred embodiments, the executable code comprises instructions for converting representations of the side chains of amino acid residues of the target protein to interaction centers (which can be represented as “beads” or pseudoatoms) connected by virtual covalent bonds. Each interaction center typically comprises a pseudoatom representing a center of mass of the side chain of the represented amino acid to which the interaction center corresponds, and each interaction center, except for the interaction centers representing the amino and carboxy terminal amino acid residues of the protein, is connected to an immediately proximal interaction center and an immediately distal interaction center via a virtual covalent bond to produce an interaction center chain. The program then projects the interaction center chain onto an underlying cubic lattice to produce a projected chain of interaction centers. In many embodiments, interaction centers have identity constraints associated therewith. Secondary constraints and/or tertiary constraints are then applied to a subset of, or all of, the interaction centers of the interaction center chain so as to produce a data set representing a three-dimensional model structure of the target protein. This method can further comprise iterating the foregoing steps. In each iteration, a different set of secondary and/or tertiary constraints can be applied to the interaction centers to produce a series of data sets representing three-dimensional model structures of the target protein. An energy computation can then be made for each member of the series of data sets. The data set(s) having the lowest computed energy(ies) are then preferably retained. Preferably, 1, 2, 3, 4, 5, 6, 7, 8, 9, or 10 of the lowest energy data sets are retained or output to a data storage system to produce a stored data set. Alternatively, or in addition, one or more members of the data set can be output to an output device, such as a monitor on which the model can be visualized as a three-dimensional representation of the target protein. The member of the series of data sets having the lowest calculated energy can represent best, or highest quality, three-dimensional model structure of the target protein.

Definitions

The following terms have the following meanings when used herein and in the appended claims. Terms not specifically defined herein have their art recognized meaning.

As used herein, an “amino acid” is a molecule having the structure wherein a central carbon atom (the alpha (α)-carbon atom) is linked to a hydrogen atom, a carboxylic acid group (the carbon atom of which is referred to herein as a “carboxyl carbon atom”), an amino group (the nitrogen atom of which is referred to herein as an “amino nitrogen atom”), and a side chain group, R. When incorporated into a peptide, polypeptide, or protein, an amino acid loses one or more atoms of its amino and carboxylic groups in the dehydration reaction that links one amino acid to another. As a result, when incorporated into a protein, an amino acid is referred to as an “amino acid residue.” In the case of naturally occurring proteins, an amino acid residue's R group differentiates the 20 amino acids from which proteins are synthesized, although one or more amino acid residues in a protein may be derivatized or modified following incorporation into protein in biological systems (e.g., by glycosylation and/or by the formation of cystine through the oxidation of the thiol side chains of two non-adjacent cysteine amino acid residues, resulting in a disulfide covalent bond that frequently plays an important role in stabilizing the folded conformation of a protein, etc.). As those in the art will appreciate, non-naturally occurring amino acids can also be incorporated into proteins, particularly those produced by synthetic methods, including solid state and other automated synthesis methods. Examples of such amino acids include, without limitation, α-amino isobutyric acid, 4-amino butyric acid, L-amino butyric acid, 6-amino hexanoic acid, 2-amino isobutyric acid, 3-amino propionic acid, ornithine, norlensine, norvaline, hydroxproline, sarcosine, citralline, cysteic acid, t-butylglyine, t-butylalanine, phenylylycine, cyclohexylalanine, β-alanine, fluoro-amino acids, designer amino acids (e.g., β-methyl amino acids, α-methyl amino acids, Nα-methyl amino acids) and amino acid analogs in general. In addition, when an α-carbon atom has four different groups (as is the case with the 20 amino acids used by biological systems to synthesize proteins, except for glycine, which has two hydrogen atoms bonded to the a carbon atom), two different enantiomeric forms of each amino acid exist, designated D and L. In mammals, only L-amino acids are incorporated into naturally occurring polypeptides. Of course, the instant invention envisions proteins incorporating one or more D- and L-amino acids, as well as proteins comprised of just D- or L-amino acid residues.

As used herein, a “β-carbon atom” refers to the carbon atom (if present) in the R group of the side chain of an amino acid (or amino acid residue) that is covalently bonded to the α-carbon atom of that amino acid (or residue). For purposes of this invention, glycine is the only naturally occurring amino acid found in mammalian proteins that does not contain a β-carbon atom.

A “side chain center of mass” of an amino acid or amino acid residue refers to the calculated position in three-dimensional space of the center of mass of the sum total of the masses of all atoms comprising that side chain, although it may also include the alpha carbon and/or amino nitrogen of a particular amino acid or residue thereof. Herein, a side chain center of mass is preferably represented as a single pseudoatom.

Conventional amino acid residue abbreviations are used throughout this patent, and both the one and three letter codes are reproduced here for convenience: alanine=“A” or “Ala”; arginine=“R” or “Arg”; asparagine=“N” or “Asn”; aspartic acid=“D” or “Asp”; cysteine=“C” or “Cys”; glutamic acid=“E” or “Glu” glutamine=“Q” or “Gln”; glycine=“G” or “Gly”; histidine=“H” or “His”; isoleucine=“I” or “Ile”; leucine=“L” or “Leu”; lysine “K” or “Lys”; methionine=“M” or “Met”; phenylalanine=“F” of “Phe”; proline “P” or “Pro”; serine=“S” or “Ser”; threonine=“T” or “Thr”; tryptophan=“W” or “Trp”; tyrosine=“Y” or “Tyr”; and valine=“V” or “Val”. Amino acid sequences are written from carboxy- to amino-terminus, unless otherwise indicated. Conventional nucleic acid nomenclature is also used, wherein “A” means adenine, “C” means cytosine, “G” means guanine, “T” means thymine, and “U” means uracil. Nucleotide sequences are written from 5′ to 3′, unless otherwise indicated.

“Protein” refers to any polymer of two or more individual amino acids (whether or not naturally occurring) linked via a peptide bond, and occurs when the carboxyl carbon atom of the carboxylic acid group bonded to the α-carbon of one amino acid (or amino acid residue) becomes covalently bound to the amino nitrogen atom of amino group bonded to the α-carbon of an adjacent amino acid. These peptide bond linkages, and the atoms comprising them (i.e., α-carbon atoms, carboxyl carbon atoms (and their substituent oxygen atoms), and amino nitrogen atoms (and their substituent hydrogen atoms)) form the “polypeptide backbone” of the protein. In simplest terms, the polypeptide backbone shall be understood to refer the amino nitrogen atoms, α-carbon atoms, and carboxyl carbon atoms of the protein, although two or more of these atoms (with or without their substituent atoms) may also be represented as a pseudoatom.

The term “protein” is understood to include the terms “polypeptide” and “peptide” (which, at times, may be used interchangeably herein) within its meaning. In addition, proteins comprising multiple polypeptide subunits (e.g., DNA polymerase III, RNA polymerase II), as well as other non-proteinaceuos catalytic molecules (e.g., ribozymes) will also be understood to be included within the meaning of “protein” as used herein. Similarly, “protein fragments,” i.e., stretches of amino acid residues that comprise fewer than all of the amino acid residues of a protein, are also within the scope of the invention and may be referred to herein as “proteins.” Additionally, “protein domains” are also included within the term “protein.” A “protein domain” represents a portion of a protein comprised of its own semi-independent folded region having its own characteristic spherical geometry with hydrophobic core and polar exterior.

In biological systems (be they in vivo or in vitro, including cell-free, systems), the particular amino acid sequence of a given protein (i.e., the polypeptide's “primary structure,” when written from the amino-terminus to carboxy-terminus) is determined by the nucleotide sequence of the coding portion of a messenger RNA (“mRNA”) molecule, which is in turn specified by genetic information, typically plasmid or genomic DNA (which, for purposes of this invention, is understood to include organelle DNA, for example, mitochondrial DNA and chloroplast DNA, as well as forms of viral genomes integrated into the genomic DNA of a host cell). Of course, any type of nucleic acid which constitutes the genome of a particular organism (e.g., double-stranded DNA in the case of most animals and plants, single or double-stranded RNA in the case of some viruses, etc.) is understood to code for the gene product(s) of the particular organism. Messenger RNA is translated on a ribosome, which catalyzes the polymerization of a free amino acid, the particular identity of which is specified by the particular codon (with respect to mRNA, three adjacent A, G, C, or U ribonucleotides in the mRNA's coding region) of the mRNA then being translated, to a nascent polypeptide. Recombinant DNA techniques have enabled the large-scale synthesis of polypeptides (e.g., human insulin, human growth hormone, erythropoietin, granulocyte colony stimulating factor, etc.) having the same primary sequence as when produced naturally in living organisms. In addition, such technology has allowed the synthesis of analogs of these and other proteins, which analogs may contain one or more amino acid deletions, insertions, and/or substitutions as compared to the native proteins. Recombinant DNA technology also enables the synthesis of entirely novel proteins.

In non-biological systems (e.g., those employing solid state synthesis), the primary structure of a protein (which also includes disulfide (cystine) bond locations) can be determined by the user. As a result, polypeptides having a primary structure that duplicates that of a biologically produced protein can be achieved, as can analogs of such proteins. In addition, completely novel polypeptides can also be synthesized, as can proteins incorporating non-naturally occurring amino acids.

In a protein, the peptide bonds between adjacent amino acid residues are resonance hybrids of two different electron isomeric structures, wherein a bond between a carbonyl carbon (the carbon atom of the carboxylic acid group of one amino acid after its incorporation into a protein) and a nitrogen atom of the amino group of the α-carbon of the next amino acid places the carbonyl carbon approximately 1.33 Å away from the nitrogen atom of the next amino acid, a distance about midway between the distances that would be expected for a double bond (about 1.25 Å) and a single bond (about 1.45 Å). This partial double bond character prevents free rotation of the carbonyl carbon and amino nitrogen about the covalent bond therebetween under physiological conditions. As a result, the atoms bonded to the carbonyl carbon and amino nitrogen reside in the same plane, and provide discrete regions of structural rigidity, and hence conformational predictability, in proteins.

Beyond the peptide bond, each amino acid residue contributes two additional single covalent bonds to the polypeptide chain. While the peptide bond limits rotational freedom of the carbonyl carbon and the amino nitrogen of adjacent amino acids, the single bonds of each residue (between the α-carbon and carbonyl carbon (the phi (φ) bond) and between the α-carbon and amino nitrogen (the psi (ψ) bond) of each amino acid residue), have greater rotational freedom. For example, the rotational angles for φ and ψ bonds for certain common regular secondary structures are listed in the following table:

| | Approximate Bond | | | |
|---|---|---|---|---|
| | Angle | | Residues | Helix |
| Structure | φ | ψ | per turn | pitch (Å)^{a} |
| Right-handed α-helix | 57⁻ | 47⁻ | 3.6 | 5.4 |
| (3.6₁₃-helix) | | | | |
| 3₁₀-helix | 49⁺ | 26⁻ | 3.0 | 6.0 |
| Parallel β-strand | 119⁻ | 113⁺ | 2.0 | 6.4 |
| Antiparallel β-strand | 139⁻ | 135⁺ | 2.0 | 6.8 |

^{a}“Helix pitch” refers to the distance between repeating turns on a line drawn parallel to the helix axis. Bond angles associated with other secondary structures are known in the art, or can be determined experimentally using standard techniques.

Similarly, the single bond between a α-carbon and its attached R-group provides limited rotational freedom. Collectively, such structural flexibility enables a number of possible conformations to be assumed at a given region within a polypeptide. As discussed in greater detail below, the particular conformation actually assumed depends on thermodynamic considerations, with the lowest energy conformation being preferred.

In addition to primary structure, proteins also have secondary, tertiary, and, in multi-subunit proteins, quaternary structure. “Secondary structure” refers to local conformation of the polypeptide chain, with reference to the covalently linked atoms of the peptide bonds and α-carbon linkages that string the amino acid residues of the protein together. Side chain groups are not typically included in such descriptions. Representative examples of secondary structures include α helices, parallel and anti-parallel β structures, and structural motifs such as helix-turn-helix, β-α-62 , the leucine zipper, the zinc finger, the β-barrel, and the immunoglobulin fold. Movement of such domains relative to each other often relates to biological function and, in proteins having more than one function, different binding or effector sites can be located in different domains.

“Tertiary structure” concerns the overall three-dimensional structure of a protein, including the spatial relationships of amino acid residue side chains and the geometric relationship of different regions of the protein. “Quaternary structure” relates to the structure and non-covalent association of different polypeptide subunits in a multisubunit protein.

A “functional site” refers to any site in a protein that has a function. Representative examples include active sites (i.e., those sites in catalytic proteins where catalysis occurs), protein-protein interaction sites, sites for chemical modification (e.g., glycosylation and phosphorylation sites), and ligand binding sites. Ligand binding sites include, but are not limited to, metal binding sites, co-factor binding sites, antigen binding sites, substrate channels and tunnels, and substrate binding sites. In an enzyme, a ligand binding site that is a substrate binding site may also be an active site.

A “pseudoatom” refers to a position in three dimensional space (represented typically by an x, y, and z coordinate set) that represents the average (or weighted average) position of two or more atoms in a protein or amino acid. Representative examples of a pseudoatom include an amino acid side chain center of mass and the center of mass (or, alternatively, the average position) of an α-carbon atom and the carboxyl atom bonded thereto. Hypothetical covalent bonds between pseudoatoms, or between a pseudoatom and another atom, are referred to herein as “virtual covalent bonds.”

A “geometric constraint” or “tertiary constraint” refers to a spatial parameter with respect to an atom or group of atoms (e.g., an amino acid, the R-group of an amino acid, the center of mass of an R-group of an amino acid, a pseudoatom, etc.). Accordingly, such constraints can be represented by coordinates in three dimensions, for example, as having a certain position, or range of positions, along x, y, and z coordinates (i.e., a “coordinate set”). Alternatively, a geometric or tertiary constraint can be represented as a distance, or range of distances, between a particular atom (or pseudoatom, group of atoms, etc.) and another atom (or pseudoatom, group of atoms, etc.). Tertiary constraints can also be represented by various types of angles, including the angle of bonds (particularly covalent bonds, e.g., φ bonds and ψ bonds) between atoms in an amino acid residue, between atoms in different amino acid residues, and between atoms in an amino acid residue of a protein and another molecule, e.g., a ligand, with ranges for each angle being preferred.

A “conformational constraint” or “secondary constraint” refers to the presence of a particular protein conformation, for example, an α-helix, parallel and antiparallel β strands, leucine zipper, zinc finger, etc. in which an amino acid residue, or group of residues, is located. In addition, conformational or secondary constraints can include amino acid sequence information without additional structural information. As an example, “—C—X—X—C—” is a conformational constraint indicating that two cysteine residues must be separated by two other amino acid residues, the identities of each of which are irrelevant in the context of this particular constraint.

An “identity constraint” refers to a constraint that indicates the identity of a particular amino acid residue at a particular amino acid position in a protein. Typically, an amino acid position is determined by counting from the amino-terminal residue of the protein up to and including the residue in question. As those in the art will appreciate, comparison between related proteins may reveal that the identity of a particular amino acid residue at a given amino acid position in a protein is not entirely conserved, i.e., different amino acid residues may be present at a particular amino acid position in related proteins, or even in allelic or other variants of the same protein.

To “relax” a constraint refers to the inclusion of a user-defined variance therein. The degree of relaxation will depend on the particular constraint and its application.

As those in the art are aware, protein structures can be of different quality. Presently, the highest quality determination methods are experimental structure prediction methods based on x-ray crystallography and/or NMR spectroscopy. In x-ray crystallography, “high resolution” structures are those wherein atomic positions are determined at a resolution of about 2 Å or less, and enable the determination of the three-dimensional positioning of each atom (or at least each non-hydrogen atom) of a protein. “Medium resolution” structures are those wherein atomic positioning is determined at about the 2-4 Å level, while “low resolution” structures are those wherein the atomic positioning is determined in about the 4-8 Å range. Herein, protein structures that have been determined by x-ray crystallography or NMR may be referred to as “experimental structures,” as compared to those determined by computational methods, i.e., derived from the application of one or more computer algorithms to a primary amino acid sequence to predict protein structure.

As alluded to above, protein structures can also be determined entirely by computational methods, including, but not limited to, homology modeling, threading, and ab initio methods. Often, models produced by such computational methods are “reduced” models. A “reduced model” refers to a three-dimensional structural model of a protein wherein fewer than all heavy atoms (e.g., carbon, oxygen, nitrogen, and sulfur atoms) of the protein are represented. For example, a reduced model might consist of just the α-carbon atoms of the protein, with each amino acid connected to the subsequent amino acid by a virtual bond. In one embodiment, reduced models are those comprised only of side chain centers of mass. As will be appreciated by those in the art, more detailed model structures of a protein can be assembled from a reduced model. For example, a reduced model comprised only of amino acid residue side chain centers of mass implicitly specifies the location of the atoms comprising the side chain, as well the position of the peptide backbone. Accordingly, whatever greater level of atomic detail is required, if any, for the particular application can be added to a reduced model, and it is understood that once a protein structure based on a reduced model has been generated, all or a portion of it may be further refined to include additional predicted detail, up to including all atom positions.

Computational methods usually produce lower quality structures than experimental methods, and the models produced by computational methods are often called “inexact models.” While not necessary in order to practice the instant methods, the precision of these predicted models can be determined using a benchmark set of proteins whose structures are already known. For example, the predicted model can be compared to a corresponding experimentally determined structure. The difference between the predicted model and the experimentally determined structure is quantified via a measure called “root mean square deviation” (RMSD). A model having an RMSD of about 2.0 Å or less as compared to a corresponding experimentally determined structure is considered “high quality”. Frequently, predicted models have an RMSD of about 2.0 Å to about 6.0 Å when compared to one or more experimentally determined structures, and are called “inexact models”. As those in the art will appreciate, RMSDs can also be determined for one or more atomic positions when two or experimental structures have been generated for the same protein.

### BRIEF DESCRIPTION OF THE DRAWINGS

FIG. 1. Illustration of the protein chain representation. (A) For a short expanded fragment and (B) for a helical fragment. The solid circles correspond to explicitly simulated side chain centers of mass. The open circles indicate the expected positions of the α-carbons.

FIG. 2. Some examples of bonds connecting three successive side-chain united atoms. (A) The open circles in the upper panel correspond to a subset of possible positions of a third side chain given that the positions of the two preceding units (solid circles) are fixed and (B) illustration of excluded volume clusters. The solid dots correspond to the three lattice points along the axis orthogonal to the displayed slice. The open circles correspond to a single point in the plane.

FIG. 3. Examples of the conformational transitions employed in the Monte Carlo algorithm: (A) three examples of possible two-bond moves (the number of possibilities is much larger), (B) an example of a chain-end update, (C) an example of a three-bond move, and (D) a rigid body-like displacement of a larger portion of the model chain.

FIG. 4. Explanation of geometry used for the definition of the six terms describing the sequence-specific short-range interactions.

FIG. 5. Illustration of model hydrogen bond geometry. The hydrogen bonds are shown by open arrows.

FIG. 6. Geometry employed in the definition of the helical bias.

FIG. 7. Geometry employed in the definition of the extended, β-state bias.

FIG. 8. Fold of 3 fxn obtained using 20 tertiary restraints compared with the native structure, The picture has been prepared using MOLMOL⁴². The native secondary structure boundaries (helices and β-strands) have been superimposed on the predicted structure. A slight distortion of one helix (bottom right of the figure) and some distortions of the central β-sheet are noticeable.

FIG. 9. Representative structure of 4 fab obtained using 16 tertiary restraints compared with the native structure.

FIG. 10. Schematic illustration of a protein representation. The fragment of a detailed protein structure (main chain backbone and the side chains in thinner sticks) is shown in black. The gray sticks correspond to the virtual bonds of the model chains, connecting the centers of mass of groups of atoms consisting of side chains and alpha carbons.

FIG. 11. Lattice representation of the model chain and its excluded volume. The sticks correspond to the model chain virtual bonds. Excluded volume of each model amino acid is represented by 19 points on the underlying cubic lattice with the mesh size equal to 1.45 Å. The black dots correspond to three lattice points along the axis orthogonal to the picture plane (one in the plane, one below and one above the plane). The open circles correspond to single lattice points in the picture plane.

FIG. 12. A fragment of the model chain and a set of vectors w employed in the definition of the short-range polypeptide chain stiffness.

FIG. 13. Schematic illustration of the main chain's “hydrogen bonds”. Residue i is hydrogen bonded to residue j and k because the vectors hᵢ and −h_{I} connect with any of the points forming of the excluded volume clusters (the clusters are symbolically shown as large spheres) of these residues.

FIG. 14. Fragment of the model template chain (shown in the black sticks) and the template tube formed by the chain of spheres. The target chain (not shown in the drawing) is allowed to move in the tube with a penalty associated with all excursion from the tube.

FIG. 15. Flow chart illustrating the molecular modeling procedure described in the text.

FIG. 16. Stereo drawings of the two models of plastocyanin (in gray) superimposed onto crystallographic structure 2 pcy (in black). The upper panel shows the model obtained by MODELLER from the threading alignment, the lower panel shows the model obtained by the procedure described in this work. For the ease of illustration, only the alpha carbon traces are shown.

FIG. 17. Stereo drawings of the two models of the cytochrome 256 b (in gray) superimposed onto crystallographic structure (in black). The upper panel shows the model obtained by MODELLER from the threading alignment, the lower panel shows the model obtained by the procedure described in this work. For the ease of illustration, only the alpha carbon traces are displayed.

FIG. 18. Stereo drawings of the two models of telokin (in gray) superimposed onto crystallographic structure 1 tlk (in black). The upper panel shows the model obtained by MODELLER from the threading alignment, the lower panel shows the model obtained by the procedure described in this work. For the ease of illustration, only the alpha carbon traces are displayed.

FIG. 19. Displacement of the model chain units during the Monte Carlo simulation as a function of the position along the chain for the aligned portion of the 256 b molecule. The very stable (most of the second helix and C-terminal hairpin) regions and very mobile regions (the first helix and the central loop region) are clearly separated. This is the pattern typical for successful modeling (relatively low final RMSD from the native structure).

FIG. 20. Displacement of the model chain units during the Monte Carlo simulation as a function of the position along the chain for the aligned portion of the 5 fdl molecule. In contrast to the case of 256 b (see FIG. 19) the displacements of the chain elements are essentially random. This kind of pattern suggests a rather poor quality final model.

FIG. 21. Accuracy of the final models, measured as the C_{α} RMSD from the native structure, as a function of displacement variation. The variation is defined as a ratio of the number of passages of the residue displacement plot (as given in FIGS. 19 and 20) through the line of average displacement to the total number of protein residues.

These and other aspects and embodiments of the invention will be apparent to those in the art upon consideration of the detailed description, examples, claims, and figures, below, and such other aspects and embodiments shall be deemed to be a part of the invention as if they were described herein.

### DETAILED DESCRIPTION

The present invention is based on the discovery that accurate, useful three-dimensional structural models of target proteins whose tertiary structure is not known can be built using knowledge of protein secondary structure and a small number of tertiary constraints. In particular, it has been discovered that, when each amino acid residue of a protein is known (or deduced), and is converted into a representation based on the position of the side chain centers of mass for some or all of the protein's amino acid residues, accurate three-dimensional structures of the protein can be rapidly and efficiently generated. Preferably, the amino acid residues are classified as being positioned in a helix (“H”), extended (“E”), or other secondary structure (“(-)”), and software can be used to translate the code into loosely defined preferred ranges of local intrachain distances. As a result of this invention, three-dimensional structures of target proteins can be rapidly produced from primary amino sequence information, whether derived from protein sequencing experiments or deduced from the coding region of a nucleic acid encoding the protein.

Given the tremendous efforts currently underway to sequence the complete genomes of a variety of organisms, including humans, and the vast quantities of nucleotide sequence information be generated, the instant invention will be particularly useful to produce high, medium, or low resolution three-dimensional models of the structures of the proteins encoded amongst this newly identified nucleotide sequence data. Moreover, after producing such structures, they can be used as substrates to determine protein, and hence, gene function. In one embodiment, the instant invention can be used in processes where raw nucleotide sequence information is converted into amino acid sequence information. The amino acid sequence information is then converted into a three-dimensional structure of the protein comprised of those amino acid residues. The target protein's three-dimensional structure can then be used to determine its function. One or more steps of this process can be automated. Indeed, these steps can be automated so as to allow protein function to be assessed directly from primary amino acid sequence data, or even nucleotide sequence data that has been parsed to identify protein coding regions.

Embodiments of the invention are described in the following detailed description, which is outlined as follows. First, a discussion of proteins is provided, followed by a description of various alignment technologies. Next, a detailed description of SICHO is provided, including a detailed description of the geometric properties of the model, its force field, and the conformational sampling protocol. The description of SICHO is followed by a description of how the three-dimensional models produced thereby can be used, as well as how to implement the invention via a computer system. Examples describing the practice of the invention are then provided. The first example describes the results on the folding of eight representative proteins having a number of common protein motifs, and a comparison of these results with those reported previously.⁴⁻⁶

### PROTEINS

Under physiological conditions, each protein assumes a “native conformation,” a unique secondary and tertiary (and quaternary conformation in the case of multi-subunit proteins) conformation dictated by the protein's primary structure. The folding of a protein typically is spontaneous and under the control of non-covalent forces, and results in the lowest free energy state kinetically available under the particular pH, temperature, and ionic strength conditions. Disulfide bonds are typically formed after folding occurs, and serve to stabilize the native conformation. However, it is known that proteins having unrelated biological function or sequence can have similar patterns of secondary structure in the tertiary structure of different domains.

General protein folding parameters play an important role in predicting protein folding, and are based on observations that a protein's native conformation is spontaneously assumed by non-covalent interactions, although interactions with other proteins, for example, chaperoning, may be required for the proper folding of some proteins. Non-covalent interactions are weak bonding forces having bond strengths that range from about 4 to about 29 kcal/mol, which exceed the average kinetic energy of molecules at 37° C. (about 0.6 kcal/mol). In contrast, covalent bonds have bond strengths of least about 50 kcal/mol. While individually weak, the large number of non-covalent interactions in a polypeptide having more than several amino acids add up to a large thermodynamic force favoring folding.

Protein folding parameters include, among others, those relating to relative hydrophobicity, i.e., preference for the hydrophobic environment of a non-polar solvent. See Textbook of Biochemistry with Clinical Correlations, 3^{rd} Ed., ed. Devlin, T. M., Wiley-Liss, p. 30 (1992)). Hydrophobic interactions are believed to occur not because of attractive forces between non-polar groups, but from interactions between such groups and the water in which they are, or otherwise would be, dissolved. The salvation shell (a highly ordered, and therefore thermodynamically disfavored, arrangement of water molecules around a non-polar group) around a single residue is reduced when another non-polar residue becomes positioned nearby during folding, releasing water in the solvation shell into the bulk solvent and thereby increasing the entropy of water solvent. It is estimated that approximately one-third of the ordered water molecules in an unfolded protein's solvation shell are lost into the bulk solvent upon formation of a secondary structure, and that about another one-third of original solvation water molecules are lost when a protein having a secondary structure folds into its tertiary structure.

Amino acid residues preferring hydrophobic environments tend to be “buried,” i.e., those found at least about 95% of the time within the interior of a folded protein, although positioning on the exterior surface of a globular protein can occur by placing the more polar components of the amino acid near the exterior surface. The clustering of two or more non-polar side chains on the exterior surface are generally associated with a biological function, e.g., a substrate or ligand binding site. Polar amino acids are typically found on the exterior surface of globular proteins, where water stabilizes the residue's polarity. Positioning of an amino acid having a charged side chain in a globular protein's interior typically correlates with a structural or functional role for that residue with respect to biological function of the protein.

Another important protein folding parameter concerns hydrogen bond formation. A hydrogen bond (having bonding energies between about 1 to about 7 kcal/mol) is formed through the sharing of a hydrogen atom between two electronegative atoms, to one of which the hydrogen is covalently bonded (the hydrogen bond “donor”). Hydrogen bond strength depends primarily on the distance between the hydrogen bond donor and acceptor atoms, with high bond energies occurring when the donor and acceptor atoms are from about 2.7 Å to about 3.1 Å apart. Also contributing to hydrogen bond strength is bond geometry. Bonds having high energies typically have the donor, hydrogen, and acceptors disposed in a colinear fashion. The dielectric constant of the medium surrounding the bond can also influence bond strength.

Electrostatic interactions (positive and negative) between charged amino acid residues also play a role in protein folding and substrate binding. The strength of these interactions varies directly with the charge on each ion and inversely with the solvent's dielectric constant and distance between the charges.

Other forces to consider in protein folding concern van der Waals forces, which involve both attractive and repulsive forces that depend on the distances between atoms. Attraction is believed to occur through induction of a complementary dipole in the electron density of adjacent atoms when electron orbitals approach at close distances. The repulsive component, also called steric hindrance, occurs at closer distances when neighboring atoms' electron orbitals begin to overlap. With regard to these forces, the most favorable interaction occurs at the van der Waals distance, which is the sum of the van der Waals radii for the two atoms. Van der Waals distances range from about 2.8 Å to about 4.1 Å. While individual van der Waals interactions usually have an energy less than 1 kcal/mol, the sum of these energies for even a protein of modest size is significant, and thus these interactions significantly impact protein folding and stability, and, ultimately, function.

Yet another interaction playing a role in protein folding and function concerns that which occurs when two or more aromatic rings approach each other such that the plane of the π electron orbitals of the aromatic rings overlap. Such interactions can have attractive, non-covalent forces of up to about 6 kcal/mol.

Other factors to consider in determining folding of proteins include the presence or absence of co-factors such as metals (e.g., Zn²⁺, Ca²⁺, etc.), as well as other consideration known in the art.

Thermodynamic and kinetic considerations control the protein folding process. Without being tied to a particular theory, it is believed that folding begins through short-range non-covalent interactions between several adjacent (as determined by primary structure) amino acid side chain groups and the polypeptide chain to which they are covalently linked. These interactions initiate folding of small regions of secondary structure, as certain R groups have a propensity to form α-helices, β structures, and sharp turns or bends in the protein backbone. Medium and long-range interactions between more distant regions of the protein then come into play as these distant regions become more proximate as the protein folds.

### ALIGNMENT TECHNIQUES

Many sequence alignment methods are known in the art, such as BLAST (Altschul et al., 1990), BLITZ (MPsrch) (Sturrock & Collins, 1993), and FASTA (Pearson & Lipman, 1988). Alignment methods such as these are typically employed to align amino acid sequences in order to determine the extent of amino acid sequence identity between an experimental, or “probe” or “target” amino acid sequence and one or more already stored sequences (the “template” amino acids sequence(s)).

Homology modeling can also be applied, particularly for amino acid sequences that are evolutionarily related, i.e., they are homologous, such that their residue sequences can be aligned with some confidence. In one example of this method, the sequence of a protein whose structure has not been experimentally determined can be aligned to the sequence of a protein whose structure is known using one of the standard sequence alignment algorithms (Altschul, et al. (1990), J. Mol. Biol., vol. 215:403-410; Needleman and Wunsch (1970), J. Mol. Biol., vol. 48:443-453; Pearson and Lipman (1988), Proc. Natl. Acad. Sci. USA, vol. 85:2444-2448). Homology modeling algorithms, for example, Homology (Molecular Simulations, Inc.), build the sequence of the protein whose structure is not known onto the structure of the known protein to produce a “homology model”.

An alternative approach to amino acid sequence alignment involves “threading” or “inverse folding” approaches. In such methods, one “threads” a probe amino acid sequence through different template structures and attempts to find the most compatible structure for a given sequence. In certain embodiments, sequence-to-structure alignments are performed by a “local-global” version of the Smith-Waterman dynamic programming algorithm (Waterman, 1995). In such embodiments, alignments are ranked by one or more, preferably three, different scoring methods. In a three method approach (Jaroszewski et al., 1997), the first scoring method can be based on a sequence-sequence type of scoring. In this sequence-based method, the Gonnet mutation matrix can be used to optimize gap penalties, as described by Vogt and Argos (Vogt et al., 1995). The second method can use a sequence-structure scoring method based on the pseudo-energy from the probe sequence “mounted” in the structural environment in the template structure. The pseudo-energy term reflects the statistical propensity of successive amino acid pairs (from the probe sequence) to be found in particular secondary structures within the template structure. The third scoring method can concern structure-structure comparisons, whereby information from the known template structure(s) is(are) compared to the predicted secondary structure of the probe sequence. A particularly preferred secondary structure prediction scheme uses a nearest neighbor algorithm.

After computing scores for the sequence-to-structure alignments, the statistical significance of the each score is preferably determined by fitting the distribution of scores to an extreme value distribution, and the raw score is compared to the chance of obtaining the same score when comparing two unrelated sequences (Jaroszewski et al., 1997).

Once the alignment of the probe sequence-to-template structure has been determined, it can be used in accordance with a side chain modeling algorithm according to the invention. When a threading algorithm is used in the practice some embodiments of this invention, the probe amino acid can be “threaded” through a large database of proteins whose structures have been experimentally elucidated by, for example, x-ray crystallography or NMR spectroscopy. U.S. Pat. No. 5,436,850, describes threading algorithms that can be used in the practice of this invention.

### SICHO

SICHO is a new lattice protein model that represents a significant advance in our ability to computationally derive three-dimensional protein structures. In particular, SICHO focuses explicitly on the side chain center of mass positions of the amino acid residues of a target protein. The force field used in SICHO comprises short-range interactions that reflect secondary structure propensities and short-range packing biases, a geometrically implicit model of cooperative hydrogen bonds, and explicit burial, pair, and multibody tertiary interactions. When this new model force filed is combined with a small number of long-range harmonic constraints (e.g., known side chain contacts), accurate three-dimensional reduced models of least medium resolution can be rapidly and efficiently generated for a given target protein.

Protein Representation

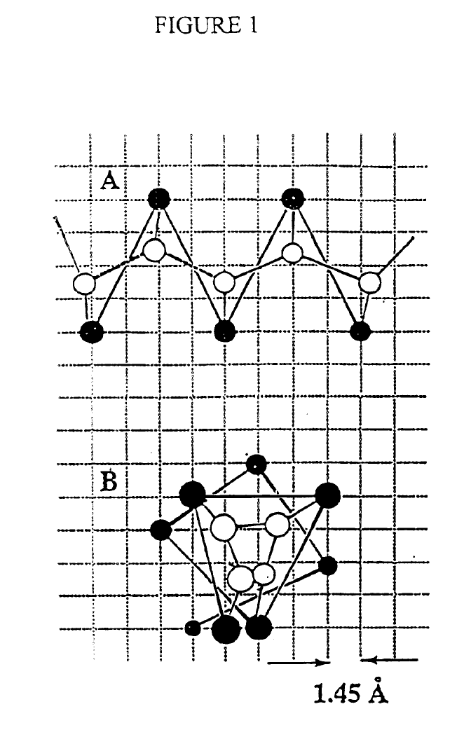

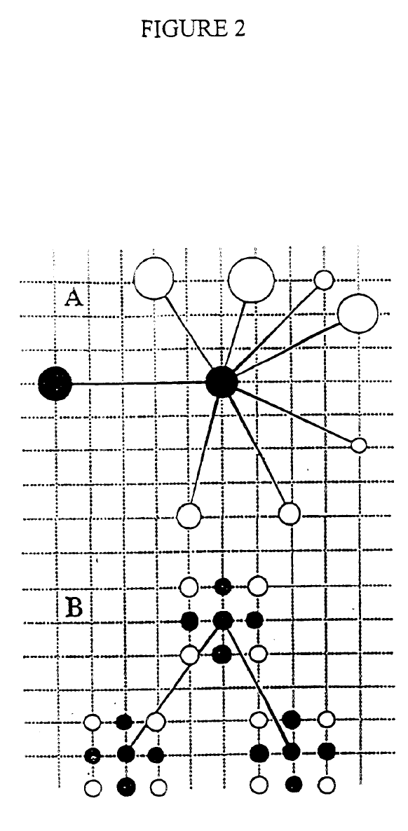

In SICHO, a target protein is modeled as a lattice chain connecting points restricted to an underlying simple cubic lattice whose mesh size equals 1.45 Å. By way of illustration, FIG. 1 depicts short fragments of a β-strand and an α-helix in this particular lattice representation. This figure also shows the corresponding Cα-traces, which are not explicitly modeled by SICHO, but can be back-filled after the three-dimensional model is generated, if desired, as other or even greater levels of detail can be. The distance between two consecutive side chain units is variable and is assumed to be in the range of 11½-30½ lattice units, or equivalently 4.8-7.9 Å. The length distribution roughly covers typical distances between two consecutive side chain centers of masse seen in real proteins.¹⁴ The resulting number of side chain vectors, {v}, is equal to 592. Similar limitations are superimposed on the distances between the i−th and i+2^{nd} side chain center of mass, i−th and i+3^{rd} side chain center of mass, etc., up to and including the i+8^{th} side chain center of mass. As a result, implicit limitations are superimposed onto the range of planar angles defined by the positions of three consecutive side chains. Some possible three-vector local conformations are shown in FIG. 2A.

As shown in FIG. 2B, the excluded volume cluster defined for each side chain consists of the central lattice point coinciding with the hypothetical center of mass of the side chain and the 16 surrounding points located at positions (=1,0,0) and (=1,=1,0), including all permutations of these vectors. With such a hard-core definition, the distance of closest approach of two residues is equal to three lattice units (4.35 Å). This corresponds to the equivalent hard core in observed in proteins for which a high resolution three-dimensional structure has been experimentally determined. There are also 30 possible lattice positions at which the closest approach, side chain-side chain contact, can occur. These are defined by six vectors of the (3,0,0) type and 24 vectors of the (2,2,1) type emanating from the side chain of interest. For larger residues, tryptophan, phenylalanine, tyrosine, histidine, and modified side chains of similar size (with similar criteria imposed for modified side chains based in their radius of gyration), a wider, finite magnitude, repulsive core is also included, and the number of “contact positions” is even larger. Consequently, effects of lattice anisotropy are essentially nonexistent.

Side chain overlaps and interactions are readily detected by inspection of the occupancy status of the appropriate collection of lattice points in the Monte Carlo working box. As a result, for a given amino acid residue, the computational cost for calculating the short- and long-range interactions does not depend on chain length.

Monte Carlo Model and Conformational Updating

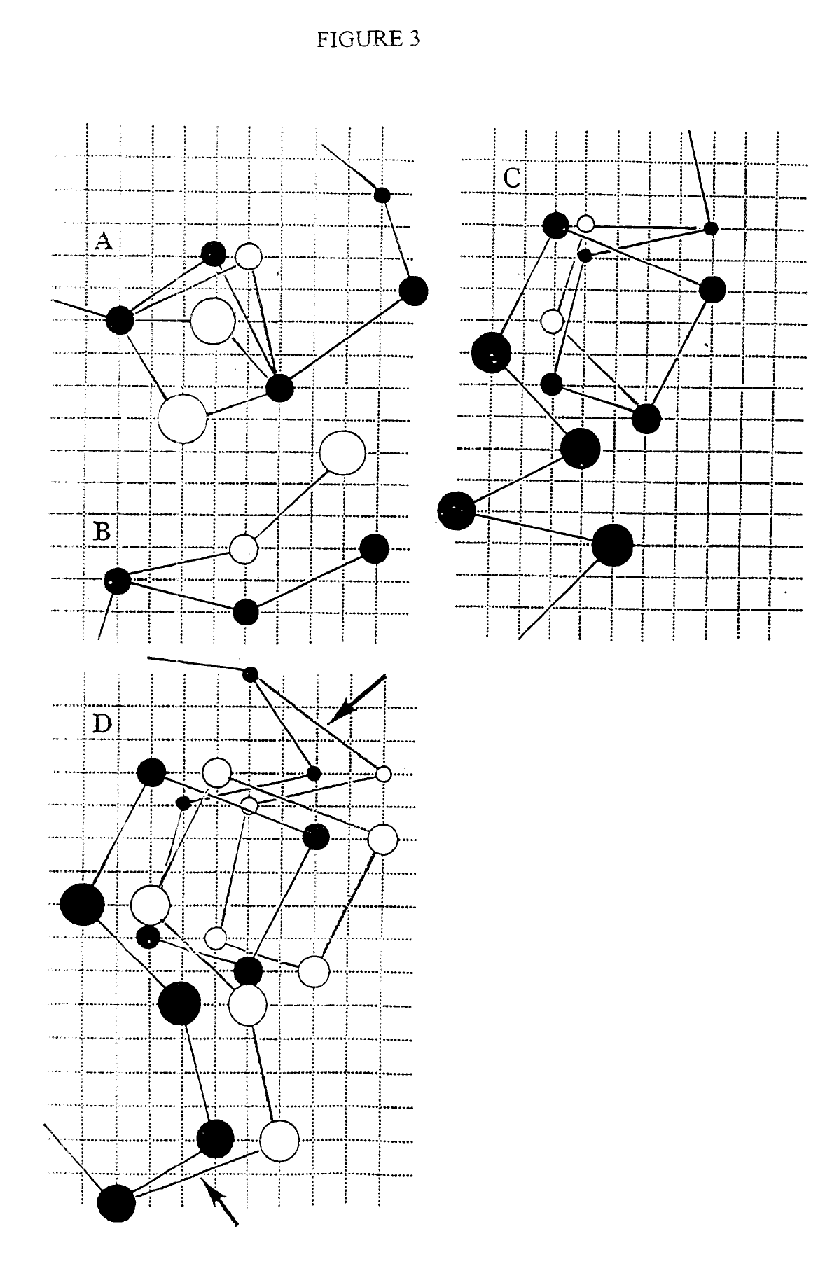

The Monte Carlo move set consists of single residue “kink” moves, chain-end moves, two-residue moves and small “rigid-body” displacements of a larger portion of the model chain. Examples of these moves are schematically illustrated in FIGS. 3A-D. A single “time-step” consists of N attempts at kink moves, 2 attempts at chain-end moves, N-1 attempts at two-bond moves and one attempt at a randomly selected, large fragment displacement. Here, “N” equals the number of amino acid residues in the protein. Before any energy computation, a test for excluded volume violations is performed, and trial conformations that would lead to steric collisions of chain units are rejected, as are conformations that would result in nonphysical distances between two consecutive side chain units.

Interaction Scheme

The interaction scheme employed in SICHO comprises short-range interactions, hydrogen bond interactions, and long-range interactions. All types of interactions have generic (i.e., sequence-independent), sequence-dependent, and target (i.e., resulting from superimposed short- and long-range constraints) components. Below, the generic and sequence-dependent terms are described first, followed by a description of those terms arising from the constraint contributions.

Sequence-Dependent Short-Range Interactions

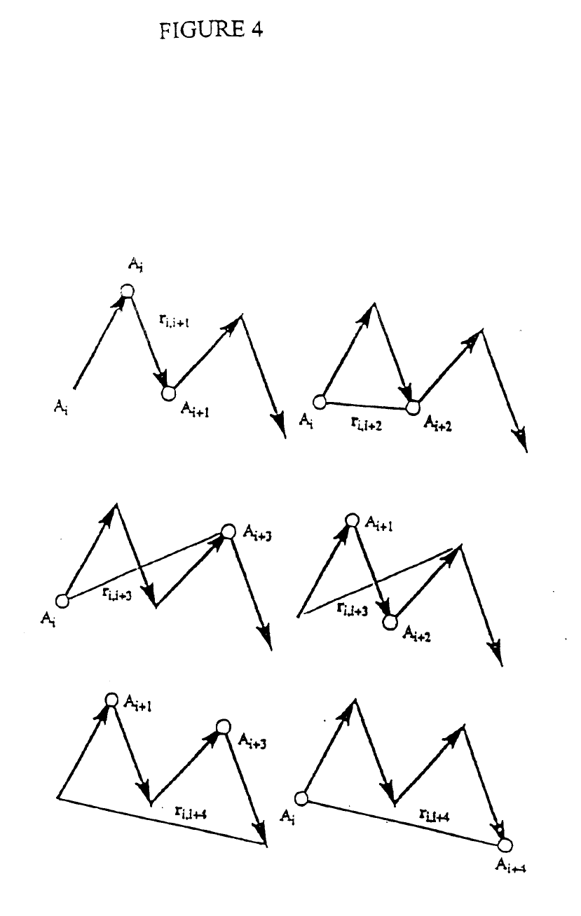

The potentials were derived from the geometric statistics of known protein structures. Pairwise-specific distances between nearest neighbors, up to the fourth neighbor, along the polypeptide chain are considered. These distances depend on amino acid composition and the local chain geometry. Six bins, covering the majority of distances, including the more distant pairs, i.e., the wings of the distance distribution (which are cut off at 4.8-7.9 Å) observed in proteins, have been used for all components of the short-range interactions. For a given pair of amino acid residues, the distribution of associated distances between side chain centers of mass is extracted from a statistical analysis of a structural database of non-homologous proteins (the Holm Sander PDB select database of 1501 proteins). When compared to an average distribution (ignoring sequence information), this leads to a statistical potential. The technique is similar to that employed elsewhere.¹⁵ As schematically illustrated in FIG. 4, the resulting potential could be expressed as follows:

$$\begin{matrix}{E}_{short} = \sum {E}_{12} ( {r}_{i , i + 1}^{2} , {A}_{i} , {A}_{i + 1} ) + \sum {E}_{13} ( {r}_{i , i + 2}^{2} , {A}_{i} , {A}_{i + 2} ) + \\ \sum {E}_{14} ( {r}_{i , i + 3}^{2 *} , {A}_{i + 1} , {A}_{i + 2} ) + \sum {E}_{14}^{′} ( {r}_{i , i + 3}^{2 *} , {A}_{i} , {A}_{i + 3} ) + \\ \sum {E}_{15} ( {r}_{i , i + 4}^{2} , {A}_{i + 2} , {A}_{i + 3} ) + \sum {E}_{15}^{′} ( {r}_{i , i + 4}^{2} , {A}_{i} , {A}_{i + 4} ) .\end{matrix} \qquad ( 1 )$$

The summation is performed along the chain; E_{1d} refers to energy associated with interactions between the residue of interest and its d-1^{st} neighbor down the chain. Aᵢ denotes the amino acid identity at. position i, and r_{i,i-k} is the distance between residues i and i+k. The terms for the three-bond fragments include the effects of local chain chirality via a “chiral”-distance-squared term.

r_{i-1,i+2}²*=r_{i-1,i-2}²sign((vᵢ₋₁\mathring{x}vᵢ)·vᵢ₊₁) (2)

All terms are amino acid pair-specific because the presently available structural database do not support meaningful statistics for higher order terms. Thus, there is a single energy term for one-bond and two-bond fragments, and two types of binary potentials for three-bond and four-bond fragments. These sequence dependent short-range interactions also provide information about short-range packing regularities, e.g., the propensities for a particular side chain arrangement on a helical surface. For simplicity, the relative scaling of all terms is preferably taken to be equal to one. This scaling generates a reasonable level and identity of secondary structure. While other scaling factors could be used, the quality of the results drops off, for example, less than native secondary structure or too much and poor backbone geometry are derived. Since there are a large number of numerical values for these short-range potentials (six components, each having 20×20×6 pair-wise values for 6-bin histograms), the data been reported⁴⁴ and are available via anonymous ftp¹⁷.

Generic Short-Range Conformational Biases

Next, terms that do not depend on amino acid sequence are introduced into the model force field. Thus, the energy contribution from these terms depends only on specific chain geometry (regardless of protein sequence) and its magnitude is controlled by a single adjustable energetical parameter, ε_{gen}. These terms' purpose is to enforce a protein-like distribution of short-range conformations.

The first set of these terms accounts for the characteristic stiffness of polypeptide chains, which builds on the observation that there is a characteristic orientation of protein chain that could be conveniently defined by a vector orthogonal to a triangle formed by three consecutive centers of mass of the side chains. The corresponding conformational bias could be defined as follows:

E_{stiff}=−0.25 ε_{gen}Σ(wᵢ·wᵢ₊₄) (3)

where wᵢ is a vector orthogonal to the plane formed by the two consecutive virtual covalent bonds vᵢ₋₁ and vᵢ, ε_{gen} is an arbitrarily chosen energetic parameter equal to 1 k_{B}T in all potentials described in this section, here scaled by a factor equal to −0.25. The length of the orthogonal vectors wᵢ is about 4 lattice units, and they are also used for detection of “hydrogen bonds.” The dot product in the above equation is near its maximum value for extended, β-like states and for helices. The high value of this product is significant in a majority of typical turns and loop-type local conformations. Thus, the potential provides a bias towards these relatively rigid elements of protein secondary structure.

The second generic term provides a bias towards regular arrangements of secondary structure. In a random lattice chain, the distribution of distances between the i−th and i+4^{th} bead would be unimodal and close to a Gaussian distribution. On the other hand, the corresponding distance distribution between residues in native proteins is bimodal. The shorter distance peak corresponds to helical and turn conformations, while the more diffuse, longer distance peak corresponds to extended conformations. A term that adjusts the model to this bimodal distribution could be expressed as follows, with all distances in lattice units.

E_{struct}=ΣEs(i) (4a)

with:

Es(i)=−2ε_{gen}

for r_{i,i+4}²<33 and (vᵢvᵢ₊₃)>0 (4b)

or

Es(i)=−2ε_{gen}

for 48<r_{i,i+4}²<145 and (vᵢ₊₁vᵢ₊₂)<0 (4c)

The first set of conditions (equation 4(b)) describes a loosely defined, helical conformation, while the second (equation 4(c)) describes an extended, β-type fragment. Thus, equation 4(b) states that the distance between the i−th and i+4^{th} side chain in a helix has to be small (here, below about 8 Å). The second condition states that the chain has to make a slight turn. A corresponding set of conditions is defined for β-type expanded states. In both cases, the cut-off distances and the angular restrictions are selected in a very permissive way based on the observed distributions for native proteins. The permissive definition of local conformational biases drives the model system towards a loosely defined protein-like chain geometry, yet it still allows substantial local mobility. As mentioned before, in preferred simulations, the value of ε_{gen} has been assumed to be equal to 1 k_{B}T.

“Hydrogen Bonds” and Generic Packing Biases

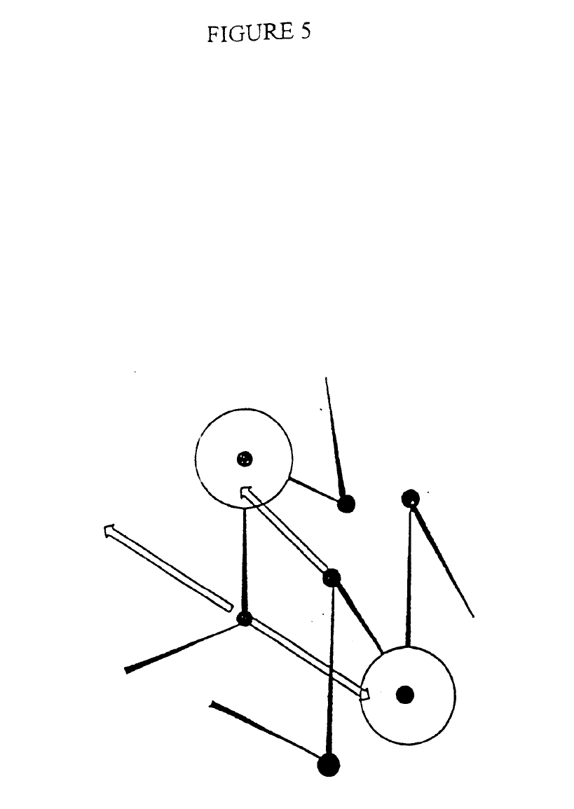

Model hydrogen bonds provide similar structure-regularizing biases with respect to tertiary interactions, as do the generic short-range interactions for secondary structural regularities. Residue i is considered to be hydrogen-bonded to residue j when the orthogonal vector wᵢ (originating from the bead i) touches any of the 17 points of the excluded volume cluster of residue j. In various embodiments of the model, two hydrogen bonds originate from a given residue. The geometry of hydrogen bonds is depicted in FIG. 5. Only residues that are “in contact” could be hydrogen-bonded. That is, there is the same long-range cut-off for side group pair interactions as for hydrogen bonding. The energy of the hydrogen bond network is defined as follows:

E_{H-bond}=−ε_{H-bond}Σ(δ⁺+δ⁻+δ^{+,-}) (5)

where δ⁺, δ⁻, δ^{+,-} are equal to 1 when the “right handed,” the “left handed,” and both hydrogen bonds originating from residue i are satisfied, respectively. Otherwise, the corresponding terms are equal to zero. The last term, δ^{+,-}, is a cooperative hydrogen bond energy gained only upon local saturation. The numerical value of this parameter was assumed to be equal to about 1.0-1.25 k_{B}T. Values of this parameter toward the lower end of the range tend to accelerate folding, while values toward the higher end tend to build structures of slightly better quality. In any event, these effects are small, and it is preferred to use a term having the same value (1.0) in all isothermal Monte Carlo runs used for energy comparisons.

Two other generic terms that enforce protein-like packing regularities also have been introduced. The first one is a “contact map propagator” that reflects the most common patterns seen in all side chain contact maps of globular proteins.¹⁸ It is defined in the following way:

$${E}_{map} = - {ϵ}_{gen} ( \sum \sum ( {δ}_{i , j} \cdot {δ}_{i + 1 , j + 1} \cdot {δ}_{i - 1 , j - 1} ) {δ}_{par} + \sum \sum ( {δ}_{i , j} \cdot {δ}_{i - 1 , j + 1} \cdot {δ}_{i + 1 , j - 1} ) {δ}_{apar} ) \qquad ( 6 )$$

where δᵢⱼ id equal to 1 (0) when residues i and j are (not) in contact. δₚₐᵣ is equal to 1 only when the corresponding chain fragments are oriented in a parallel fashion, i.e., (vᵢ₋₁+vᵢ)×(vⱼ₋₁+vⱼ). Similarly, δₐₚₐᵣ is equal to 1 when the chain fragments are anti-parallel. In the above equation and in equation 7, below, ε_{gen}=1 is the same parameter as the one used in the short-range generic terms.

A second packing regularizing term provides an additional cohesive energy between secondary structure elements by favoring the parallel packing of pairs of hydrophilic residues and the anti-parallel packing of pairs of hydrophobic residues. Consequently, since it exploits sequence information, this term is not purely generic; however, it is reduced to a two-letter (HP) code.

E_{packing}=−ε_{gen}ΣΣ(δ_{PP}·δₚₚ+δ_{HH}·δₐₚₚ) (7)

where δ_{PP} (δ_{HH}) is equal to 1 when both residues in contact are hydrophilic, P, (hydrophobic, H), according to the Kyte-Doolittle hydrophobicity scale.¹⁹ The value of δₚₚ is equal to 1 only when the packing of the side chain pair is parallel; i.e., (vᵢ₋₁−vᵢ)×(vⱼ₋₁−vⱼ)0. Similarly, δₐₚₚ is equal to 1 only when the packing of the side chain pair is parallel; i.e., (vᵢ₋₁−vᵢ)×(vⱼ₋₁−vⱼ)0.

Various structure regularizing terms described in this and the previous section reflect the various structural regularities seen in globular proteins. Each term accounts for a different correlation that could be easily detected by statistical analysis of the geometry of the side-chain-only representation of protein structures. Except for the last term (which depends on some sequence features), they are sequence independent: the underlying regularities are true for all types of structural motifs of globular proteins. During Monte Carlo simulations, these generic potentials provide a very strong bias against nonsensical, non-protein like conformations. Such conformations would otherwise be quite frequent due to the reduced character of the protein representation. In the presence of these generic contributions to the model force field, the requirements for sequence-specific potentials are lower; they have to select between various protein-like confirmations, which makes the selection easier (and computationally less expensive) than in the much broader conformational space of an unrestricted model chain.

Sequence-Specific Long-Range Interactions

These interactions are defined as follows:

Eₚₐᵢᵣ=ΣΣEᵢⱼ (8a)

where:

$${E}_{ij} = \begin{matrix}∝ & for {r}_{ij} < 3 \\ {E}^{rep} , & for 3 \leq {r}_{ij} < {R}_{i , j}^{rep} \\ {∈}_{i , j} , & for {R}_{i , j}^{rep} \leq {r}_{ij} < {R}_{i , j} \\ 0 , & for {R}_{i , j} < {r}_{ij}\end{matrix} \qquad \text{(8b)}$$

where εᵢⱼ are the pair-wise interaction parameters,^{6,26} and the interactions are counted for all pairs, except the first nearest neighbors along the chain. A strong soft-core repulsive energy of about 4 kT can be used in the simulations. This term provides a lightly larger excluded volume for larger amino acids than that defined by the hard core. The values of the cut-off distances Rᵢⱼ^{rep} and Rᵢⱼ are given in Table I, below. The values of Rᵢⱼ were adjusted to approximately mimic the contact distances employed in the derivation of binary interactions parameters.²⁰ Here, a “native” interaction scale as described by Skolnick, et al.²⁰

TABLE I
Compilation of Pairwise Cut-off Distances
in Angstroms

| | | | Rᵢⱼ | Rᵢⱼ |
|---|---|---|---|---|
| Aᵢ | Aⱼ | Rᵢⱼ^{rep} | (attractive)^{a} | (repulsive) |
| Small^{b} | Small | 4.35 | 7.03 | 6.32 |
| Small | Large | 4.57 | 7.03 | 6.32 |
| Large | Large | 4.83 | 7.50 | 7.03 |

^{a}Attractive pair of amino acids.
^{b}Small amino acids are: Gly, Ala, Ser, Cys, Val, Thr, Pro.

One-Body Burial Interactions

To facilitate a rapid collapse of the model chain, a centro-symnimetric, density regularizing term was used that is based on a statistical analysis of single domain proteins. This is the only term that uses the assumption that the target protein has a single domain. For some increase in computational cost, this term could be omitted. The radius of gyration of the protein is given by:

S=(N⁻¹Σ(r_{CM}−rᵢ)²)^{½} (9)

where r_{CM} is the position of the center of mass of the globule, and rᵢ is the position of the center of mass of the i−th side chain. The size of a single domain protein is strongly correlated with the number of residues, N, comprising the protein, in accordance with:

S=1.52 N^{0.38} in lattice units (10)

The exponent 0.38, obtained from the statistical analysis of single domain globular proteins,²¹ is very close to the value of ⅓ expected for a long, collapsed polymer chain.²² The corresponding potential has the following form:²³

E_{b}=ε_{b}Σ|m_{o.i}−mᵢ| (11)

where mₒᵢ is the target number of amino acids in a given spherical shell centered at the protein's center of mass. There are three equal thickness shells within a distance S, and they contain somewhat more than half of the protein residues. The entire protein is essentially contained in a sphere of radius equal to 5/3 S. The value of the parameter ε_{b} was equal to 0.25-1.0 k_{B}T, depending on protein size. Larger proteins tend to exhibit a larger absolute deviation from the above target distribution of mass, and consequently, a lower penalty for such deviations should be employed.

To further enhance rapid collapse, those residues that are within a radius of 2/3 S (a very conservative estimate of the hydrophobic core of a single domain globular protein) contribute ε_{KD}(i)/16 to the total energy, where ε_{KD}(i) is the Kyte-Doolittle hydrophobicity parameter of the i−th residue.^{19,24} The scaling factor 1/16 is preferred. This potential (and its scaling with respect to other interactions) has very little effect on the folded structure, but it improves folding kinetics.

Multibody Surface Exposure Term

Amino acid side groups have a different size and shape. Thus, when a given side chain is in contact with another amino acid, the fraction of its surface that is covered depends on the identity of the contacting partner. Appropriate parameters reflecting this observation (i.e., the surface coverage of particular types of side chains and associated statistical-type potential) could be derived from the statistics of known protein structures. In the present algorithm, each residue can have 30 surface contact points. A subset of these contact points becomes occupied upon contact with other side chains or main chain Cα atoms. The Cα atom positions are approximated from the positions of three consecutive side chain beads and have their own excluded volume and contribution to surface coverage. Due to “shadowing,” i.e., one residue being covered by another, some contact points could be multiply occupied by different residues (usually 1 or 2, or sometimes 3, but very rarely 4 or more). The fraction of occupied surface points defines the fraction of buried area of a given side chain. The total energy of a model protein is computed as:

E_{surface}=ε_{S}ΣE_{b}(Aᵢ, aᵢ) (12)

where aᵢ is the covered fraction of sites of amino acid side chain Aᵢ and E_{b} (Aᵢ, aᵢ) is the statistical potential for amino acids Ai that are covered by aᵢ contact points, i.e., its coverage fraction is a/30, when the number of contact points is 30. The reference state for this statistical potential is “an average” amino acid with average (over structural database) coverage. One scaling factor ε_{S} for this term has been determined to be 0.25, although other scaling can be used.

The above approach to the hydrophobic interactions allows suppression of previously employed centro-symmetric one-body potentials⁶ and thereby opens up the present approach to multi-domain and multi-meric proteins. In this example, both models of mean field hydrophobic interactions were used in parallel.

The force field designed for this model is entirely of a “knowledge-based” origin. Some terms, such as the generic short- and long-range potentials, provide a bias toward protein-like short- and long-range correlations in the model chain. These potentials generalize regularities seen in native structures of all globular proteins. The sequence-specific terms were derived as statistical potentials with a rather careful selection of the reference state.^{20,25,26} When several statistical potentials are combined in a relatively complex reduced model, an a priori derivation of the relative scaling factors becomes difficult. Some double counting of particular physical interactions may occur. Thus, these scaling factors have to be adjusted to reproduce a reasonable balance between the short- and long-range interactions. A proper balance should lead to a low secondary structure content in the denatured state and a well-packed and ordered collapsed state. The collapse transition should be as abrupt as possible, mimicking an all-or-none folding transition. This has been achieved in the present model with the given scaling of particular interactions. Folding experiments for several proteins of various structural classes were performed with no short- or long-range constraints. The force field described above fails to produce a unique folded state, except for very simple folding motifs. For more complex motifs, the folded states always had a secondary structure very close to the native, with good packing of the hydrophobic core; however, the arrangement of the secondary structure elements (connection of helices, order of β-strands in sheets, etc.) almost always had topological errors. As designed, the model with its force field is very efficient at generating protein-like compact conformations. The model is not sensitive to the particular scaling of the various interactions within a broad range around the set used in this work. For example, removal of all generic terms also led to collapsed structures (although at lower temperatures) with good overall fidelity of the secondary structure, but the geometrical accuracy of the secondary structure and packing pattern was more irregular. A detailed discussion of the interplay between the generic and sequence-specific short-range potentials is reported elsewhere.²⁷ When the proposed force field is supplemented by one or more structural constraints, a proper fold should be easily selected.

Since a Cα-based MONSTTER model has been reported as being successful in reproducing quite complex aspects of protein dynamics and thermodynamics^{6,15,16,23,28-36} without being bound to any particular theory, it is believed that the present force field approximately reproduces the main features of globular proteins. However, it does so in a different geometrical context, namely using pseudoatoms representing side chain centers of mass. Moreover, the instant invention is based on a less complex representation and simpler definition of the force field, and is more computationally more efficient than Cα-based models such as MONSSTER. As a result, three-dimensional structures for larger proteins can be simulated.

Physical Basis of the Model Interaction Scheme

The instant invention allows realistic three-dimensional protein structures (as seen on the level of an entire fold) to be produced from an extremely simplified representation of the protein conformational space. Here, only the side chains (in one embodiment represented by their respective centers of mass) are explicitly modeled. The use of a single interaction unit per residue is computationally very efficient. Moreover, side chains were used, as opposed to, for example, alpha-carbons, because the specific interactions between, or functions of, proteins involve side chains, while main chain (i.e., peptide backbone) interactions are much less dependent on amino acid sequence. Due to this very simple representation and requested specificity, several features have to be built into the model force field. First, the assumed protein representation, with a single center of interaction per amino acid residue side chain, allows too much conformational freedom. This is because there is no explicit backbone connectivity in the model chains. However, in real proteins, the backbone connectivity and conformational stiffness control, to some extent, the distances between the centers of mass of the side groups near each other along the polypeptide chain. The backbone effect is moderated by the side chains' internal degrees of freedom. It is reasonable to assume that for a short polypeptide fragment, the local geometry of the side chain centers of mass is mostly dictated by short-range interactions with a somewhat lesser effect from long-range (tertiary) interactions. The correct, protein-like distance geometry of the side chain centers of mass implies a correct, protein-like geometry of the main chain. This provides a conceptual background for the sequence-specific short-range potential of mean force (discussed previously and defined in equation 1, above). This potential drives the system towards a local geometry (characterized by distances between side chains) that is characteristic of locally similar sequences.

At first glance, it may appear that such defined sequence-specific secondary propensities are sufficient for modeling protein like local geometry. This is not the case for several reasons. First, the discussed statistical potentials are not very accurate due to the limited size of the database of known protein structures. However, more importantly, the assumed simplified representation of the polypeptide chains exhibits excessive flexibility. With respect to the assumed model of excluded volume, a substantial fraction of the model chain conformations that are otherwise allowed are conformations that cannot possibly occur in any protein or even in other polymers. It is not a good strategy to make the sequence-specific interactions so strong that the non-physical geometries would be practically prohibited. This would lead to dynamic frustration of the model system due to very frequent trapping in the local conformational energy minima; thus, providing a generic bias towards protein-like geometry is computationally more efficient. Then, much less is required from the sequence-specific part of the potential (selection within the protein-like part of conformational space instead of selection within a much larger conformational space of a freely joined polymer chain). Moreover, a properly defined generic potential can “interpolate” protein-like conformations for those fragments of a given polypeptide chain where the information content of the sequence-specific potential is low, (due to lack of examples in the database or balanced contradictory examples). As discussed above (see equations 3 and 4), sequence-independent potentials exactly play such a role. The first such term provides a bias towards the protein-like stiffness of the model chain by an energetic preference for either expanded zigzag or helical conformations. The second term provides a bias towards a bimodal distribution of the distances between the i−th and i+4^{th} side chain units. The definition of these potentials mimics some of the most general structural regularities seen in all folded proteins. They also provide a bias against nonphysical local conformations in the unfolded state.

Similar to the short-range interactions, there are sequence-specific and generic terms of the model tertiary interaction scheme. The pairwise contact potentials and the model of hydrophobic burial potentials of mean force derived from the statistics of the structural database do not require additional discussion. The procedures of derivation and implementation of such potentials are rather standard and commonly used in all reduced models of proteins.^{15-17,21,25,26} Such statistical potentials encode some interaction preferences in real proteins. In the majority of cases, they are accurate enough to select a proper fold for a given sequence from a collection of other folds of natural proteins. However, here, the requirements are more stringent. The proper fold must be selected from a much larger number of conformations, most of them never observed in real proteins (but possible in the model due to the reduced representation). Thus, it is important to construct a generic potential that provides a bias toward protein-like tertiary interaction patterns. Such patterns could be postulated as a generalization of structural regularities seen in known protein structures. An important feature of all protein structures is the very regular network of main chain hydrogen bonds. Our model lacks an explicit protein backbone. Nevertheless, an analysis of protein structures shows that the presence of hydrogen bonds between residues translates with high reproducibility into a pattern of contacting side chains. Indeed, the string of hydrogen bonds along a helix implies the existence of continuous or almost continuous strings of side group contacts along the helix surface. Similarly, a string of hydrogen bonds in a β-hairpin implies two strings of side group contacts, one on each side of the hairpin. Thus, a bias towards such a string (see equation 5, above, and the associated discussion of the potential) could be used as an ersatz copy of the hydrogen bond interactions. Furthermore, such strings of contacts lead to a characteristic pattern of side chain contacts. The generic potential given in equation 6, above, provides a bias towards the most general feature of such patterns.^{16,18}

Angular packing preferences for various types (hydrophobic or hydrophilic) of residues also could be used as a bias toward protein-like side chain packing patterns (see equation 7, above, and the associated description of this term).

Such a defined model of the force field can be tested and the relative weights of the sequence-specific versus generic terms adjusted by a trial and error method. Here, a long series of isothermal simulations of various proteins was performed. While the native-like structures were sometimes obtained only for very simple, small proteins, the accuracy (measured as cRMSD from native) of the emerging elements of secondary and super-secondary structure elements (helices, helical hairpins, β-hairpins or α-β-α motifs) could be used as a convenient criterion.

This force field alone, however, is not accurate enough for reproducible folding simulations of the majority of (even small) proteins. At the same time, it discriminates against a vast majority of nonsensical conformations, and the native-like structures always belong to a relatively small number of low energy conformations. Thus, when some long-range constraints of experimental origin are superimposed on top of this force field, the native-like conformation can easily be obtained in Monte Carlo simulations, as described below.

Implementation of the Constraints

Encoding Short-Range Conformational Propensities

In testing structure assembly using the methods described herein, knowledge of secondary structure³⁷ is used in the form of the following three-letter code: E-extended; H-helix; and (-) everything else. This three-letter code is then translated onto a set of biases towards a corresponding range of local intrachain distances and angular correlations. Only E and H states have some conformational biases, and their definitions are geometrically very permissive. The set of secondary structural constraints are as follows:

1. An H-state cannot be hydrogen bonded to an E-state. When detected, such bonds are ignored and do not contribute to the conformational energy.

2. A residue in a continuous stretch of H-states can hydrogen bond only to residues i−3 and i+3. Note that hydrogen bonds associated with Cα's or side chains represent the canonical helix pattern.

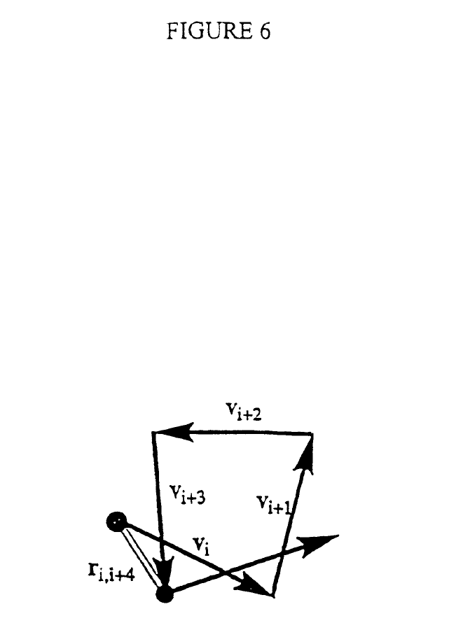

3. The system gains an additional energy equal to −ε_{gen} (over the previously defined generic contributions, and ε_{gen} is of the same exact value as that used in the definition of various generic germs of the model force field in all the following cases (a-c): As shown in FIG. 6 for helical type states when:

for r_{i,i+4}²<33 13(a)

a) residues i+1 and i+2 are assigned as helical if (vᵢ·vᵢ₊₂)<0

b) residues i+2 and i+3 are assigned as helical if (vᵢ₊₁·vᵢ₊₃)<0

c) residues i+1, i+2 nd i+3 are assigned as helical if (vᵢ·vᵢ₊₃)<0.

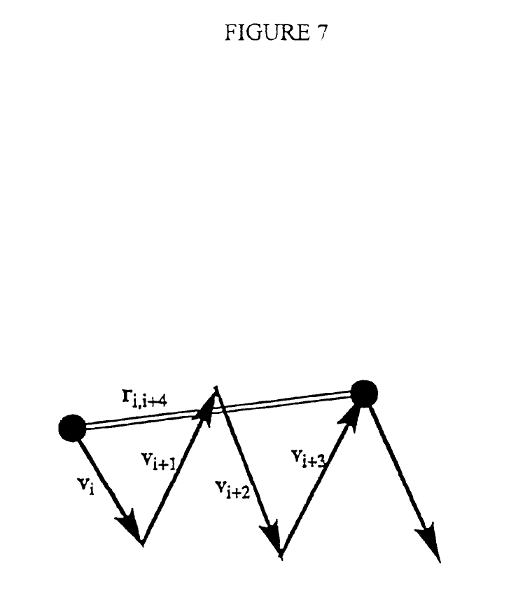

As shown in FIG. 7, for expanded states when

for 48<r_{i,i-4}²<145 13(b)

a) residues i+1 and i+2 assigned as extended if (vᵢ·vᵢ₊₂)<8

b) residues i+2 and i+3 assigned as extended if (vᵢ₊₁·vᵢ₊₃)<8

c) residues i+1, i+2 and i+3 assigned as extended if (vᵢ·vᵢ₊₃)<0.

The set of conditions given in equations 13a and 13b, above, describe various geometrical boundaries for the local conformation of the model chain that are characteristic for helical and expanded states, respectively. In each case, they were split into three sets of conditions to make the energy landscape as smooth as possible (otherwise, a single condition could be applied). In the present realization, the model system gains some energetic stabilization when even a nucleus of a helix or extended state forms. On the other hand, the conditions are rather permissive, allowing substantial fluctuations of the secondary structure without an energetical penalty. This is the reason for certain cut-offs for intrachain distances and dot products of the relevant side-chain vectors. Of course, these cut-offs are consistent with the vast majority of helical or β-type geometries seen in globular proteins. Of course, these terms may be modified or refined as additional three-dimensional proteins structures are solved to high resolution

Long-Range Constraints

Long-range constraints are implemented in the form of a distorted harmonic potential. Additionally, the contact energy for such side chain pairs is modified as well below:

$${E}_{ij , restrained} = \begin{matrix}∝ & for {r}_{ij} < 3 \\ {E}^{rep} , & for 3 \leq {r}_{ij} < {R}_{i , j}^{rep} \\ {∈}_{i , j} - 0.5 , & for {R}_{i , j}^{rep} \leq {r}_{ij} < {R}_{i , j} \\ {∈}_{res} ( {R}_{i , j}^{2} - {R}_{i , j}^{2 rep} & for {R}_{i , j} < {r}_{ij} < 10 \\ {∈}_{res} ( 100 - {r}_{ij}^{2} - 100 ) / 3 & for 10 < {r}_{ij}\end{matrix} \qquad ( 14 )$$

The value of parameter εᵣₑₛ in structure assembly runs was set equal to 1/8, while during the low temperature refinement run, it was set equal to ¼. The meaning of other parameters is the same as in equation 8, above. In the first three ranges, the above function is consistent with the definition of pairwise interactions defined in the previous section. For restrained residues, the pairwise potential has been enhanced (line 3 of equation 14). The two remaining lines define a pseudoharmonic long-distance potential. For longer distances (line 5 of equation 14), it is slightly suppressed because a weaker function facilitates a somewhat faster assembly of model protein chains.

Folding Procedure

The sampling procedure employed for protein assembly is based on Monte Carlo simulated thermal annealing. The stages are described below:

1. In the first step, a random expanded chain conformation is subjected to Monte Carlo simulated thermal annealing³⁸ over a broad range of temperature from T=6 (T=4 for smaller proteins) to T=1. After annealing, the number of satisfied long-range constraints in each folded protein is inspected. Those folds with more than about 1.7 of their constraints significantly violated are rejected without further inspection, e.g., when the corresponding side-chain:side chain distance is larger than 7 lattice units for proteins smaller than about 100 residues and 8 lattice units for proteins larger than about 100 residues. These alternative, exemplary parameters have been selected by studying a similar problem,⁶ and by preliminary testing of the present model. Allowing a significantly larger number of violated constraints may lead to topologically wrong folds, while requesting all constraints to be satisfied would decrease the efficiency of the method, as some good folds with small local distortions would be rejected. The success ratio at this stage depends on the protein and the number of long-range constraints. For example, in 1 gb1, protein G, with eight constraints, more than 75% of short assembly runs (5-15 minutes of CPU time on a HP C-110 workstation) are successful. In the case of 1 pcy, plastocyanin, with 15 constraints, the corresponding success rate is about 30% for 4-hour-long simulations on an HP C-110 workstation. Of course, a slower annealing protocol increases the fraction of assembled structures that satisfy the constraints. However, it appears that use of a larger number of shorter simulations is a more effective sampling protocol because a greater number of structures are collected for each protein.

2. All structures obtained via the rapid annealing procedure are preferably subjected to a refinement process. For refinement, each structure is duplicated and subjected to two independent Monte Carlo annealing runs over the temperature range T=2−1. The lowest conformational energy structure (from the last snapshot of the corresponding trajectories) is accepted for further analysis.

3. For each protein, both the lowest energy conformation and the lowest energy alternative conformation are then subjected to isothermal runs to establish whether the proper fold can be automatically selected based on the choice of the lowest average energy structure.

4. The Cα coordinates of the final, lowest energy structures are then built into the model. This “back filling” procedure is based on Monte Carlo annealing of a phantom lattice model chain that has two united atoms per residue: one centered on the Cα and the other at the side chain center of mass. This Cα plus side chain center of mass, CAPLUS, model (see, e.g., ^{6,16,29-31,39}) only employs statistical potentials describing short-range interactions and side chain rotamer preferences. The positions of the side chains in the CAPLUS model are driven by a harmonic potential to the predicted side chain positions from the side chain only model.

As those in the art will appreciate, other models of differing levels of detail, up to an including all heavy atom, and even all atom, representations, can also be assembled from these low energy structures. The level of detail and resolution chosen for these structures will typically depend on the particular application for which the model is intended. For example, rational drug design typically requires models having significant levels of atomic detail, particularly when protein:ligand interactions are being assessed.

APPLICATIONS AND AUTOMATED IMPLEMENTATION

Protein Function Determination

As described above, it is now possible to rapidly generate accurate, reduced protein models directly from nucleotide or deduced amino acid sequence data. These models, which are based on the side chain center of mass of the amino acid residues comprising a particular protein, can then be manipulated to produce other models, such as those depicting alpha-carbon atom representations, all heavy atom representations, and even all atom representations.

In alternative embodiments, such representations can be used to determine protein function using one or more three-dimensional templates correlated with particular biological functions, and they can also be used to identify functionally important regions in a protein. See, e.g., Kasuya, A. and Thornton, J. M., J. Mol. Biol., vol. 286: 1673-1691 (1999); Wallace, et al. (Protein Science, vol. 5:1001-1013 (1996); Bone, et al., Biochemistry, vol. 30:10388-10398 (1991); Barth, et al. (1993) Drug Design and Discovery, vol. 10:297-317; Gregory, et al. (1993), Protein Eng., vol. 6, no. 1:29-35; Artymiuk, et al. (1994), J. Mol. Biol., vol. 243:327-344; and Fischer, et al. (1994), Protein Sci., vol. 3:769-778). A particularly preferred approach employs functional site descriptors (FSDs). See U.S. Ser. No. 09/322,067, filed May 27, 1999. Using FSDs, prediction of a protein's biological function requires only an approximation of the three-dimensional orientation of two or more amino acid residues in a region responsible for the particular function of the protein under investigation. Broadly, FSDs define spatial configurations for protein functional sites that correspond with particular biological functions, and it is known that function derives from structure. FSDs provide three-dimensional representations of protein functional sites, for example, ligand binding domains (e.g., domain that bind a ligand, for example, a substrate, a co-factor, or an antigen), protein-protein interaction sites or domains, and enzymatic active sites.

A functional site descriptor typically comprises a set of geometric constraints for one or more atoms in each of two or more amino acid residues comprising a functional site of a protein. Preferably, the atoms are selected from the group consisting of amide nitrogens, α-carbons, carbonyl carbons, and carbonyl oxygens within a polypeptide backbone, β-carbons of amino acid residues, and pseudoatoms, e.g., a side chain center of mass.

The geometric constraints of an FSD preferably are selected from the group consisting of an atomic position specified by a set of three dimensional coordinates, an interatomic distance (or range of interatomic distances), and an interatomic bond angle (or range of interatomic bond angles). When a geometric constraint refers to atomic position, reference is typically made to a set of three-dimensional coordinates. Such constraints can relate to RMSDs. Other geometric constraints concern interatomic distances, preferably interatomic distance ranges, or interatomic bond angles, preferably interatomic bond angle ranges.

In some embodiments, an FSD can also include one or more conformational constraints that refer to the presence of a particular secondary structure, for example, a helix, or location, for example, near the amino or carboxy terminus of a protein. FSDs can be implemented in electronic form, so that they can be used in computerized methods. Typically, functional site descriptors comprising two to about 50 or more geometric constraints can be developed for a particular biological function. In many embodiments, the number of geometric constraints in an FSD is from about 4-25, often from about 5-20.

As indicated above, FSDs can be built for any type of protein function. Functions of particular interest include enzymatic activities. At present, more than 180 different enzymatic activities have been classified, and are listed by enzyme name in the following table. The particular classification of an enzyme listed in the following table is defined in accordance with the enzyme classification system as described in, e.g., Enzyme Nomenclature, NC-IUBMB, Academic Press, New York, N.Y. (1992), and at www.biochem.ucl.ac.uk/bsm/enzymes/index.html.

| E.C. Number | Enzyme Name |
|---|---|
| 1.1.1.2 | Alcohol dehydrogenase (NADP+) |
| 1.1.1.21 | Aldehyde reductase |
| 1.1.1.27 | L-lactate dehydrogenase |
| 1.1.1.28 | D-lactate dehydrogenase |
| 1.1.1.29 | Glycerate dehydrogenase |
| 1.1.1.34 | HMG-CoA reductase |
| 1.1.1.42 | Isocritrate dehydrogenase (NADP+) |
| 1.1.1.49 | Glucose-6-phosphate 1-dehydrogenase |
| 1.1.1.50 | 3-alpha-hydroxysteroid dehydrogenase (B-specific) |
| 1.1.1.53 | 3-alpha(or 20-beta)-hydroxysteroid dehydrogenase |
| 1.1.1.62 | Estradiol 17 beta-dehydrogenase |
| 1.1.1.95 | Phosphoglycerate dehydrogenase |
| 1.1.1.159 | 7-alpha-hyciroxysteroid dehydrogenase |
| 1.1.1.184 | Carbonyl reductase (NADPH) |
| 1.1.1.206 | Tropine dehydrogenase |
| 1.1.1.236 | Tropinone reductase |
| 1.1.1.252 | Tetrahydroxynaphthalene reductase |
| 1.1.3.7 | Aryl-alcohol oxidase |
| 1.1.3.15 | (S)-2-hydroxy-acid oxidase |
| 1.1.99.8 | Alcohol dehydrogenase (acceptor) |
| 1.2.1.2 | Formate dehydrogenase |
| 1.2.1.5 | Aldehyde dehydrogenase (NAD(P)+) |
| 1.2.1.8 | Betaine-aldehyde dehydrogenase |
| 1.2.1.12 | Glyceraldehyde 3-phosphate dehydrogegnase |
| | (phosphorylating) |
| 1.2.3.3 | Pyruvate oxidase |
| 1.3.99.2 | Butyryl-CoA dehydrogenase |
| 1.4.1.2 | Glutamate deydrogeanse |
| 1.4.1.3 | Glutamate dehydrogenase (NAD(P)+) |
| 1.4.3.3 | D-amino acid oxidase |
| 1.4.3.6 | Amine oxidase (copper-containing) |
| 1.5.1.3 | Dihydrofolate reductase |
| 1.6.4.2 | Glutathione reductase (NADPH) |
| 1.6.4.8 | Trypanothione reductase |
| 1.6.99.7 | Dihydropteridine reductase |
| 1.8.1.4 | Dihydrolipoamide dehydrogenase |
| 1.11.1.1 | NADH peroxidase |
| 1.11.1.6 | Catalase |
| 1.11.1.7 | Peroxidase |
| 1.11.1.10 | Chloride peroxidase |
| 1.11.1.11 | L-ascorbate peroxidase |
| 1.14.14.1 | Aromatase |
| 1.14.99.7 | Squalene epoxidase |
| 2.1.1.45 | Thymidylate synthase |
| 2.1.1.60 | Calmodulin |
| 2.1.1.63 | Methylated-DNA--[protein]-cysteine S-methyltransferase |
| 2.1.1.73 | Site-specific DNA-methyltransferase (cytosine-specific) |
| 2.1.2.2 | Phosphorbosylglycinamide formyltransferase |
| 2.1.3.3 | Ornithine carbamoyltransferase |
| 2.2.1.1 | Transketolase |
| 2.3.1.12 | Dihydrolipoamide S-acetyltransferase |
| 2.3.1.28 | Chioramphenicol O-acetyltransferase |
| 2.3.1.39 | [Acyl-carrier protein] 5-malonyltransferase |
| 2.3.1.41 | 3-oxoacyl-[acyl-carrier protein] synthase |
| 2.3.1.61 | Dihydrolipoamide S-succinyltransferase |
| 2.3.2.13 | Protein-glutamine gamma-glutamyltransferase |
| 2.4.1.1 | Phosphorylase |
| 2.4.2.10 | Orotate phosphoribosyltransferase |
| *2.4.2.14 | Amidophosphoribosyltransferase |
| 2.4.2.29 | Queuine tRNA-ribosyltransferase |
| 2.4.2.30 | NAD(+) ADP-ribosyltransferase |
| 2.5.1.1 | Dimethylallyltransferase |
| 2.5.1.7 | UDP-N-acetylglucosamine 1-carboxyvinyltransferase |
| 2.5.1.10 | Geranyltranstransferase |
| 2.5.1.18 | Glutathione transferase |
| *2.6.1.1 | Aspartate aminotransferase |
| *2.6.1.16 | Glucosamine--fructose-6-phosphate |
| | aminotransferase (isomerizin) |
| 2.7.1.11 | 6-phosphofructokinase |
| 2.7.1.21 | Thymidine kinase |
| 2.7.1.30 | Glycerol kinase |
| 2.7.1.37 | Protein kinase |
| 2.7.1.38 | Phosphorylase kinase |
| 2.7.1.40 | Pyruvate kinase |
| 2.7.1.69 | Protein-N(PI)-phosphohistidine-sugar phosphotransferase |
| 2.7.1.105 | 6-phosphofructo-2-kinase |
| 2.7.1.112 | Protein-tyrosine kinase |
| 2.7.1.117 | [Myosin light-chain] kinase |
| 2.7.1.123 | Calcium/calmodulin-dependent protein kinase |
| 2.7.2.3 | Phosphoglycerate kinase |
| 2.7.3.3 | Arginine kinase |
| 2.7.4.6 | Nucleoside-diphosphate kinase |
| 2.7.4.8 | Guanylate kinase |
| 2.7.7.6 | DNA-directed RNA polymerase |
| 2.7.7.7 | DNA-directed DNA polymerase |
| 2.7.7.10 | UTP--heoxe-1-phosphate uridylytransferase |
| 2.7.7.48 | RNA-directed RNA polymerase |
| 2.7.7.49 | RNA-directed DNA polymerase |
| 2.7.7.50 | mRNA guanylyltransferase |
| 2.8.1.1 | Thiosulfate sulfurtransferase |
| 2.8.3.12 | Glutaconate CoA-transferase |
| 3.1.1.1 | Carboxylesterase |
| 3.1.1.3 | Triacylglycerol lipase |
| 3.1.1.4 | Phospholipase A2 |
| 3.1.1.45 | Carboxymethylenebutenolidase |
| 3.1.1.47 | 2-acetyl-1-alkylglycerophosphocholine esterase |
| 3.1.3.2 | Acid phosphatase |
| 3.1.3.11 | Fructose-bisphosphatase |
| 3.1.3.16 | Serine/threonine specific protein phosphatase |
| 3.1.3.46 | Fructose-2,6-bisphosphate 2-phosphatase |
| *3.1.3.48 | Protein-tyrosine-phosphatase |
| 3.1.4.11 | 1-phosphatidylinositol-4,5-bisphosphate phosphodiesterase |
| 3.1.11.2 | Exodeoxyribonuclease III |
| 3.1.21.4 | Type II site-specific deoxyribonuclease |
| 3.1.25.1 | Deoxyribonuclease (pyrimidine dimer) |
| 3.1.26.4 | Ribonuclease H |
| 3.1.27.3 | Ribonuclease T1 |
| 3.1.27.4 | Ribonuclease U2 |
| 3.2.1.1 | Alpha-amylase |
| 3.2.1.2 | Beta-amylase |
| 3.2.1.4 | Cellulase |
| 3.2.1.8 | Endo-1,4-beta-xylanase |
| 3.2.1.14 | Chitinase |
| 3.2.1.17 | Lysozyme |
| 3.2.1.18 | Exo-alpha-sialidase |
| 3.2.1.21 | Beta-glucosidase |
| 3.2.1.23 | Beta-galactosidase |
| 3.2.1.85 | 6-phospho-beta-galactosidase |
| 3.2.1.122 | Alpha glucosidase |
| 3.2.2.1 | Purine nucleosidase |
| 3.2.2.22 | rRNA N-glycosidase |
| 3.4.11.1 | Leucyl aminopeptidase |
| 3.4.11.5 | Prolyl aminopeptidase |
| 3.4.13.19 | Dehydropeptidase I |
| 3.4.16.6 | Carboxypeptidase D |
| 3.4.17.2 | Carboxypeptidase B |
| 3.4.19.3 | Pyroglutamyl-peptidase I |
| 3.4.21.1 | Chymotrypsin |
| 3.4.21.4 | Trypsin |
| 3.4.21.5 | Thrombin |
| 3.4.21.32 | Brachyurin |
| 3.4.21.35 | Tissue kallikrein |
| 3.4.21.62 | Subtilisin |
| 3.4.21.66 | Thermitase |
| 3.4.21.81 | Streptogrisin B |
| 3.4.21.82 | Glutamyl endopeptidase II |
| 3.4.21.88 | Repressor lexA |
| 3.4.22.2 | Papain |
| 3.4.22.28 | Picornain 3C |
| 3.4.23.16 | Retropepsin |
| 3.4.23.20 | Penicillopepsin |
| 3.4.24.27 | Thermolysin |
| 3.4.24.46 | Adamalysin |
| 3.5.1.1 | Asparaginase |
| 3.5.1.5 | Urease |
| 3.5.1.31 | Formylmethionine deformylase |
| 3.5.1.38 | Glutaminase-(asparagin-)ase |
| 3.5.1.59 | N-carbamoylsarcosine amidase |
| 3.5.3.3 | Creatinase |
| 3.5.4.4 | Adenosine deaminase |
| 3.6.1.1 | Inorganic pyrophosphatase |
| 3.6.1.7 | Acylphosphatase |
| 3.6.1.23 | dUTP pyrophosphatase |
| 3.6.1.34 | H(+)-transporting ATP synthase |
| 3.6.1.36 | H/K ATPase |
| 3.6.1.38 | Ca ATPase |
| 3.8.1.5 | Haloalkane dehalogenase |
| 4.1.1.1 | Pyruvate decarboxylase |
| 4.1.1.7 | Benzoylformate decarboxylase |
| 4.1.1.31 | Phosphoenolpyruvate carboxylase |
| 4.1.2.13 | Fructose-biphosphate aldolase |
| 4.1.2.14 | 2-dehydro-3-deoxyphosphogluyconate aldolase |
| 4.1.2.17 | L-fuculose-phosphate aldolase |
| 4.1.3.3 | N-acetylneuraminate lyase |
| 4.1.3.7 | Citrate (si)-synthase |
| 4.2.1.1 | Carbonate dehydratase |
| 4.2.1.2 | Fumarate hydratase |
| 4.2.1.11 | Phosphopyruvate hydratase |
| 4.2.1.24 | Porphobilinogen synthase |
| 4.2.1.39 | Gluconate dehydratase |
| 4.2.1.51 | Prephenate dehydratase |
| 4.2.1.52 | Dihydrodipicolinate synthase |
| 4.2.1.60 | 3-hydroxydecanoy1-[acyl-Carrier protein] dehydratase |
| 4.2.99.18 | DNA-(apurinic or apyrimidinic site) lyase |
| 4.3.2.1 | Argininosuccinate lyase |
| 4.6.1.2 | Guanylate cyclase |
| 5.1.1.7 | Diaminopimelate epimerase |
| 5.1.2.2 | Mandelate racemase |
| 5.3.1.1 | Triosephosphate isomerase |
| 5.3.1.5 | Xylose isomerase |
| 5.3.1.10 | Glucosamine-6-phosphate isomerase |
| 5.3.3.1 | Steroid delta-isomerase |
| 5.3.3.10 | 5-carboxymethyl-2-hydroxymuconate delta-isomerase |
| 5.3.99.3 | prostaglandin endoperoxide synthase |
| 5.4.2.1 | Phosphoglycerate mutase |
| 5.4.2.2 | Phosphoglucomutase |
| 5.4.99.5 | Chorismate mutase |
| 5.5.1.1 | Muconate cycloisomerase |
| 5.99.1.2 | DNA topoisomerase |
| 5.99.1.3 | DNA topoisomerase (ATP-hydrolysing) |
| 6.2.1.5 | Succinate--CoA ligase (ADP-forming) |
| 6.3.4.4 | Adenylosuccinate synthase |
| 6.3.4.14 | Biotin carboxylase |
| 6.3.5.2 | GMP synthase (glutamine-hydrolysing) |
| 6.3.5.5 | Carbamoyl-phosphate synthase (glutamine-hydrolysing) |
| 6.4.1.2 | Acetyl-CoA carboxylase |

As will be appreciated by those in the art, use of the instant invention in conjunction with above FSDs and other three-dimensional templates of protein function, as well as with such other constructs as may be later developed, are within the scope of the invention.

Automated Implementation

The various techniques, methods, and aspects of the instant invention can be implemented in part or in whole using computer-based systems and methods. Additionally, computer-based systems and methods can be used to augment or enhance the functionality described above, increase the speed at which the functions can be performed, and provide additional features and aspects as a part of or in addition to those of the present invention as described herein.

The various embodiments, aspects, and features of the invention described above can be implemented using hardware, software, or a combination thereof, and can be implemented using a computing system having one or more processors. In alternative embodiments, these elements are implemented using a processor-based system capable of carrying out the functionality described with respect thereto. Typically, a computer includes one or more processors. The processor(s) is(are) connected to a communication bus. Various software embodiments are described in terms of this example computer system. The embodiments, features, and functionality of the invention are not dependent on a particular computer system or processor architecture or on a particular operating system, algorithm, or software. In fact, given the instant description, it will be apparent to a person of ordinary skill in the relevant art how to implement the invention using other computer or processor systems and/or architectures.

In some embodiments, a processor-based system can include a main memory, such as a random access memory (RAM), and can also include one or more secondary memories. The secondary memory can include, for example, a hard disk drive and/or a removable storage drive, e.g., a floppy disk drive, a magnetic tape drive, an optical disk drive, etc. The removable storage drive reads from and/or writes to a removable storage medium, such as a floppy disk, magnetic tape, optical disk, etc. that can be read by and/or written to by a removable storage drive. The removable storage media includes a computer usable storage medium having stored therein computer software and/or data. Other alternative embodiments and configurations can also be employed.

A computer system according to the invention can also include a communications interface to allow software and data to be transferred between computer system and external devices. Examples of communications interfaces include modems, network interfaces (such as, for example, an Ethernet card), a communications port, a PCMCIA slot and card, etc. Software and data transferred via communications interface 524 are in the form of signals which can be electronic, electromagnetic, optical, or other signals capable of being received by the communications interface. These signals are provided to the communications interface via a channel that carries signals and can be implemented using a wireless medium, wire or cable, fiber optics, or other communications media.

In this document, the terms “computer program medium” and “computer usable medium” are used to generally refer to media such as a removable storage device, a disk capable of installation in a disk drive, and signals on channels. These computer program products provide software or program instructions to the computer system.

Computer programs (also called computer control logic) can be stored in a memory. Computer programs can also be received via a communications interface. Such computer programs, when executed, enable the computer system to perform the features of the present invention. In particular, the computer programs, when executed, enable the processor(s) to perform the features of the present invention. Accordingly, such computer programs represent controllers of the computer system.

### EXAMPLES

The following examples are provided to illustrate the practice of preferred embodiments of the instant invention, and in no way limit the scope of the invention.

### Example 1

SICHO-Mediated Folding of 8 Representative Proteins

The test set employed in this work is representative of single domain water-soluble proteins⁴⁰ and consists of the following proteins that were previously studied⁶ in the CAPLUS model: the small structured protein fragment of 6 pti, chosen for comparison with the work of Smith-Brown et al., the all-α protein myoglobin (1 mbs), the α/β motifs of protein G, thioredoxin, flavodoxin, and an all-β protein, 1 pcy. In addition, the folding of a 247-residue TIM barrel, Atim, and the β-protein 4 fab was also examined. The set of constraints used in these studies have been reported previously,⁶ but only in those cases where the studied protein is the same. When a smaller number of constraints are used, they were randomly chosen from the larger constraint set. For the two proteins not studied previously, the set of long-range constraints employed appears in Tables II and III, below. The short-range constraints, as before, come from the three-letter code of the DSSP assignment³⁷ of the native secondary structure, and are as described above.

TABLE II
Tertiary Constraint Lists for 4fab

| 27 constraints | | 16 constraints |
|---|---|---|
| 1 PRO | ASN-100 | xx |
| 4 GLN | MET-95 | |
| 8 THR | PRO-107 | xx |
| 8 ILE | PRO-21 | xx |
| 11 ILE | LEU-21 | |
| 11 THR | LEU-107 | |
| 15 ILE | LEU-111 | xx |
| 19 LEU | ALA-109 | xx |
| 20 LYS | SER-79 | xx |
| 23 TRP | SER-40 | |
| 23 PHE | SER-76 | |
| 29 HIS | LEU-98 | xx |
| 34 LYS | GLY-55 | |
| 37 LYS | TYR-55 | |
| 39 TYR | ARG-54 | xx |
| 39 SER | ARG-94 | xx |
| 40 LEU | TRP-52 | xx |
| 44 GLY | LYS-89 | xx |
| 40 LEU | TRP-78 | xx |
| 42 ASP | LEU-87 | |
| 43 VAL | GLN-90 | |
| 53 LEU | ILE-78 | xx |
| 56 PHE | VAL-67 | xx |
| 67 ASP | PHE-87 | |
| 80 LEU | ILE-109 | |
| 92 GLY | PHE-106 | xx |
| 95 TRP | GLN-101 | xx |

TABLE III
Tertiary Constraint Lists for Atim

| Set of 62 | Set of 50 | Set of 37 | Set of 62 | Set of 50 | Set of 37 |
|---|---|---|---|---|---|
| constraints | constraints | constraints | constraints | constraints | constraints |
| 2-228 | xx | xx | 91-125 | xx | 87-120 |
| 4-37 | xx | | 91-231 | xx | 90-122 |
| 4-206 | xx | xx | 93-125 | xx | |
| 6-123 | xx | xx | 94-166 | xx | |
| 6-89 | | | 95-168 | xx | |
| 6-162 | | | 98-126 | xx | xx |
| 7-248 | xx | xx | 98-145 | xx | xx |
| 10-94 | xx | | 105-145 | xx | |
| 11-64 | xx | xx | 105-148 | xx | |
| 11-237 | xx | xx | 109-152 | xx | |
| 15-46 | xx | xx | 112-149 | xx | xx |
| 20-49 | | | 112-161 | xx | xx |
| 23-237 | | 24-54 | 116-153 | xx | 121-160 |
| 27-59 | | | 127-145 | xx | |
| 27-241 | | 32-59 | 127-165 | xx | |
| 30-245 | xx | xx | 128-142 | | |
| 36-58 | xx | xx | 128-165 | xx | xx |
| 26-248 | xx | 41-91 | 130-175 | xx | xx |
| 37-89 | xx | | 133-181 | xx | |
| 39-123 | xx | 44-82 | 142-165 | xx | |
| 47-63 | | | 142-189 | xx | xx |
| 47-87 | | | 143-192 | xx | |
| 51-86 | xx | xx | 150-197 | xx | |
| 59-245 | xx | xx | 155-200 | xx | xx |
| 60-89 | | | 162-208 | xx | xx |
| 63-90 | xx | | 165-189 | xx | xx |
| 66-79 | | 67-111 | 165-209 | xx | xx |
| 68-114 | xx | | 183-225 | xx | xx |
| 79-114 | xx | | 193-205 | xx | xx |
| 89-162 | | 82-120 | 215-244 | xx | xx |
| 90-122 | xx | xx | 230-248 | xx | |

Results of Monte Carlo Simulated Annealing

The results of stage 2 are compiled in Table IV, below. The numbers of constraints are given next to protein PDB codes.¹⁴ An estimate of the cRMSD from the PDB structure and conformational energy (in dimensionless k_{B}T units) is given for the last snapshot of each trajectory. The cRMSD is measured between the Cα's of the real structure and the roughly estimated position of the Cα's of the model chain. The latter are obtained according to the following definition:

R_{αi}^{C}=(4 rᵢ+rᵢ₋₁+Rᵢ₊₁)/6, where the sum in the brackets is over the corresponding side chain coordinates of the model chain. The exact agreement of the secondary structure of the predicted fold and the experimental structure was not examined in detail; however, in all runs, it was very close to the target with a small tendency for extension (by one or two residues) of helical fragments in some cases (e.g., the short helix of plastocyanin). The cRMSD and the energy (in dimensionless k_{B}T units) correspond to the last snapshots of the second simulated thermal annealing runs.

Generally, the predicted structures cluster into two well-defined groups, one of this dominates on the basis of energy, and which is taken to approximate native structure. The remaining, misfolded structures (when observed more than once) were also similar to each other. They represent the topological mirror structure where the chirality of the connections between secondary structural elements (helices and β-strands) is reversed, but the chirality of the secondary structure elements is the same as in the native state, e.g., helices remain right handed. Several interesting observations emerge from the results presented in Table IV, below. First, in the majority of the runs, the native fold is recovered. The accuracy depends on protein size and number of constraints, but only slightly on protein type. Generally, accuracy increases with decreasing protein size. The best accuracy is observed for the 56-residue, B1 domain of protein G,⁴¹ where in most simulations the obtained structures had cRMSD from native below 3 Å. Interestingly, for the smaller 6 pti fragment with a larger number of constraints, the accuracy was systematically somewhat worse. This reflects the effect of protein “regularity.” The fold of protein G has a high content of regular secondary structure, while in the 6 pti fragment, a substantial fraction of the chain is classified as a loop or coil. The analysis of other cases shows a tendency towards higher accuracy for more regular folds. The accuracy of helical and α/β proteins is greater than for all β-proteins. This is clearly demonstrated on comparison of 1 pcy with 2 trx. While both proteins are of comparable size, for 2 trx with 16 constraints, structures with a cRMSD below 3.5 Å are produced, but for 1 pcy with 15 constraints, structures above 5.2 Å result.

TABLE IV
Coordinate cRMSD and Conformational
Energy of the Final Structure at the End of the
Simulated Thermal Annealing Procedure

| Name | Run no. | cRMSD (Å) | Energy |
|---|---|---|---|
| 6pti(18)^{a} | 1 | 3.3^{b} | −321.9 |
| 41 res | 2 | 3.8 | −313.2 |
| (18-56 fragment) | 3 | 4.1 | −302.8 |
| 6pti(9/S1) | 1 | 4.1 | −336.4 |
| | 2 | 4.2 | −345.4 |
| | 3 | 3.6 | −318.9 |
| | 4 | 3.8 | −385.9 |
| 6pti(9/S2) | 1 | 3.8 | −331.8 |
| | 2 | 4.3 | −320.2 |
| | 3 | 4.0 | −341.6 |
| | 4 | 4.4 | −353.6 |
| 6pti(9/S3) | 1 | 3.4 | −303.1 |
| | 2 | 4.0 | −318.7 |
| | 3 | 4.8 | −324.5 |
| | 4 | MI^{c} | −323.2 |
| | 5 | MI | −322.5 |
| 6pti(9/S4) | 1 | MI | −319.1 |
| | 2 | 3.8 | −312.8 |
| | 3 | 4.0 | −320.8 |
| | 4 | 4.2 | −280.4 |
| | 5 | 4.1 | −302.0 |
| 6pti(9/S5) | 1 | 3.9 | −370.0 |
| | 2 | MI | −324.7 |
| | 3 | MI | −283.3 |
| | 4 | 4.4 | −355.2 |
| | 5 | 4.2 | −338.2 |
| 1gb1(8) | 1 | 2.4 | −539.6 |
| 56 res | 2 | 2.6 | −527.7 |
| | 3 | 2.6 | −530.2 |
| | 4 | 2.7 | −562.3 |
| | 5 | 2.7 | −548.0 |
| | 6 | 2.7 | −542.0 |
| | 7 | 3.0 | −550.5 |
| | 8 | 3.2 | −586.7 |
| | 9 | 3.5 | −563.7 |
| | 10 | 4.0 | −551.0 |
| | 11 | MI | −535.3 |
| 1ctf(10) | 1 | 3.4 | −710.5 |
| 68 res | 2 | 3.6 | −758.3 |
| | 3 | 3.7 | −720.9 |
| | 4 | 3.7 | −746.6 |
| | 5 | 4.1 | −622.5 |
| | 6 | MI | −655.1 |
| | 7 | 4.6 | −700.0 |
| | 8 | 3.8 | −692.0 |
| | 9 | 3.2 | −727.2 |
| | 10 | 3.8 | −749.4 |
| 1pcy(46) | 1 | 3.1 | −841.8 |
| 99 res | 2 | 3.6 | −824.5 |
| | 3 | 3.5 | −787.2 |
| | 4 | 3.8 | −783.3 |
| | 5 | 3.9 | −834.7 |
| | 6 | 3.5 | −848.0 |
| | 7 | MI | −744.2 |
| 1pcy(25) | 1 | 4.7 | −944.3 |
| | 2 | 4.8 | −786.7 |
| | 3 | 4.5 | −898.0 |
| | 4 | 5.2 | −928.4 |
| 1pcy(15) | 1 | MI | −870.7 |
| | 2 | 5.6 | −860.8 |
| | 3 | 5.2 | −874.7 |
| | 4 | 5.3 | −925.1 |
| 2trx(16) | 1 | 3.8 | −1098 |
| 108 res | 2 | 3.5 | −1089 |
| | 3 | 4.5 | −1022 |
| 2trx(30) | 1 | 2.8 | −1036 |
| | 2 | 3.2 | −1037 |
| | 3 | 3.7 | −1041 |
| | 4 | MI | −844 |
| 4fab(27) | 1 | 4.5 | −959 |
| 111 res | 2 | 4.4 | −1006 |
| | 3 | 4.9 | −1037 |
| | 4 | 4.1 | −1031 |
| | 5 | MI | −984 |
| 4fab(16) | 1 | 5.0 | −1042 |
| | 2 | 5.1 | −1062 |
| | 3 | 4.8 | −1041 |
| | 4 | 5.5 | −953 |
| | 5 | 4.9 | −1035 |
| | 6 | 5.8 | −1090 |
| | 7 | MI | −1005 |
| | 8 | MI | −1033 |
| | 9 | MI | −1062 |
| 3fxn(35) | 1 | 3.6 | −1441 |
| 138 res | 2 | 3.8 | −1447 |
| | 3 | 3.6 | −1432 |
| | 4 | 3.5 | −1485 |
| | 5 | 4.5 | −1409 |
| | 6 | 3.5 | −1493 |
| | 7 | 4.4 | −1464 |
| | 8 | 4.1 | −1533 |
| | 9 | MI | −1289 |
| 3fxn(20) | 1 | 3.7 | −1447 |
| | 2 | 3.9 | −1464 |
| | 3 | 3.3 | −1511 |
| | 4 | 4.2 | −1515 |
| | 5 | 4.2 | −1503 |
| | 6 | 4.5 | −1499 |
| 1mba(20) | 1 | 3.5 | −1705 |
| 146 res | 2 | 3.7 | −1733 |
| | 3 | 4.1 | −1705 |
| | 4 | 5.6 | −1605 |
| | 5 | 4.2 | −1849 |
| | 6 | 5.0 | −1570 |
| | 7 | 4.1 | −1741 |
| Atim(62) | 1 | 5.0 | −2412 |
| 247 res | 2 | 5.6 | −2357 |
| | 3 | 5.7 | −2417 |
| | 4 | 5.8 | −2491 |
| | 5 | 5.4 | −2499 |
| Atim(50) | 1 | 5.9 | −2428 |
| | 2 | 6.5 | −2507 |
| | 3 | 5.9 | −2540 |
| | 4 | 6.2 | −2509 |
| Atim(36) | 1 | 6.6 | −2469 |
| | 2 | 6.4 | −2599 |
| | 3 | 6.3 | −2558 |
| | 4 | MI | −2593 |
| | 5 | 6.5 | −2526 |
| | 6 | 6.5 | −2643 |

^{a}In parentheses, the number of long-range constraints, S1, S2, . . . S5, various sets of constraints for 18-56 residue 6pti fragment.
^{b}Coordinate root mean square deviation between crystallographic coordinates of Cα's and the approximate positions of the model Cα's calculated as R_{αi}^{C} = (4rᵢ + rᵢ₋₁ + r_{i +1})/6.
^{c}MI, misfolded structure that has satisfied the long-range constraints, generally the topological mirror image fold.

In the above cases, based on the conformational energy of just one (the last in a trajectory) snapshot, it was possible in all cases to identify the proper fold. However, it should be also noted that this very simple criterion may not always work. Indeed, in the case of the fourth set (S4) of long-range constraints for 6 pti, the difference between the energy of a misfolded state and the lowest energy of properly folded states (simulation #3) was marginal. Moreover, the three remaining properly folded conformations have a higher energy than the misfolded one does. Fortunately, for bigger proteins, the situation is different. The energy gap between the proper fold and misfolded states is usually quite large, except for the cases where substantial all of the protein (or particular protein domain) has a β-conformation, and thus has a smallest number of long-range constraints.

The reasons for the apparently lower reliability of the β-protein prediction are complex, and several long-range constraints are required before complext β-type natural proteins can be folded. These proteins have a larger number of building blocks (compare the number of β-strands in an all-β protein with the number of helices in a helical structure of similar size) and, consequently, more complext folds. Thus, the same number of long-range constraints provides relatively less structural information for β-proteins. As a result, the demands on the force field with a given (small) number of constraints are greater. While frequently the proper fold can be identified by choosing the lowest energy final conformation, as happened in the studies reported here, this may not always be the case. Indeed, when the magnitude of the energy fluctuations is larger than the observed energy difference for the final states, a different protocol for the selection of the proper fold is necessary. Such a protocol is described in the next section.

Structure Refinement and Selection of Native Folds

As described above, the lowest-energy final structures from simulated annealing, representing the putative proper fold and the closest competing alternative topology, were subjected to isothermal Monte Carlo runs using the same force field and sets of constraints. The results of these stage 3 runs are summarized in Table V, below.

TABLE V
Compilation of the Results of the Isothermal Simulations

| | | Average | cRMSD (A) | Average | |
|---|---|---|---|---|---|
| Name | Fold | cRMSD | Cα-fit | energy | (S)^{½} (Å) |
| 6pti(9/S1) | NAT^{a} | 3.69 (0.21) | 4.28 (0.29)^{c} | −323.4 | 10.6 |
| 41 res | MI^{b} | Not observed | | | |
| 18-56 | | | | | |
| 6pti(9/S2) | NAT | 3.67 (0.22) | 3.60 (0.11) | −326.1 | 10.6 |
| | MI | Not observed | | | |
| 6pti(9/S3) | NAT | 4.41 (0.12) | 4.23 (0.07) | −325.0 | 9.9 |
| | MI | 8.01 (0.21) | | −301.0 | 10.6 |
| 6pti(9/S4) | NAT | 4.01 (0.29) | 4.05 (0.22) | −327.6 | 10.6 |
| | MI | 8.11 (0.34) | | −309.8 | 9.6 |
| 6pti(9/S5) | NAT | 4.06 (0.24) | 4.27 (0.14) | −349.7 | 10.8 |
| | MI | 8.34 (0.23) | | −318.4 | 10.2 |
| 1gb1(8) | NAT | 3.11 (0.13) | 3.39 (0.14) | −582.9 | 10.9 |
| 56 res | MI | 8.61 (0.13) | | −567.2 | 11.5 |
| 1ctf(10) | NAT | 3.48 (0.25) | 3.21 (0.08) | −699.9 | 11.2 |
| 68 res | MI | 8.68 (0.16) | | −656.9 | 11.7 |
| 1pcy(46) | NAT | 3.44 (0.11) | 3.80 (0.08) | −856.5 | 12.7 |
| 99 res | MI | 11.34 (0.08) | | −796.9 | 12.7 |
| 1 pcy(25) | NAT | 4.87 (0.12) | 4.88 (0.04) | −952.5 | 13.1 |
| | MI | Not observed | | | |
| 1pcy(15) | NAT | 5.27 (0.06) | 5.70 (0.16) | −891.7 | 13.0 |
| | MI | 7.70 (0.08) | | −841.8 | 12.9 |
| 2trx(30) | NAT | 3.63 (0.15) | 3.11 (0.14) | −1013 | 13.0 |
| 108 res | MI | Not observed | | | |
| 2trx(16) | NAT | 3.43 (0.12) | 3.52 (0.06) | −1082 | 13.3 |
| | MI | 11.88 (0.11) | | −888 | 13.2 |
| 4fab(27) | NAT | 4.77 (0.06) | 4.42 (0.07) | −1040 | 13.8 |
| 111 res | MI | 11.49 (0.13) | | −1011 | 13.8 |
| 4fab(16) | NAT | 5.53 (0.08) | 5.92 (0.10) | −1137 | 14.1 |
| | MI | 12.76 (0.09) | | −1033 | 13.8 |
| 3fxn(35) | NAT | 3.91 (0.12) | 4.06 (0.08) | −1514 | 14.3 |
| 138 res | MI | 12.94 (2.33) | | −1311 | 14.3 |
| 3fxn(20) | NAT | 4.44 (0.22) | 4.12 (0.14) | −1401 | 14.3 |
| | MI | Not observed | | | |
| 1mba(20) | NAT | 4.44 (0.23) | 4.34 (0.05) | −1698 | 15.0 |
| 146 res | MI | Not observed | | | |
| Atim(62) | NAT | 5.19 (0.10) | 5.08 (0.11) | −2423 | 17.4 |
| 247 res | MI | Not observed | | | |
| Atim(50) | NAT | 5.77 (0.06) | 5.96 (0.04) | −2483 | 17.7 |
| | MI | Not observed | | | |
| Atim(36) | NAT | 6.66 (0.09) | 6.74 (0.15) | −2622 | 17.9 |
| | MI | 9.97 (0.05) | | −2549 | 17.9 |

^{a}Native structure.
^{b}Misfolded (generally the topological mirror image fold) structure.
^{c}The number in parentheses is the standard deviation of the coordinate root-mean-square distance in Angstroms between the crystallographic and predicted α-carbon traces; see also the legend for Table IV, above.

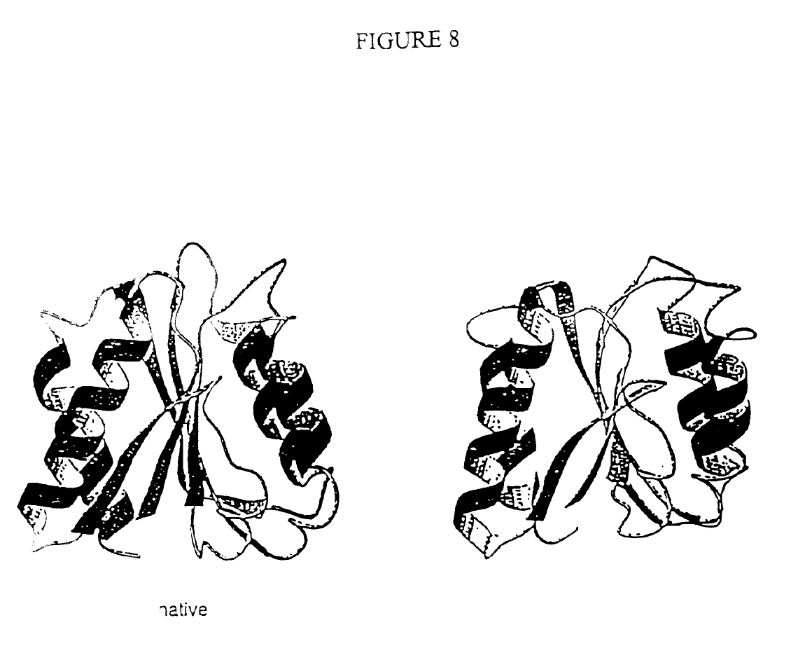

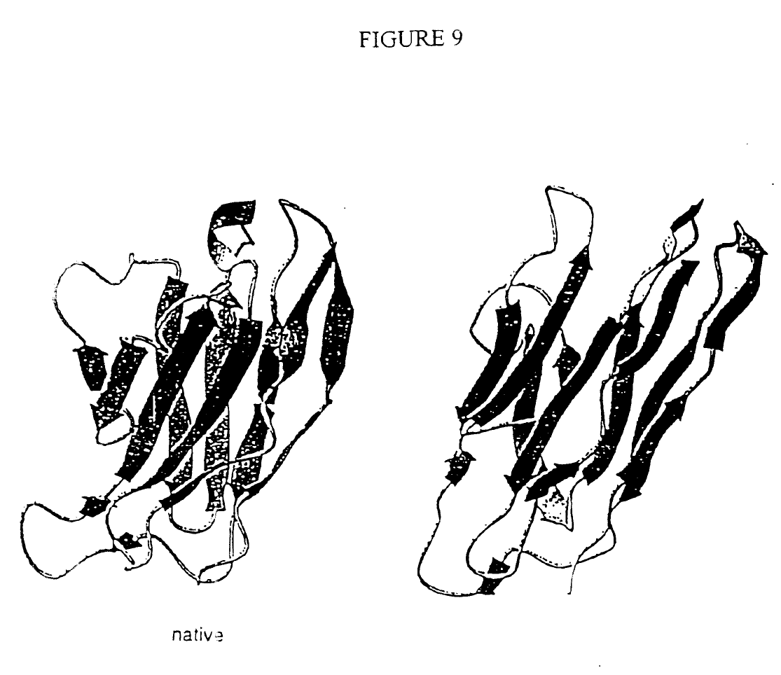

All simulations were done at T=1. The average cRMSD from native and the average energy are computed from 200 snapshots of the Monte Carlo trajectory. In all cases, the proper fold can be identified based on the average conformational energy. Thus, a combination of fast-simulated annealing and long isothermal runs allows the dependable selection of the proper fold. Indeed, during rapid assembly via Monte Carlo-simulated annealing, a fine-tuning of structural details is not always achieved. In long isothermal runs, the misfolded (topological mirror image conformations) states could always be detected as those of higher average conformational energy. For the case 6 pti where five different sets of constraints were examined, the lowest energy misfolded structure has a higher conformational energy than the highest energy proper fold, regardless of the set of constraints. On average, the accuracy of the predicted native fold improved slightly during the isothermal runs and ranges between 3 and 5 Å cRMSD (for the estimated positions of the alpha-carbons), except for the Atim barrel where it was about 6 Å. By way of illustration, FIGS. 8 and 9 present a representative conformation (generated using the MOLMOL⁴² procedure) of 3 fxn and 4 fab obtained from the isothermal refinement runs (employs 20 and 16 constraints, respectively) with a cRMSD of 4.4 Å and 5.5 Å, respectively.

Increasing the number of long-range constraints, on average, leads to some increase in the compactness of the obtained structures, as assessed by their average root-mean-square radius of gyration. There is no obvious systematic difference between the dimensions of the native and misfolded states. Since in both cases the majority of constraints are always satisfied, the difference in conformational energy arises from the underlying force field that has a reasonable level of specificity for nativelike structures. Unfortunately, the non-constraint contributions to the potential are not sufficiently specific to fold the protein (except for a few small proteins) without the assistance of the constraints. On the other hand, within the limit of N/7 constraints, if the constraints are used alone without the remainder of the potential, the resulting structures are essentially random. Thus, it is the synergism of the constraints with the underlying contributions to the potential that permits the folding of these proteins. For some of the test proteins, good folds could be obtained with a smaller than N/7 constraints (e.g., 4 constraints for protein G). The value of N/7 is a conservative estimate of a safe lower bond for all proteins. This number is smaller than required by related methods.⁴⁻⁶

Next, the side-chain-based lattice models serve as targets for building models with two united atoms per residue, e.g., as in the CAPLUS model. Table VI, below, displays the cRMSD data for such reconstructed main chains.

TABLE VI
Comparison of Results for the CAPLUS and SICHO Models With
Exact Secondary and Tertiary Constraints

| | | | | cRMSD in | cRMSD in |
|---|---|---|---|---|---|
| | | | | Å from | Å from |
| PDB | Number of | | Number of | the SICHO | the CAPLUS |
| Name | Residues | Type | Constraints | Model^{a,b} | Model^{a} |
| 1gb1 | 56 | α/β | 8 | 3.4 | 3.3 |
| 1ctf | 68 | α/β | 10 | 3.2 | 4.2 |
| 1pcy | 99 | β | 46 | 3.8 | 3.5 |
| 1pcy | 99 | β | 25 | 4.9 | 5.4 |
| 1pcy | 99 | β | 15 | 5.7 | — |
| 2trx | 108 | α/β | 30 | 3.1 | 3.4 |
| 2trx | 108 | α/β | 16 | 3.5 | — |
| 4fab | 113 | β | 27 | 4.4 | — |
| 4fab | 113 | β | 16 | 5.9 | — |
| 3fxn | 138 | α/β | 35 | 4.1 | 3.9 |
| 3fxn | 138 | α/β | 20 | 4.1 | — |
| 1mba | 146 | α | 20 | 4.3 | 5.9 |
| Atim | 247 | α/β | 62 | 5.1 | — |
| Atim | 247 | α/β | 50 | 6.0 | — |
| Atim | 247 | α/β | 36 | 6.7 | — |

^{a}Average cRMSD of the Cα over an isothermal stability run.
^{b}The average cRMSD is reported from structures obtained after the SICHO model has been mapped into the CAPLUS model and relaxed.

Somewhat surprisingly, there is no significant difference between the average quality of the rebuilt Cα chains and that roughly estimated from a simple linear combination of three successive side chain centers of mass. This shows that the side chain model is consistent with the CAPLUS model used previously. The Cα reconstruction process employed here neglects all the long-range interactions (except of course the target harmonic constraints), and was is done for the sake of computational efficiency.

Comparison with Other Work

As mentioned above, there have been several other attempts to use known secondary structure and some tertiary constraints in the prediction of protein three-dimensional structures. However, the closest studies of other workers who used both known secondary structure and exact tertiary constraints are those of Smith-Brown and coworkers and Aszodi and Taylor. Smith-Brown et al. reported the examination of a number of proteins. By way of example, flavodoxin, a 138 residue α/β protein, was folded to a structure whose backbone cRMSD from native was 3.18 Å for 147 constraints. In contrast, with just 20 constraints, here structures whose cRMSD from native is 4.2 Å were generated. Similarly, for 3 fab, 90 constraints were reportedly required to produce a model whose cRMSD was said to be 4.6 Å. For 4 fab in the present approach, the use of just 27 constraints yielded a model whose cRMSD was 4.4 Å. The reported requirement for a large number of constraints was likely due to the lack of knowledge-based, protein-like background potential.

Another effort to predict the global fold of a protein from a limited number of distance constraints is due to Aszodi et al.⁵ In general, they find that to assemble structures below 5 Å cRMSD, on average, typically more than N/4 constraints are required, where N is the number of residues. Even then, the method reported by Aszodi et al. had problems selecting out the correct fold from competing alternatives. While their best folds are of acceptable accuracy, the competing misfolded structures could be disregarded based on energetic considerations. In contrast, in the simulations presented here, the nativelike fold was easily detected as the lowest energy structure, and just N/7 constraints were required to produce structures of comparable accuracy.

The MONSSTER algorithm uses the CAPLUS model,⁶ and also employs a reduced lattice model of protein, a background, knowledge-based force field, and a simulated thermal annealing Monte Carlo procedure for fold assembly. Using MONSSTER, about N/4 constraints are required to assemble β-type and α/β-proteins, while helical proteins required N/7 constraints. Here, for a representative set of proteins, all types of folds can be assembled with knowledge of, on average, N/7 tertiary constraints. In addition, the results are less sensitive to the distribution of constraints. For example, in the case of the 18-55 fragments of 6 pti in the CAPLUS model, the cRMSD was about 6-8 Å for the different sets of constraints. In contrast, in the side-chain-based model, for all sets of examined constraints, it is about 4 Å. Furthermore, as evidenced by the ability to fold the 247-residue TIM from a fully extended state, much larger systems can be treated. With an increasing number of long-range constraints, the accuracy of assembled structures increases and is consistently better than for previously reported methods. In addition, the resulting models are found to be less sensitive to the constraint distribution.

The instant invention also offers the advantage of speed. For small proteins, the algorithm is essentially interactive. It takes about 5-10 minutes of CPU time on a contemporary workstation to assemble the relatively complex motif of a 68-residue 1 ctf fragment. Since the cost scales approximately as N³, assembly of larger structures requires more time. Thus, a myoglobin folding simulation requires about 2 hours of CPU time.

CONCLUSION

This example demonstrates that the invention provides a powerful new model for the assembly of three-dimensional protein structures from known secondary structure and a small number of tertiary constraints. While the model only explicitly considers side chain centers of mass of the amino acid residues comprising the protein being studied, the effect of backbone atoms is implicitly built into the model force field, which also exploits the structural regularities seen in protein structures. Thus, the invention is fully compatible with more complex models that employ a larger number of united atoms per residue. In all respects, the invention compares favorably with previous approaches having a similar goal: the assembly of tertiary structure from loosely encoded secondary structural biases and a small number of tertiary constraints. Important aspects of the invention include its utilization of side chain center of mass representations and the very small number of tertiary constraints required to assemble moderate resolution folds. For a representative set of al types of single domain proteins (all-α, all-β, α/β motifs), the required number of constraints is about N/7, with N the number of residues in the protein. Furthermore, due to a new, rapid treatment of side chain burial, the invention is applicable to multi-domain proteins.

The invention also provides a relatively simple and reliable protocol of detecting a proper fold from less frequently generated misfolded structures. These misfolded structures are almost exclusively the topological mirror images of the proper fold. In all cases examined to date, the native-like structure always has a lower conformational energy. This and the small number of required tertiary constraints suggest that the invention's underlying force field captures a number of the essential aspects of protein interactions. At the same time, the model is simpler and computationally more efficient than previously employed lattice models.^{6,39} Due to a much lower computational cost (at least one order of magnitude), it is possible to assemble larger structures, including the 247-residue Atim domain.

Finally, using the invention it is also possible to generate at least low resolution folds using only a small set of probable side chain contacts³⁵ (predicted via correlated mutations analysis⁴³) and somewhat more elaborate potentials describing short-range interactions (derived from geometrical analysis of sequentially similar protein fragments). Several example structures such as myohemerythrin (1 hmd) and the complex β-type motif immunoglobulin fold (1 fna), have been assembled.

### Example 2

This example also describes the generation and refinement of protein molecular models. Threading-based target-template alignments were obtained from one standard threading method;¹⁵ but in principle, any could be used.²⁶ The modeling technique employed was SICHO, which employs a very simple, and computationally very efficient, yet quite accurate, representation of protein structure and dynamics.^{17,19} For the purpose of the this application, the model was refined by incorporating evolutionary information into the interaction scheme. Starting from an initial conformation of the model lattice chain that approximately followed the threading template, a Monte Carlo annealing procedure found a conformation that maintained some (but not all) features of the original template and at the same time optimized packing and intra-protein interactions, as defined by the reduced model of the probe protein. This could be also visualized as a folding simulation in a soft tube built around the threading template.

Here, the method was applied to 12 target/template protein pairs that produce various quality models. The parameters of the lattice model force field (more precisely, the balance between the intrinsic force field and the template-related biases) were adjusted by a trial and error method for three of the 12 target/template protein pairs. The obtained parameters were subsequently used in the other 9 simulations. As will become apparent after analysis of the simulation results, the obtained models for the three proteins used for timing the potential were among the best. This may suggest that the method was strongly tuned to these three examples. This was not the case. First, the three proteins belonged to completely different structural classes, so any tuning was rather general, i.e., applicable to the majority of single domain proteins. Second, when the tuning procedure was performed on just a single case (the plastocyanin/azurin pair), almost the same results are obtained, suggesting that the optimal balance between the template-related soft restraints and the intrinsic force field of the model was similar for various proteins. Finally, the poorer results obtained for most of the remaining nine test proteins were simply due to the poor quality of the initial threading models.

The remainder of this example can be outlined as follows. In Methods, the reduced lattice protein model is described, with the protein representation, the model of stochastic dynamics, the interaction scheme, and the template related biases and restraints being discussed. Then, in the Results section, the molecular models obtained from Monte Carlo simulated annealing and subsequent refinement procedures are compared with the initial crude, threading-based models. In the Discussion, the improved models are analyzed to identify typical underlying structural rearrangements.. Certain technical details are found in the Appendix.

Methods

Lattice Model

The reduced modeling of protein structure and dynamics usually employs an alpha carbon main chain representation.^{18,25} Side chains are either completely neglected or treated at various levels of simplification. The choice of the alpha carbon representation is mostly motivated by the high level of geometric regularity of the main chains in folded proteins.²⁵ On the other hand, the packing and interactions between the side chains are perhaps much more sequence specific than are those of the main chain. The latter are very similar in all proteins.

As demonstrated in Example 1, SICHO is a useful protein-modeling tool, as it incorporates many protein-like features, including local conformational propensities and the characteristic packing regularities of protein side chains. A major advantage of SICHO is that the entire conformational space of quite large proteins can be efficiently sampled. For example, with the help of a properly designed force field, loose knowledge of the secondary structure and a few long-range side chain contacts (about N/7, where N is the number of residues), which may come from sparse NMR data or other experimental techniques, low-resolution protein structures can be reproducibly and rapidly assembled for proteins containing up to 250 amino acids or more.

The SICHO model employed in this example is very similar to that used in Example 1, although there are some differences in the protein representation that slightly increase the geometric fidelity of the model.

Reduced Representation of Polypeptide Chains

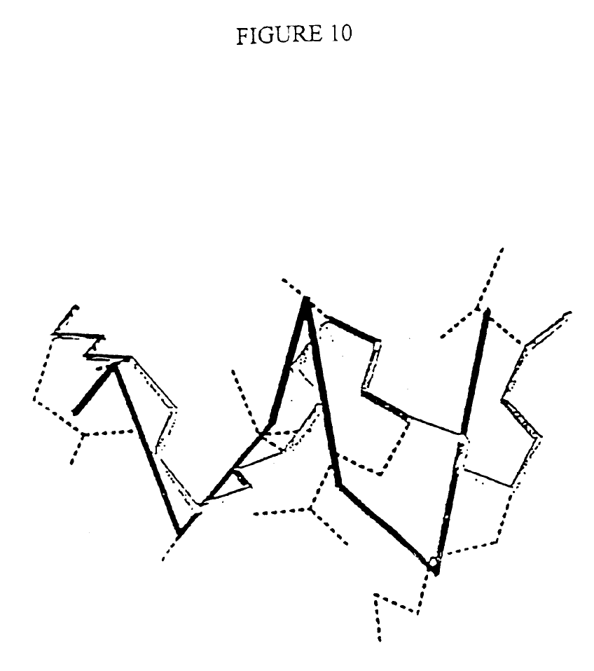

The model chain consists of a string of virtual bonds connecting the interaction centers that correspond to the center of mass of the side chains and the backbone alpha carbons. All heavy atoms have the same weight in this averaging. Thus, the center of glycine coincides with its C_{α}, the center of alanine is located in the middle of the C_{α}-C_{β} bond, the center of valine roughly coincides with the C_{β} atom, etc. These interaction centers (beads) were projected onto an underlying cubic lattice with a lattice spacing of 1.45 Å. This constant defines the spatial resolution of the model. Obviously, the virtual bonds resulting from such a projection are of various lengths, depending on the identity of the two corresponding residues, the main chain conformation and the rotameric state of the side chain (see FIG. 10). A change in any of these variables may change the corresponding virtual bonds (the chain vectors v). In proteins, these distances have a quite broad distribution, ranging from 3.8 Å for a pair of glycines to about 10 Å for some pairs of large side chains in their anti-parallel orientation and expanded conformations. The corresponding set of lattice vectors covers this distribution with good fidelity. The shortest vectors were of the form of (±2, ±2, ±1) or (±3,0,0) vectors, including all possible permutations. The length of these vectors corresponded to a distance of 4.35 Å. The longest lattice vectors were of the (±5, ±2, ±1) type and their length corresponded to 7.94 Å. Thus, the wings of the distribution are cut off. This should not have any noticeable effect on the model's fidelity because the small distance cut-off error is well below the resolution of the model, and the long-distance cut-off error is not important due to very rare occurrences of distances above 8 Å. As a result, the set of allowed lattice bonds consists of 646 vectors, and sequentially adjacent vectors could not be identical.

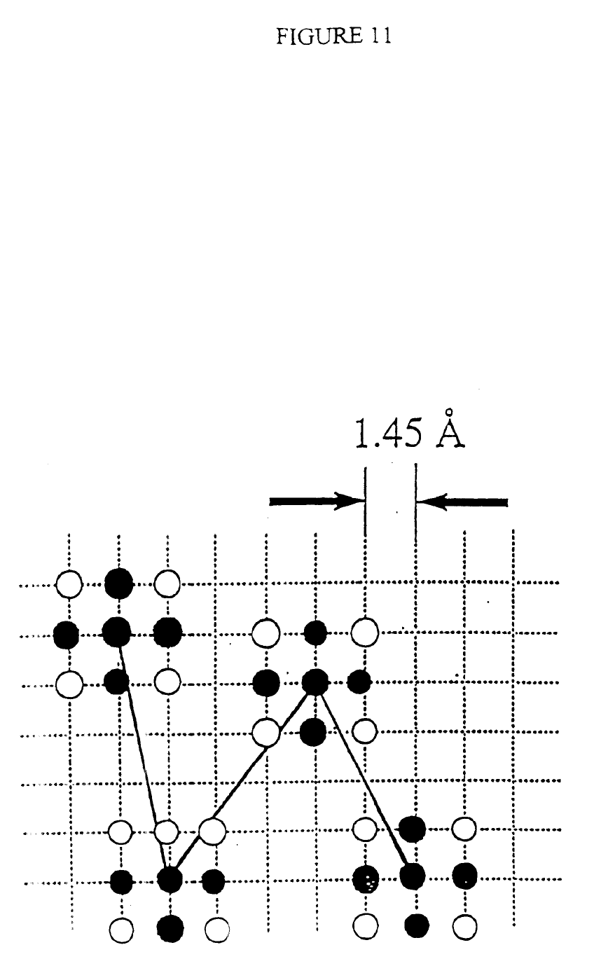

A cluster of excluded volume points was associated with each bead of the model chain. Each cluster consisted of 19 lattice points: the central one; six points at positions (±1,0,0), (0,±1,0) and (0,0,±1) with respect to the central one; and 12 points at positions (±1,±1,0), including all permutations. Thus, the closest approach positions of another cluster with respect to a given cluster were of the form (±2,±2,±1) and (±3,0,0), as measured between the cluster centers. It could be easily calculated that, here, there were 30 closest approach positions. The distance of the closest approaches nicely corresponded to the smallest values of the inter-residue distances in real proteins. Since the average “contact distances” (see the following sections) of the model residues were somewhat larger than the distance of the closest approach, there were many more than 30 spatial orientations of two residues being in contact. Consequently, such a representation of protein structure avoided various anisotropy effects typically seen in the lower resolution lattice protein models. FIG. 11 shows a small fragment of the model chain confined to the underlying cubic lattice with a lattice spacing equal to 1.45 Å. The excluded volume points are denoted by the solid and open circles. The solid circles indicate the three lattice points along the direction orthogonal to the plane of the figure: one in the plane below and one in front of the plane. The open circles denote points in the plane. With the above geometric restrictions, all PDB structures³ could be represented with an average root mean square deviation (RMSD) of about 0.8 Å. Again, the accuracy of the fit does not show any systematic dependence on protein length nor on the orientation of the crystallographic structure with respect to the lattice coordinate system. Some features of the model chain are illustrated in FIG. 10.

Conformational Updating

The simplicity of the model protein representation facilitated the very rapid sampling of conformational space. The Monte Carlo algorithm employs three types of conformational transitions. The first type is a single bead, two-chain vector move. A random displacement of a randomly selected bead is generated and approved provided that the vector lengths and the excluded volume are not violated. The range of a random displacement is from 1 to 5½ lattice units. When accepted by the Metropolis criterion⁴ (see the next section), such a move is equivalent to a collective rearrangement of the main chain and/or the side chain internal coordinates in a real polypeptide chain. The force field of the model, especially its generic components, prevented the acceptance of nonsensical, non protein-like conformations.¹⁷ The second type of motion involved the permutation of three chain vectors. This was a larger scale move that was relatively rarely accepted due to possible steric interactions. The last type of move involved a randomly selected fragment consisting of several chain units. This fragment moved as a rigid body due to appropriate small changes in the two flanking chain vectors. For instance, such a move could translate a helical segment by a small distance, thereby slightly changing the conformation of the corresponding turn or loop regions.

Interaction Scheme

The model force field consisted of several types of potentials. The first were generic biases that penalize against non protein-like conformations. These potentials were sequence independent. Sequence specific contributions to the force field consisted of knowledge-based two-body and multi-body potentials extracted from a statistical analysis of known protein structures. Finally, there were two kinds of potentials that contained evolutionary information extracted from multiple sequence alignments. In all cases, all PDB structures whose sequences were similar to the query sequence have been removed from the structural database used in the derivation of the potential (greater than 25% sequence identity).

1. The Generic Protein Stiffness Potential and Secondary Structure Bias

As defined above, the model chain was intrinsically very flexible. A substantial fraction of its conformations that were allowed due to the assumed simplified hard core interactions did not correspond to any real polypeptide chain conformation. In particular, proteins are relatively stiff polymers. Moreover, folded proteins have very characteristic distributions of certain short-range distances. For example, the bimodal distribution of the distances between the i−th and i+4^{th} th residues reflects the tendency to adopt either of two types of conformations. These correspond to extended (β-type or extended coil) or very compact conformations (as within helices or turns). Such generic features need to be included in the model. Here, the SICHO model differs from that used in Example 1 due to the refined protein representation (a larger number of allowed chain vectors and a modified position of the center of interaction, that also included alpha carbons).

First, for all possible two-vector sequences of the model chain, a direction w was defined that was almost perpendicular to the plane formed by the fragment. A small systematic deviation from the exactly orthogonal direction was introduced in w to obtain vectors that were, on average, parallel to the helix axis and which also accounted for the average supertwist of β-strands.

uᵢ=(vᵢ₋₁\mathring{x}vᵢ−vᵢ₋₁−vᵢ) (1)

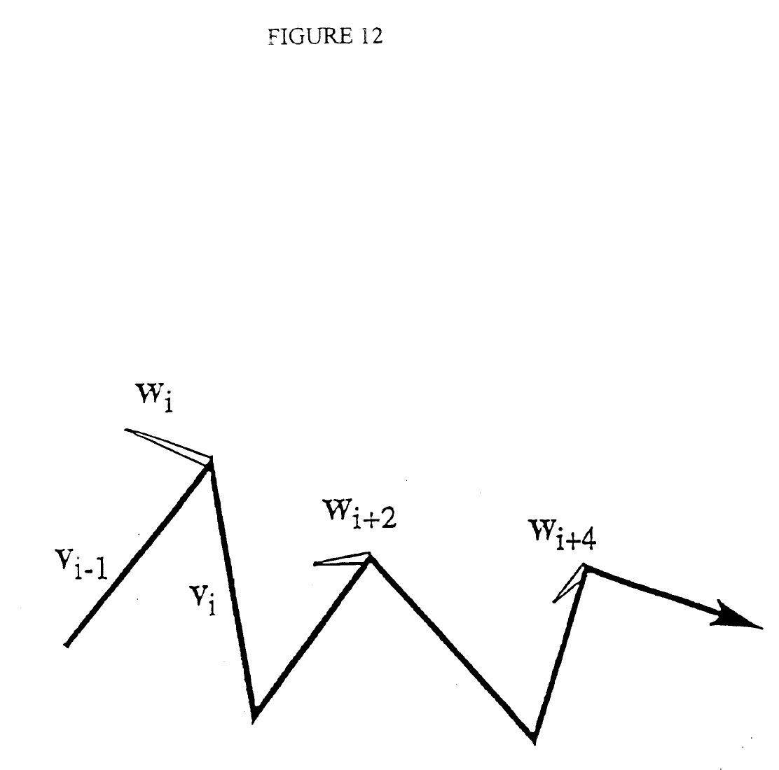

wᵢ=uᵢ/|uᵢ| (2)

where vᵢ is the i−th vector (or virtual bond) of the model chain, the symbol “\mathring{x}” denotes the vector cross product and |uᵢ| is the length of vector uᵢ. Consequently, these “directions of secondary structure” (the vectors w point along a helix or across a β-sheet) were normalized so that their length was equal to 1. The idea is explained in FIG. 12, where the model chain virtual bonds are shown in solid lines and the vectors wᵢ are shown in open arrows.

The stiffness/secondary structure bias has the following form:

$$\begin{matrix}{E}_{stiff} = - {ϵ}_{gen} [ Σmin \{ 0.5 , max ( 0 , {w}_{i} \cdot {w}_{i - 4} ) \} ] \\ - {ϵ}_{gen} [ Σmin \{ 0.5 , max ( 0 , {w}_{i} \cdot {w}_{i - 4} ) \} ]\end{matrix} \qquad ( 3 )$$

ε_{gen} is a constant energy parameter, common for all generic potentials, and Σ means summation along the chain. The above formulation means that the system is energetically stabilized when pairs of “direction of secondary structure” vectors are parallel (positive dot product). As can be read from the above equation, the stabilization energy increased in the range between 90° and 30° (angle between appropriate vectors w) and then maintained its extreme value. Thus, small fluctuations of the secondary structure had no influence on the value of this potential, nor did the changes outside the regular conformations (negative values of the dot product) have any effect on the conformational energy. The minimum value of the stiffness function per residue was equal to ε_{gen}, and the maximum was 0.

Additionally, a bias was introduced towards the specific geometry of helical and β-type expanded states (however, it was quite permissively defined). All conformations were, of course, allowed; the purpose of this bias was to mimic a protein-like (average) distribution of local conformations. Symbolically, this could be written as follows:

E_{struct}Σ{δH1(i)+δH2(i)+δE1(i)+δE2(i)} (4)

with:

$$\begin{matrix}\begin{matrix}δ H1 ( i ) = - {ϵ}_{gen} , & for {r}_{i , i + 4}^{2} < 36 and ( {v}_{i} \cdot {v}_{i + 3} ) > 0 and ( {v}_{i} \cdot {v}_{i + 2} ) < - 5 \\ 0 , & otherwise\end{matrix} & \text{(4a)} \\ \begin{matrix}δ H2 ( i ) = - {ϵ}_{gen} , & for {r}_{i , i + 4}^{2} < 36 and ( {v}_{i} \cdot {v}_{i + 3} ) > 0 and ( {v}_{i} \cdot {v}_{i + 2} ) < - 5 \\ 0 , & otherwise\end{matrix} & \text{(4b)} \\ \begin{matrix}δ E1 ( i ) = - {ϵ}_{gen} , & for 56 < {r}_{i , i + 4}^{2} < 135 and ( {v}_{i} \cdot {v}_{i + 2} ) > 5 \\ 0 , & otherwise\end{matrix} & \text{(4c)} \\ \begin{matrix}δ E2 ( i ) = - {ϵ}_{gen} , & for 56 < {r}_{i , i + 4}^{2} < 135 and ( {v}_{i + 1} \cdot {v}_{i + 3} ) > 5 \\ 0 , & otherwise\end{matrix} & \text{(4d)}\end{matrix}$$

The numerical values are in lattice units and were selected to define a broad range of helical/turn conformations (for the δH1 and δH2 contributions) or expanded conformations (for the δE1 and δE2 contributions). Due to the exclusive character of the two subsets of geometrical conditions for specific chain conformations, the minimum contribution from a residue is equal to −2ε_{gen} (either the first two conditions or the two last conditions can be simultaneously satisfied). Expressed differently, equation (4d) indicated that the system gained an energy equal to −ε_{gen} for being in an expanded β-type conformation. For a four-vector fragment of the chain, this required that the distance between the i−th and i+4^{th} beads (the centers of mass of the side chain plus Cα units) had to lie between 10.7 and 16.8 Å, and the chain vectors vᵢ₊₁ and vᵢ₊₃ have to be oriented in a parallel-like fashion (the dot product>5). Additional stabilization is gained when, for the same fragment, another pair of vectors is parallel (see equation 4c). The broad ranges allowed for substantial fluctuations (without an energetic penalty) around an ideal expanded state and accommodated the variations of the model chain geometry caused by differences in side chain size.

Computational experiments have also been performed where all interactions, except the ones defined above, were turned off. At low temperature, the model chain formed rapidly fluctuating local clusters of expanded and helix-like states. The persistence length and the distributions of the short-range distances along the chains mimicked protein-like geometry.

2. Generic Packing Cooperativity

Two terms were introduced to enforce some of the most general regularities of the dense packing of protein structures.¹⁰ In all the more regular elements of secondary structure (within helices and β-sheets, but not between helices) and, to a lesser extent, in some coil-type fragments and turns, given a contact between a pair of reference residues, there was a very strong preference to have contacts (we provide precise definition of the “contact” later) between the preceding and the following residues. Indeed, the contact maps of globular proteins contain very characteristic strips.²² Those near the diagonal corresponded to the intrahelical contacts, those farther from the diagonal (parallel or antiparallel to the diagonal) correspond to contacts between β-strands within β-sheets. Thus, the following energetic bias was introduced towards such a mode of packing:

$${E}_{map} = - {ϵ}_{gen} \{ \sum \sum ( {δ}_{i , j} \cdot {δ}_{i + 1 , j + 1} \cdot {δ}_{i - 1 , j - 1} ) {δ}_{par} + \sum \sum ( {δ}_{i , j} \cdot {δ}_{i - 1 , j + 1} \cdot {δ}_{i + 1 , j - 1} ) {δ}_{apar} \} \qquad ( 5 )$$

where the summations are over all pairs of residues i,j, and δᵢⱼ is equal to 1 (0) when residues i and j were (were not) in contact. δₚₐᵣ was equal to 1 only when the corresponding chain fragments are oriented in a parallel manner, i.e., when the chain vectors satisfied the following condition (vᵢ₋₁+vᵢ)·(vⱼ₋₁+vⱼ)>0; otherwise, δₚₐᵣ=0. Similarly, δₐₚₐᵣ was equal to 1 when the chain fragments were antiparallel, and it was equal to zero otherwise. For a given contact of a pair of residues, the maximal energetic stabilization due to regular side chain packing was therefore equal to −ε_{gen}, which had the same value as in the previously defined potentials.

The packing cooperativity of the model protein was further enhanced by a term that mimics main chain hydrogen bonds. The geometry of protein hydrogen bonds was translated into a specific range of the model chain geometry. First, a vector was defined that was likely to connect the model beads within motifs that represent regular secondary structure elements. Such a vector should connect beads i and i+3 in a helix and the appropriate beads in a β-sheet. An optimization procedure leads to the following definition of this vector:

hᵢ=3.3(vᵢ₋₁\mathring{x}vᵢ)/|(vᵢ₋₁\mathring{x}vᵢ)|−vᵢ₋₁/|v_{i-1|} (6)

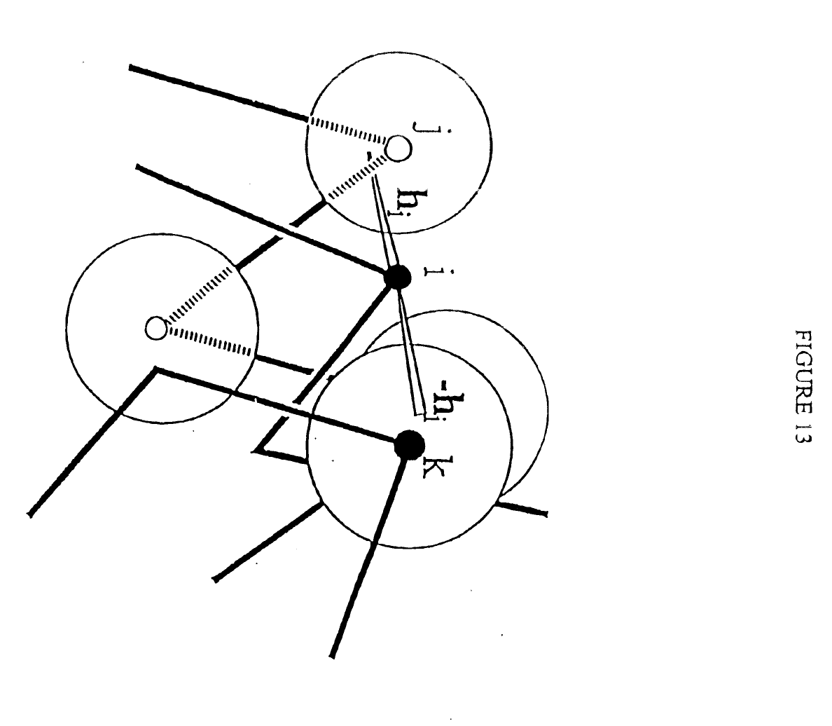

The value of the 3.3 pre-factor has been found to be optimal (or more precisely near optimal) for reproducing the internal main chain hydrogen bonding in the lattice projected PDB structures. However, due to the wide distribution of the model chain bond lengths, there were always some hydrogen bonds that were missed in the model. The coordinates of the vectors hᵢ were rounded-off to the nearest integer value. Thus, in a helix the hᵢ vectors have a component whose length was about 3 lattice units in the direction perpendicular to the three-residue plane (the first term in the above sum) and were also tilted back by a lattice unit (the last term of equation 6). The projection along the helix axis was also about 3 lattice units; this nicely coincided with the 1.5 Å longitudinal increment per residue in a real helix. Residue i was considered to be hydrogen bonded with residue j when the vector hᵢ pointed to any of the 19 points of the excluded volume cluster of residue j. Correspondingly, the vector −hᵢ may point to another cluster. Such a situation was illustrated in FIG. 13, where residue i is hydrogen bonded with residues j and k because the hydrogen bond vectors coincide with the excluded volume of both residues. The excluded volume clusters were symbolically represented by open spheres. Since the excluded volume clusters never overlapped, the maximum number of these “hydrogen bonds” originating from residue i was equal to 2. The total energy of the “hydrogen bond network” could be written as:

E_{H-bond}=−ε_{H-bond}Σ(δ⁺+δ⁻+δ^{+,-} (7)

where δ⁻(δ⁻) equaled 1 when the vector hᵢ(−hᵢ) connected with an excluded volume cluster, and δ^{+,-}=1 when the both vectors connected to some clusters, respectively. Otherwise, the corresponding terms were equal to zero. The cooperative contribution, δ^{+,-}, corresponded to local saturation of the hydrogen bond network.

Again, a computational experiment was done to check the effect of these generic potentials on the behavior of the model system. When only the interactions outlined up to this point were included (all the above short- and long-range generic potentials), the model lacked sequence specific information. At sufficiently low temperatures, the chain adopted either of the following two types of structures, a long (sometimes broken) helical structure or a β-sheet with a right-handed supertwist. These motifs fluctuated and were not structurally unique. In a long chain, these two classes of secondary structure elements sometimes formed separate domains.

3. Sequence Specific Short-Range Interactions

For the sequence of interest, from the structural database, one may extract the statistics of distances between a pair of amino acids (with their interaction centers as defined in the model) Aᵢ and Bᵢ₋ₖ, where A and B denote the identities of the amino acids and i is the position in the chain. Here, k=1, 2, 3, 4, 6 and 8 was considered. The terms for k=3 and k=6 were treated as chiral variables. This meant that the distance between Aᵢ and Bᵢ₊₃ was stored as a positive or negative number, depending on the handedness of the corresponding three-bond segment. For the k=6 case, the chirality was defined for three subsequent supervectors (the doublet of vectors between beads i and i+2, i+2 and i+4, and from i+4 to i+6). As was done here, the sequence of interest could be divided into overlapping short fragments. These could be aligned to the sequences of known structures. The highest scoring fragments provided a set of structural templates. The obtained statistics could be related to a random distribution and the statistical potential of mean force could be appropriately derived. Terms for k=1, 2, 3, and 4 were weighted equally, while the terms for k=6 and k=8 had weights reduced by a factor of two, with respect to lower order terms. Homologous proteins were always excised from the structural database for the purpose of these test calculations. As previously shown, this type of potential very nicely reproduces the local conformational propensities of globular proteins.¹⁷

The short-range potentials could be made even more sequence specific when evolutionary information encoded in homologous sequences was employed. In such a case, the aligned fragments of highly homologous sequences (from the sequence database) were treated as the original test sequence, thereby increasing the strength of the statistics. The details of the derivation procedure are given in Appendix 1.

4. Sequence Specific Pairwise Interactions

The pairwise interactions between model residues were defined by contact potentials in the form of a square well function.

∞, for rᵢⱼ<3

Eᵢⱼ=E^{rep}, for 3≦rᵢⱼ<Rᵢⱼ^{rep}

εᵢⱼ, for Rᵢⱼ^{rep}≦r_{ig}<R_{ig}

0. for R_{ig}<r_{ig} (8)

where εᵢⱼ were the pairwise interaction parameters, r_{ig} was the distance between chain beads i and j, E^{rep}=3 kT was a constant repulsive term operating at very short distances, and Rᵢⱼ^{rep} and R_{ig} were the cut-off values that depend on amino acid type. The values of these cut-off parameters were provided in Table VII.

TABLE VII
Compilation of pairwise cut-off distances for pairwise interactions

| Aᵢ | Aⱼ | Rᵢⱼ^{rep} (Å) | R_{ig} (Å) |
|---|---|---|---|
| Small^{b} | Small | 4.3^{b} | 5.97 |
| Large^{c} | Large | 4.83 | 6.80 |
| Other | Combinations^{d} | 4.57 | 6.32 |
| ^{a}Small amino acids are: Gly, Ala, Ser, Cys. | | | |
| ^{b}This value corresponds to the excluded volume radius of three lattice units; therefore, for pairs of | | | |
| # small amino acids, the soft-core envelope does not exist. | | | |

^{c}Large amino acids. are Phe, Tyr, Trp.
^{d}Small-large, other (than small or large)-large, other-small

The interaction parameters depended not only on amino acid identity, but also on their positions in the polypeptide chain because the derivation of the potentials also used evolutionary information. A more detailed description of the derivation of these potentials is found elsewhere.¹⁸ The total energy contribution from the pairwise interactions was therefore calculated as follows:

Eₚₐᵢᵣ=ΣΣEᵢⱼ (9)

where the summations were over all j>i pairs of residues.

5. Multibody Potentials

The hydrophobic interactions in this model were partially accounted for by pairwise interactions between residues; however, this was not sufficient to generate well packed proteins. Thus, a surface exposure based statistical potential was developed according to the following scheme: Each model residue was assigned 24 surface contact points. A specific subset of these contact points became occupied upon contact with other residues. The main chain Cα atoms contributed separately to the coverage of a given residue. The positions of the Cα atom could be quite well approximated given the positions of three consecutive side chain beads.¹⁷ Some contact points could be multiply occupied. The fraction of non-occupied surface points defined the exposed fraction of a given side chain. Potentials could be derived from a statistical analysis of the protein structures for which the solvent exposure had been determined on the atomic level. The total surface energy was computed as follows:

E_{surface}=ΣE_{b}(Aᵢ, aᵢ) (10)

where aᵢ was the covered fraction of the residue Aᵢ and E_{b}(Aᵢ, aᵢ) was the statistical potential when amino acid type A had aᵢ of its surface points occupied, i.e., the covered fraction of its surface was equal to aᵢ/24.

Studying the distribution of inter-residue contacts in globular proteins, various amino acids have been found to have different tendencies to pack in a parallel or antiparallel fashion. A contact between residues i and j was considered to be “parallel” when (vᵢ₋₁-vᵢ)·(vⱼ₋₁-vⱼ)>0, and “antiparallel” otherwise. Moreover, for a given residue there were strong correlations between the number of parallel and antiparallel contacts given the total number of contacts. Due to the reduced character of this model, the other contributions to the force field did not properly account for such effects. Therefore, the model force field was supplemented by the following multibody potential:

Eₘᵤₗₜᵢ=ΣEₘ(A,nₚ,nₐ) (11)

where Eₘ(A,nₚ,nₐ) was the value of the statistical potential for residue type A having nₚ parallel and nₐ antiparallel contacts. The reference state was a random distribution of contacts. The values along particular diagonals (nₚ+nₐ=n_{c}) were normalized such that the lowest energy for a diagonal was exactly equal to the value of statistical potentials derived from the distribution of the total number of contacts n_{c} for a given type of residue.

6. Total Intrinsic Conformational Energy

The total internal conformational energy of the model chain was equal to:

Eₜₒₜₐₗ=E_{stiff}+Eₘₐₚ+0.875E_{H-bond}+0.75Eₛₕₒᵣₜ+1.25Eₚₐᵢᵣ+0.5E_{surface}+0.5Eₘᵤₗₜᵢ (12)

with the value of generic parameter ε_{gen}=1 kT.

The relative scaling of various potentials was adjusted by a trial and error method in ab initio folding experiments performed for a few selected small proteins, 1 fna, the B domain of protein A and the B1 domain of protein G. The objective was to maintain low secondary structure content in the random coiled state and dense packing with a proper level of secondary structure in the collapsed globular state. For instance, the small 56-residue α/βprotein G domain folded ab initio in about 30% of simulated annealing Monte Carlo simulations to a native-like structure with an RMSD from native in the range of 4 Å. The majority of the remaining misfolded conformations had native-like secondary structures, but they had topological errors, usually involving the wrong order of β-strands in the four-member β-sheet. The model is not sensitive to small variations in these scaling parameters.

Building the Starting Lattice Model

A separate algorithm was used to build an initial lattice model from a given target sequence alignment to a template structure. Such alignments contain gaps and insertions. First, interaction centers were computed from the template. Then, starting from the first aligned position, the lattice chain was sequentially built. At each step in the aligned region, the new vectors were selected so as to minimize the distance of the lattice chain from the equivalent template points. In the gap regions, the distance from the last residue of the preceding aligned fragment to the first residue of the next was divided to generate a set of checkpoints. The number of these checkpoints was equal to the number of target sequence residues that had to be mounted to span the gap. The checkpoints outside the entire alignment were generated in a random fashion. The set of all checkpoints provides the target for the starting lattice model. The model chain maintains the excluded volume and satisfies the other geometric restrictions discussed before.

Implementation of the Template Restraints

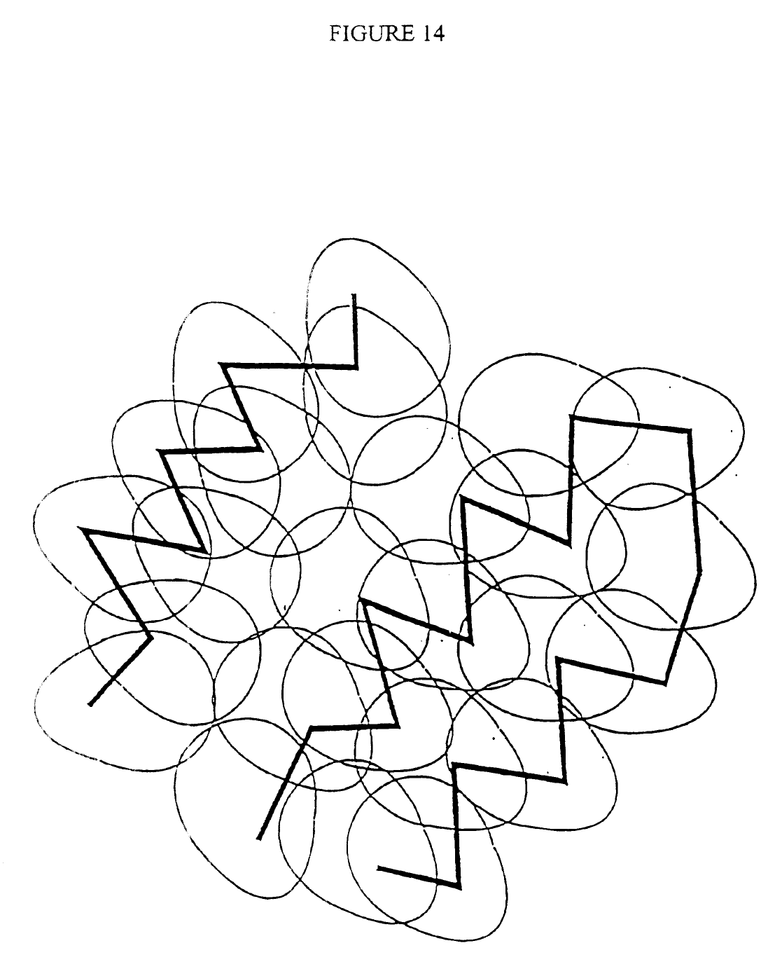

The template (more precisely the structural fragments of the template protein that correspond to the aligned residues of the probe sequence) was projected onto the underlying cubic lattice. The corresponding three-dimensional array, initially filled with zeros, was then updated to store a loose trace of the template. All elements of the array that were closer than 6^{1/2} lattice units from template residues were assigned the corresponding residue numbers. When a lattice point was within a distance of 6^{1/2} from two or more residues, the number of the closest residue was assigned to the corresponding element of the occupancy array. In the direction towards the center of mass of the template, the cut-off distance for creating the template “tube” was equal to 14^{1/2} instead of the 6^{1/2} value in the other direction. This filled in most of the volume occupied by the template structure. FIG. 14 schematically shows such tubes surrounding the aligned fragments of the template chain (in solid lines). To illustrate the above-mentioned different width of the tube in the directions towards the center (versus the outside) of the template structure, the blobs forming tube were shifted towards the center of mass of the template. This facilitated the close packing of the query (target) chain that wanders within the tube.

As described in the previous section, the starting model was placed into the template tube. The initial alignment provided an equivalence list between the template and target residue indices. This was called “the old assignment” in contrast to the “new assignment” which was generated by the program. Both the old and the new assignments were then evaluated and updated in the following way:

a) At very beginning of the simulation process, the old assignment (the original alignment) was copied into the new assignment list. The entries of these lists identify the tube compartments and the equivalent residues of the template chain. Then, all residues for which the total number of long distance (i−j>4) contacts for a three-residue fragment (with the residue of interest as a central one) was smaller than 2 become non assigned both in the old and new assignment lists. This erased those template fragments that did not interact with the rest of model protein. Thus, “non compact” fragments of the template are ignored.

b) The new assignment was then modified when, for a steric reason (or due to local stiffness), the initial query chain residue simultaneously satisfied the following two criteria: (i) the bead of the query chain was farther away than 5 lattice units from the corresponding template residue of the original equivalence assignment (“old assignment”), (ii) the position of the query chain residue (the central point of the excluded volume cluster) coincided with a lattice point that is assigned to any other template residue. The number read from the appropriate element (occupied by the lattice chain) of the occupancy array that corresponded to the bead coordinates became the updated entry of the new equivalence list.

c) For all residues of the starting query chain that were farther away than 9 lattice units from the equivalent (according to the old assignment) template residues, both old and new assignments were erased. These residues also became non-assigned. All allowed updates of the old assignments could only remove some entries from the equivalence list, which meant that some part of the threading alignment was erased. The new assignments were dynamic (due to the updates described in b), and they had the character of a structural superposition, which was not sequential in many places.

This updated pair of assignments of the query chain residues to the template defined a flexible tube around the template chain. To keep the moving query chain in the neighborhood of the template, a set of biases was introduced. First, the model chain was kept in the broad vicinity of the original template (according to the updated old assignment list) by

E_{temp,o}=Σδₒ(i)fᵣmax{0,(|rᵢ-rₒᵢ|−9)} (13)

where f, was a constant (equal to 1 kT in all simulations), rᵢ was the position of the query chain, rₒᵢ was the position of the template and δₒ(i) was equal to 1 (0) when the residue i was assigned (non assigned) according the old alignment.

Then, the residues of the query chain were similarly bonded to the template residues in the new assignment by

E_{temp,n}=Σδₙ(i)fᵣmax{0,(|rᵢ-rₙᵢ|−R_{t.})} (14)

where rₙᵢ was the position of the initial template according to the new assignment and δₙ(i) was equal to 1 (0) when the residue i was assigned (non-assigned) according the new assignment. The constant R_{t.} was equal to 7 (4) when residue i occupied any point of the template tube (the residue was outside the tube, i.e., the occupancy array at position rᵢ had value 0).

Additional restraints were the following:

E_{tube}=−E^{rep}Σ{δₒ(i)δ₃(i)+δₙ(i)+δₙ(i)δ_{c}(i)}

where δ₃(i) was equal to 1 when the residue i of the query chain was at a distance smaller than 3 lattice units from the template according to the old assignment, otherwise δ₃(i) equaled 0. The second component, δₜ(i), was equal to 1 (0) when the residue was anywhere in the template tube (is outside). δ_{c}(i) was equal to 1 for a “quasi-continuous” alignment on the tube, i.e., when {al(i−1)+al(i+1)}/2−al(i)<2, where al(i) was the value of occupancy array in the tube for residue i of the query chain, otherwise δ_{c}(i) equaled 0.

A small energy reward was also provided when the secondary structure of the query chain was consistent with the template structure. For all residues that were in extended or helical states (as defined in the loose conformational definition used for the generic short range potentials) and that were in agreement with the secondary structure read from the corresponding fragments of the template protein, the system was stabilized by an energy equal to −ε_{gen}.

With the above restraints, the system only paid a small energetic penalty for moving along the template tube (shifts in the alignment with possible lateral adjustment); however, the penalty was large for escaping from the loosely defined volume occupied by the template. For instance, it was possible that continuous fragments of the original alignments permute (this cannot be called an alignment in the conventional sense) by swapping their original tube compartments. This only occurred when the potential strongly favored such a rearrangement of the topology. The two assignments, carried out by the algorithm, played a different role. The “old” one bonded the model chain to the broad vicinity of the threading-based template. The “new” dynamic assignment was a compromise between the template restraints and packing requirements of the model chain.

Summary of the Threading Model Refinement Protocol

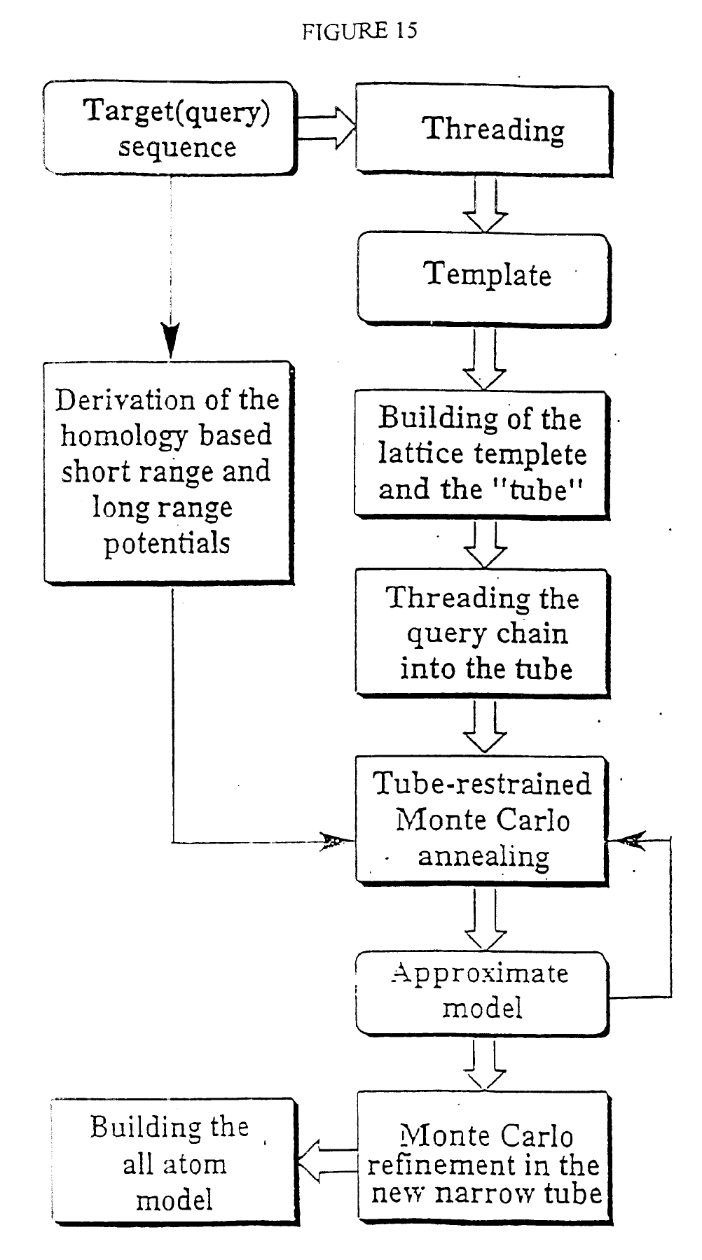

The entire model building procedure is illustrated in a flow-chart (FIG. 15) and can be outlined as follows:

a) generate the threading alignment between the query sequence and the template structure;

b) derive the sequence similarity based short and long-range pairwise potentials. The structures of proteins homologous to the query sequence are excised from the structural database; however, multiple alignments with the homologous sequences of unknown structures were used in the potential derivation procedures;

c) build the starting continuous model chain onto the lattice projected template structure;

d) build the tube around the aligned fragments of the template structure. Then, perform the first state of Monte Carlo refinement, where simulated annealing is done over a temperature range of 2-1. Since the Monte Carlo algorithm corrects unlike fragments of the alignment, the simulated annealing run is repeated two times. Subsequent runs have no systematic effect on the obtained models;

e) refinement of the structure. The model obtained from the above simulations is assumed to be the new template, with a full length, complete self-alignment. The distance restraints from the new template are narrowed to 4 lattice units, and simulated annealing is performed over a narrower temperature range (1.5 to 1.0);

f) selection of the lowest energy structures, by short isothermal simulations at T=1, followed by building the all-atom models using MODELLER.²⁴

Results

Test Proteins, Templates and Starting Alignments

Twelve pairs of target/template proteins of very low sequence similarity were selected for the present study. These proteins belong to various classes of small globular proteins, with the selected set being rather representative. As described in the Methods section, the relative scaling of the various potentials of the model force field has been adjusted in a series of ab initio folding simulations on several (different from described here) small proteins. For the tuning of the template restraint contribution, three proteins, 2pcy, 256b and 1hom, were selected. These proteins belong to rather different structural classes: 2pcy is a quite irregular β-type protein with a very poor initial threading-based model, when the 2azaA template is used. 256b is a compact, four-helix bundle, where the original alignment appears to be quite good; however, the template and target structures have a different packing of helices that needs to be significantly readjusted to obtain a reasonable model. A very different example is 1hom. Here, the target fold is not very compact, and it is important to see if the proposed procedure can handle such small open structures. All proteins were subject to the previously described model building/refinement procedure. The list of these proteins is given in Table VIII. The threading alignments have been generated by a standard threading algorithm.¹⁵ These alignments are compiled in Table IX. Tables VIII and IX appear below.

TABLE VIII
List of target/template pairs studied in this work

| Target Protein | | | Template Protein | | |
|---|---|---|---|---|---|
| PDB Code | Name | Length | PDB Code | Name | Length |
| 1aba_ | Glutaredoxin | 87 | 1ego_ | Glutaredoxin | 85 |
| 1bbhA | Cytochrome C | 131 | 2ccy_ | Cytochrome C | 127 |
| 1cewI | Cystatin | 108 | 1molA | Monellin | 94 |
| 1hom_ | Antennapedia | 68 | 11fb_ | Transcription | 77 |
| | protein | | | factor | |
| 1stfI | Papain | 98 | 1molA | Monellin | 94 |
| 1tlk_ | Telokin | 103 | 2rhe_ | Immunoglobulin | 114 |
| 256bA | Cytochrome C | 106 | 1bbh_ | Cytochrome C | 131 |
| 2azaA | Azurin | 129 | 1paz_ | Pseudoazufrin | 120 |
| 2pcy_ | Plastocyanin | 99 | 2azaA | Azurin | 129 |
| 2sarA | Ribonuclease | 96 | 9rnt_ | Ribonuclease | 104 |
| 3cd4_ | T-cell surface | 178 | 2rhe_ | Immunoglobulin | 114 |
| | glycoprotein | | | | |
| 5fdl_ | Ferrodoxin | 106 | 2fxd_ | Ferrodoxin | 81 |

TABLE IX

Starting alignments employed in model building

| | |
|---|---|
| 1aba_: | +TL,6----MPEKVYGDSNIHKCGPCDNAKRLL----TVKKQPFEFINI-MPEKGVFDDEKIAE--LLTKLGRDTQIG |
| 1ego_: | MQTV---IFGRS---GCPYCVRAKDLAEKLSN-ERDDFQYQYVDIRAEGI-TKEDLQQKA-----GKPVE-- |
| 1aba_: | LTMPQVFAPDGSHIGGFDQLREYFK----- |
| 1ego_: | TVPQIFV-DQQHIGGYTDFAAWVKENLDA |
| 1bbhA: | --AGLSPEEQIETR-----QAGYEFMG---WNMGKIKANLEGEYNAAQVEAAANVIAAIANSGMGALYGPG-TD |
| 2ccyA: | QS---KPEDLLKLRQGLMQTLKSQWVPIAGFAAGKADLPADAAQRAENMAMVAKLAPIGWAKGTEAL-PNG-- |
| 1bbhA: | KNVGDVKTRVKPEFFQN--MEDVGKIAREFVGAANTLAEVAATGEAEAVKTAFGDVGAACKSCHEKYRAK |
| 2ccyA: | --------ETKPEAFGSKS-AEFLEGWKALATESTKLAAAAKAGP-DALKAQAAATGKVCKACHEEFKQD |
| 1ccwI: | -----GAPVPVDE-NDEGLQRALQFAM-AEYNRASNDKYS-SRVVRVISA------KRQLVSGIK-YILQV-- |
| 1molA: | GEWEI---IDIGPF----TQNLGKFAVDEENKIGQYGRLTFNKVIRPCMKKTIYENERE----IKGYEYQLYV |
| 1cewi: | EIGRTTCPKSSGDLQSCEF----HDEPEMAKYTTCTFVVYSIP---WLNQIKLLESKCQ-- |
| 1molA: | Y---------ASDKLFRADISEY-------KTRGRKLLRFNGPV------------PPP |
| 1hom_: | MRKRGRQTYTRYQTLEL----EKEFHFNRYLTRRRR-----------IEIAHALC-----------------L |
| 1lfb_: | -------------RFKWGPAS-QQI-LFQAYERQKNPSKEERETLVE-ECNRAECZQRGVSPSQAQGLGSNLV |
| 1hom_: | TERQIKIWFQNRRMKWKKENKTKGEPG |
| 1lfb_: | TEVRVYNWFANR---RKEEAFRH---- |
| 2pcy_: | ---IDVLLGADDGSLAFVPSEFSISPGEKIVEK-----------NNAGFPHNIVFDEDSIPSGVDASKISMSE |
| 2azaA: | AQC-EATIESND-AMQYDLKEMVVDKSCK-QFTVHLKHVGKMAKSAMG--HNWVLTKEADKEGVATDGMNAGL |
| 2pcy: | EDLLNA--------------KGETFEVAL-----SNKGEYSFY-CSPHQGAGMVGKVTVN-- |
| 2azaA: | AQDYVKAGDTRVIAHTKVIGGGESDSVTFDVSKLTPGEAYAYFCSFPGHWAMMKGTLKL-SN |
| 1stfI: | -MMSGAPSATQPATAETQ-HIADQV-RSQLEE-KYNKK-FPV-FKAVSFK-----SQVVAGTNYFIKVHVGDE |
| 1molA: | G-------EWEIIDIGPFTQNLGKFAVDEENKIGQYGRLTFNKVIRPCMKKTIYENEREIKG-YEYQLYVYAS |
| 1stfI: | DFVHLRVFQSLPHENKPLTLSNYQTNKAKHDELTYF |
| 1molA: | DKLFRADI-SEDYKTRGRKLLRF---NGPVPPP--- |
| 1tlk: | VAEEKPHVKPYFTKTILDMD------VVEGSAARFDCKVEGY---------P------DPEVMWFKDDNPVKES |
| 2rhe_: | ---------------ESVLTQPPSASGT--PGQRVTISCTGSATDIGSNSVIWYQQVPGKAPKLLIYYNDLLPSG |
| 1tlk_: | -RHFQIDYDEEGNCSLTISEVCGDDDAKYTCKAVNSLGEAT-----CTAELLVETM-- |
| 2rhe: | VSDRFSASKSGTSASLAISGLESEDEADYYCAAWNDSLDEPGFGGG---TKLTVLGQPK- |
| 256bA: | --ADLEDNMETLNDNLKV------------JEKADNAAQVKDALTKMRAAALDAQKAT-PPKLEDKSPD-S--- |
| 1bbhA: | AGLSPEEQIETRQAGYEFMGWNMGKIKANLEGEYNAAQVEAAANVIAAIANSGMGALYGPGTDKNVGDVKTRV |
| 256bA: | -PEMKDFRHGFDIL------VGQIDDALKLANBGKVKEAQAAAEQLKTTRNAYHQKYR-- |
| 1bbhA: | KPEF--FQNMEDVGKIAREFVGAANTLAEVAATGEAEAVKTAFGDVGAACKSCHEKYRAK |
| 2azaA: | AQCEATIESNDAMQYDLKEMVVDKSCKQFTVHLKHVGKMAKSAM-----GHNWVLTKEADKEG-----VATDG |
| 1paz_: | ---------------------------ENIEVHM--LNKGAEGAMVFEPA---YI---KANPGDTVTFIPVDKG |
| 2azaA: | MNAGLAQDYVKAGDTRV-IAHTKVIGGGESDSVTFDVSKLTPGEAYAYFCS-FPGHWA--MMKGTLKLSN--- |
| 1paz_: | HNVESIKDMIPEGAEKFK--------SKINENYVLTVTQ--PG-AYLVKCTP---HYAMGMI-ALIAVGDSPA |
| 2azaA: | ----------------------- |
| 1paz_: | NLDQIVSAKKPKIVQERLEKVIA |
| 2sarA: | --------------DVSGTVCLSALPPEATDTLNLIAS-DGPFPYSQDGV----VFQNRESVLPTQSYGYYHEY |
| 9rnt_: | ACDYTCGSNCYSS------------SDVSTAQAAGYKL---HEDGETVGSNSY-PHKYNNYEGFDFSVSSPYY |
| 2sarA: | TV----------ITPGARTRGTRRIICGEATQEDYYTGDHYATFS---LIDQTC- |
| 9rnt_: | EWPILSSGDVY--SGGSPGADRVVFN---ENNQLAGVITHTGASGNN--FVECT- |
| 3cd4_: | K-----KVVLGKKGDTVELTCTASQKKS----IQFHWK---NSNQIKILGNQGSFLTKGPSKLNDRAD-SRRSL |
| 2rhe_: | ESVLTQPPSASGTPGQRVTISCTGSATDIGSNSVIWYQQVPGKAPKLLIYY---NDLLPSGVSDRFSAS---- |
| 3cd4_: | WDQGNFPLIIKNLK----IEDSDTYICEVEDQKEEVQLLVFGLTANSDTHLLQGQSLTLTLESPPGSSPSVQC |
| 2rhe_: | KSGTSASLAISGLESEDEADYY---CAAWNDSLDEPG----------------FGGGTKLTVLGQPK------ |
| 3cd4_: | RSPRGKNIQGGKTLSVSQLELQDSGTWTCTVLQNQKKVEFKIDVVLA |
| 2rhe_: | ----------------------------------------------- |
| 5fdl_: | AFVVTDNCIKCKYTDCVEVCPVDCFYEGPNFLVIHPDEC-IDCALCEPECP-AQAIFSEDEVPEDM-QEFIQL |
| 2fxd_: | ----------------------------PKYTIVDKETCI------ACGACGAAAPDIYDYDEDGIAYVTLDDN |
| 5fdl_: | NAE----LAEVWPNITE-KKDPLPDAEDWGVKGKLQHLER--- |
| 2fxd_: | QGIVEVP-DILIDDMMDA--FEGCPTDSIKVADEPFDGDPNKFE |

Compilation of the Modeling Results

Due to its stochastic character, the entire simulation procedure was repeated several times for each case of the target template chains. The resulting structures were then subject to a refinement run. Namely, the algorithm employed in the first stage of the Monte Carlo modeling (starting from the initial, “old” threading-based alignment and performing all the updates of the alignment described in the “implementation of the template restraints” section) was used in short isothermal runs at low (T=1) temperature, with the final structure obtained at the end of the first state of Monte Carlo used as input. At this temperature, the model did not change any of its global features, rather only local fluctuations were seen. The average conformational energy, which included the intrinsic force field of the model and the effect of template restraints, was then used to select the “best” structure. The model had quite a strong RMSD versus energy correlation far from the native state. Closer to native state, the two quantities became uncorrected or the correlation was weak, depending on the case. It should be pointed that out that this refers to the entire force field (intrinsic and the template biases). A quite different situation was observed for just the intrinsic force field; this was the strongest correlation of RMSD versus energy near the native structure (unpublished results). Since all the models were, at best, of moderate resolution, this criterion was no better than the one based on the total energy. The lowest average (total) energy conformation from these short isothermal runs was selected for further consideration. For example, in the case of 1tlk, a structure that had a RMSD of 4.4 Å from native was selected, while several simulations resulted in structures about 3 Å from native.

Tables X and XI, below, contain a compilation of the simulation results.

TABLE X
Alpha carbon RMSD from native for models built from the initial
threading alignments and refined by lattice simulations.

| Target Protein | Threading + MODELLER | SICHO + MODELLER |
|---|---|---|
| 1aba_ | 4.43 | 4.86 |
| 1bbhA | 6.77 | 6.82 |
| 1cewI | 14.96 | 14.38 |
| 1hom_ | 7.82 | 3.70 |
| 1stfI | 6.40 | 5.95 |
| 1tIk_ | 7.23 | 4.17 |
| 256bA | 6.09 | 4.36 |
| 2azaA | 21.95 | 10.77 |
| 2pcy_ | 6.56 | 4.41 |
| 2sarA | 10.28 | 7.83 |
| 3cd4_ | 6.74 | 6.39 |
| 5fdl_ | 25.67 | 12.40 |
| Note: The threading + MODELLER models use the threading alignments (for the aligned residues) as the target for all-atom reconstruction. SICHO models are the reduced lattice models obtained by the method | | |
| # described in this work.-The final all-atom model is also built by MODELLER using as a target the lattice model alpha carbon positions estimated from the SICHO lattice model. The values of the RMSD for alpha-carbon | | |
| # traces (in Å) are given for the structured parts of the target molecules (1hom_: residues 7-59, Itlk_: residues 9-103, 3cd4_: residues 1-97 i.e., the first domain). | | |

TABLE XI
Alpha carbon RMSD (in Å) from native for models built by MODELLER
and by lattice simulations SICHO for the aligned residues only.

| Target | Starting | MODELLER | SICHO | |
|---|---|---|---|---|
| Protein | RMSD | RMSD | RMSD | Length |
| 1abs_ | 4.37 | 3.89 | 4.40 | 69 |
| 1bbhA | 7.03 | 6.35 | 6.69 | 116 |
| 1cewI | 12.88 | 12.37 | 10.74 | 69 |
| 1hom_ | 5.59 | 5.34 | 3.45 | 40 |
| 1stfI | 7.05 | 6.04 | 4.73 | 83 |
| 1tlk_ | 7.88 | 7.15 | 3.94 | 86 |
| 256bA | 6.92 | 6.06 | 4.37 | 104 |
| 2azaA | 11.04 | 13.53 | 9.94 | 80 |
| 2pcy_ | 7.64 | 6.65 | 4.36 | 94 |
| 2sarA | 8.28 | 8.07 | 7.60 | 73 |
| 3cd4_ | 5.72 | 5.56 | 5.22 | 82 |
| 5fdl_ | 12.38 | 12.18 | 11.94 | 69 |
| Note: The starting RMSD is for the set of threading-aligned residues of the template from the equivalent native target coordinates. The MODELLER models use the threading alignment. | | | | |
| # and an all-atom target SiCHO models are the all-atom models built by MODELLER using the lattice models (only Cα) as a target. The length of the alignment. is given in the last column. | | | | |

In Table X, the Cα RMSD from the native are compared for two kinds of molecular models. The first were generated using the initial threading template followed by automated modeling using MODELLER. While this homology modeling tool is not intended to be used in such a way, some means was needed for comparing the two automated methods of model building from poor initial data. The second set of RMSD values is for the present lattice models, which for a convenient comparison were converted into the full-atom models also via an automatic application of MODELLER (with lattice models of the Cα backbones used as templates). As indicated, the most significant improvement of the model quality occurs when the threading alignment produces a rather poor but not nonsensical initial model (compare Tables X and XI). As shown in Table XI, for small globular proteins, such threading-based models have an RMSD in the range of 6-8 Å from native (over the aligned fragments). When the threading models are poor, e.g., for 1cewI or 2azaA, the improvement is rather small. At the other extreme are those cases when the alignment is good, and the resulting RMSD relatively small. Here also, the changes are small because the models are already good. Importantly, the procedure essentially does no harm to these models; thus, it can be applied to all situations with impunity. In summary, in 6 of 9 tests cases (in 9 of 12 including the three proteins employed in the model turning procedure), the models generated by the invention give lower values of RMSD over the set of aligned residues. In the three remaining cases, the changes in RMSD were insignificant (essentially in the range of the statistical fluctuations). In five cases, qualitative improvements were observed (for the aligned residues as well as for entire models; compare data given in Table 4): from 5.6 Å to 3.5 Å for 1hom, from 7.1 Å to 4.7 Å for 1stfl, from 7.9 Å to 3.9 Å for 1 tlk, from 6.9 Å to 4.4 Å for 256b or from 6.6 Å to 4.4 Å for 2pcy. These numbers were for the initial threading and final lattice (refined with MODELLER) models, respectively. It should be noted that the MODELLER refinement of the final lattice models changed their RMSD very little (in the range of 0.2 Å), while the improvement of the initial threading models by the application of MODELLER was more noticeable.

It is very interesting to see how the proposed procedure deals with the non-aligned part of the model. Comparison of the RMSD values for the aligned parts (Table XI) and for the entire structured parts (Table X) of the model reveals that the algorithm built rather reasonable models of that entire structure, provided there was a well defined fragment of good geometrical fidelity in the original alignment. Again, in all but two cases, the present method lead to more accurate models. For both the aligned part of the molecules and for entire chains (Table 4), good models were generated in about half of the studied cases (including all three proteins used in the model turning procedure). In the remaining cases, models were seen that were marginally improved, as for 3cd4, or that remained rather poor final models, as for 2azaA or 5fdl; this was true despite an RMSD decrease of more than 10 Å, as compared to models generated automatically by MODELLER from the initial threading results.

Discussion

Means of the Model Improvement

There are several ways in which the invention changes the protein model from the original fragmentary threading model. First, non-aligned parts (e.g., loops) are added and readjusted according, to packing requirements and the preferences encoded in the force field. Then, the entire chain has some freedom of movement within the template tube without any changes in its template-target sequence assignment. Furthermore, parts of the chin can slide along the tube, thereby allowing for a quite substantial modification of the initial alignment and, consequently, the resulting structure. Finally, the aligned fragments can leave the tube in a lateral direction. These segments can enter a different part of the template tube or remain outside of it. Such motions of the model chin could result in a large change of the structures, or even a change of the fold topology. The last, rather radical mode of the model rearrangements happened in several cases. In other words, the most effective way of model improvement was by neglecting a part of the threading alignment, even at the expense of various template-related energetical penalties. Interestingly, those sections of the threading-based model that were consistent with the target structure underwent only very minor changes in all cases, and the alignment remained unchanged. As discussed below, this observation may help identify those models that should be good quality from those for which improvement of the starting threading model is not satisfactory.

Below, for three selected cases, more detailed specific rearrangements of the initial threading models that took place during the Monte Carlo simulations are presented.

2pcy

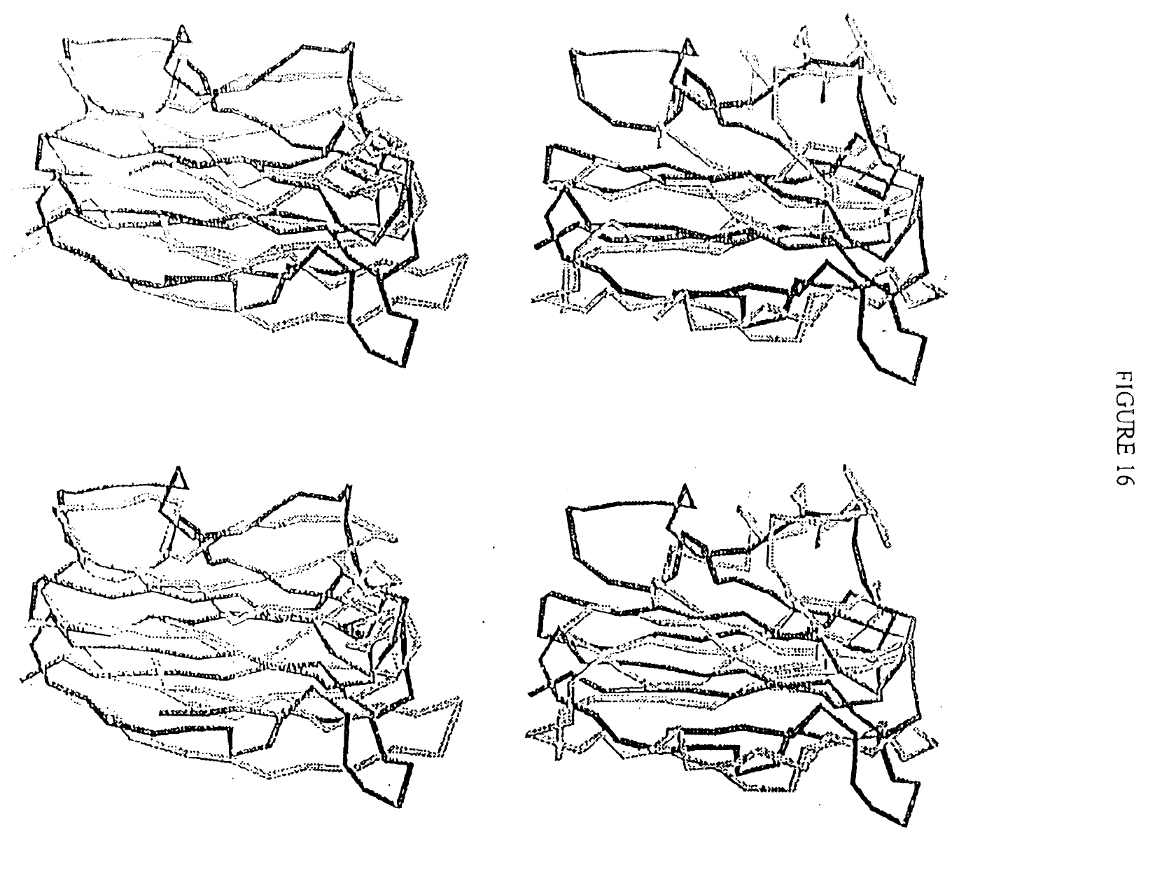

The threading alignment of the 2pcy sequence on 2azaA covered a substantial part of the sequence. There are gaps of substantial length. As a result, the threading model had the wrong topology, and two-edge strands of the eight-member β-barrel (one in each of the two P-sheets) were located in the wrong sheets. This was the reason for the resulting 7.6 Å RMSD from native for the models built solely from the threading alignment. During the simulations, the three C-terminal strands remained almost unchanged. Similarly, the three N-terminal strands underwent only small adjustments; however, in several models, one or two strands slid along the tube by a distance that sometimes changed the original alignment by one or two positions. The central fragment of the model chin (two putative irregular strands, with a couple of short helices breaking these strands) was responsible for the large RMSD in the initial model. The algorithm erased most of the template-target assignments in this part of molecule. Partly this occurred because of the compactness criterion; several residues did not have any long-range contacts in the threading model. During the simulated annealing process, residues 30-37 (small differences in the extension of this fragment can be seen between the particular runs) switched their sheet assignment, and joined the tube fragment associated with one of the C-terminal β-strands, the third one from the C-terminus. This was seen in the final “new assignment”, or pseudo-alignment. At the same time, the second strand (completely helical in the threading model) moved to the second sheet, and the long helix breaks and becomes distorted, as actually occurs in 2pcy's native structure. Most of the displaced residues joined the tube fragments generated by various secondary structural element of the template, but only a few maintained their original assignments to the template tube. This way the internal force field of the lattice model overrode the target interactions, significantly correcting the threading model. The initial model and the final model are compared with the native structure in FIG. 16, where stereo alpha-carbon traces are displayed in their best mutual superposition, using the MOLMOL²⁰ drawing program.

256bA

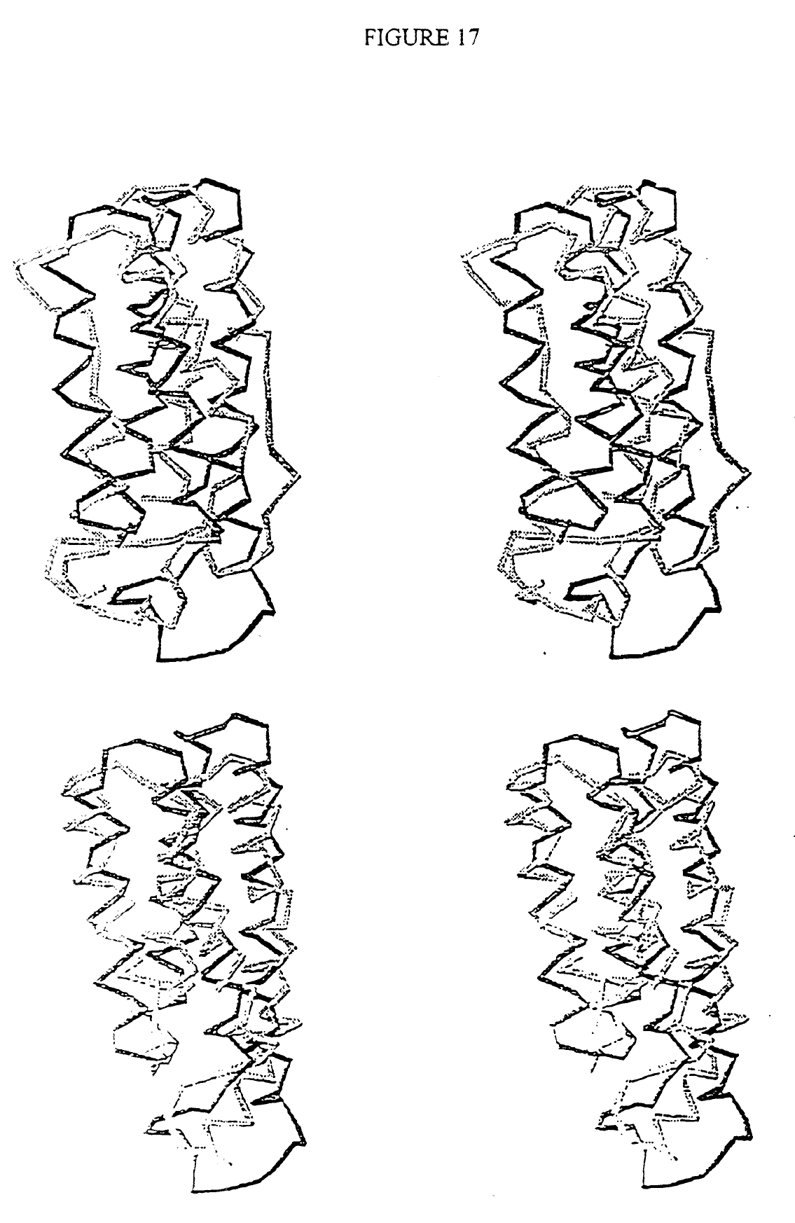

With regard to this protein, there was a four-helix bundle and the threading alignment had a few gaps. The template structure was very similar to the target, but the threading model was not very good. During the simulations, most of the C-terminal helical hairpin remained almost unchanged, except for the loop region that was very mobile. the third (first helix of the C-terminal hairpin) helix of the model was the most stable. The N-terminal hairpin underwent a large-scale rearrangement. The second helix underwent a rotation that changed its packing angle with respect to the remainder of the molecule. As a result, the end of this helix moved by about 7 Å in a lateral direction, while the beginning of this helix stayed close to its original position. The largest changes were observed for the first N-terminal helix. It moved along the tube, changing assignment indices by several residues (up to 8); a lateral adjustment took place as well. the initial model and the final model (superimposed onto the native structure) are compared in FIG. 17. The helical regions of the final model are very close to the native structure; the largest errors that account for most of the structure errors are in the central turn/loop region.

1tlk

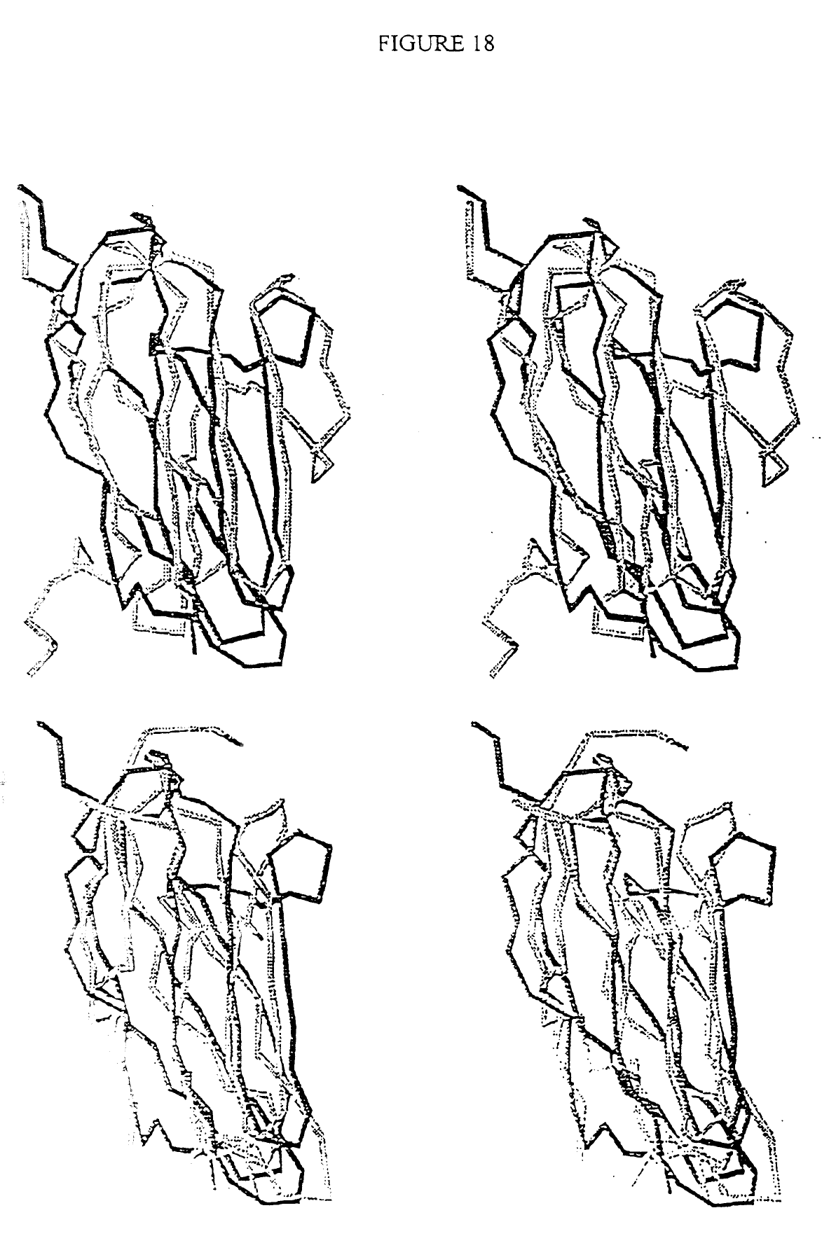

Telokin is a quite regular β-protein. Again, due to gaps and insertions, the threading model for it produced a wrong topology. During the simulations, one of the β-strands from the original model left in the initial assignment and stuck to the tube of a strand from the opposite sheet. Two β-strands that were not in the threading model (lack of the alignment assignments) were built in the simulated annealing procedure, and they joined tubes associated with existing strands. The entire structure, except for the last β-terminal β-strand that remained essentially unchanged, rearranged substantially. Mostly lateral (orthogonal to the local direction of the template tube) displacements occurred in the range of 6 Å for about half of all the residues. As a result, the model improved its RMSD by almost 4 Å. The initial model and the final model (superimposed onto the native structure) are compared in FIG. 18.

How to Identify Good Models

As mentioned above, the instant invention generates low to moderate resolution models of correct topology in those cases when the initial threading-based alignment leads to at least a partially correct structure, i.e., where a part of the identified template is close to the target structure. How to (a priori) distinguish a good (threading-based) alignment from a poor one is a non-trivial question. Unfortunately, there is not yet a general solution to this problem.

The intrinsic force field of the reduced model correctly identifies the native structure (the lattice protection) as the lowest energy conformation when compared with the models generated by MODELLER from the initial threading alignments. The models obtained in the lattice homology modeling are described herein. In all cases except one (b 1bbhA, where MODELLER gave a slightly better result than the present method), the energy of the models built by the present method is significantly lower than other worse models (including these built by automatic use of MODELLER). While interesting, there remains a need to be able to distinguish those target/template pairs where the final model is of reasonable quality from those cases where, despite a sometimes large improvement of the initial models, the resulting structures are still far from the native target conformation. Unfortunately, simple energetic criteria (conformational energy per residue in the final model, decrease of energy from the starting model to the final model, etc.) do not enable identification of these poor quality structures.

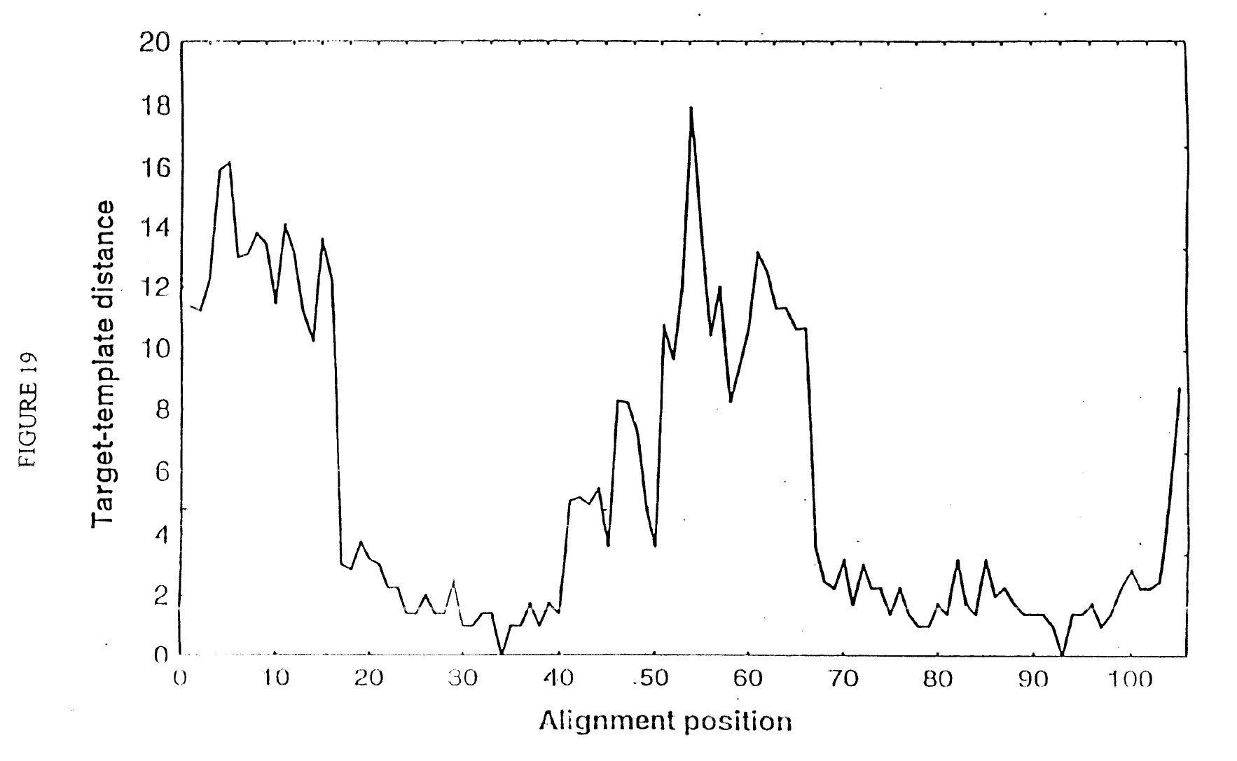

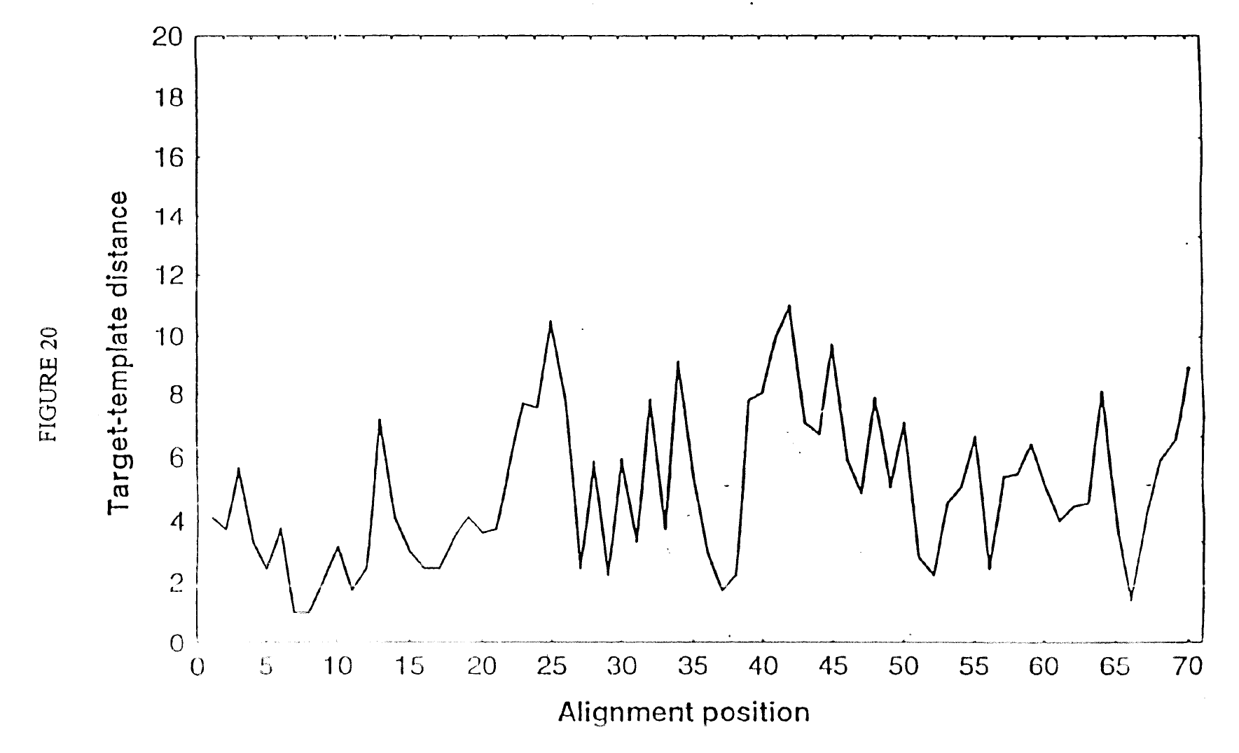

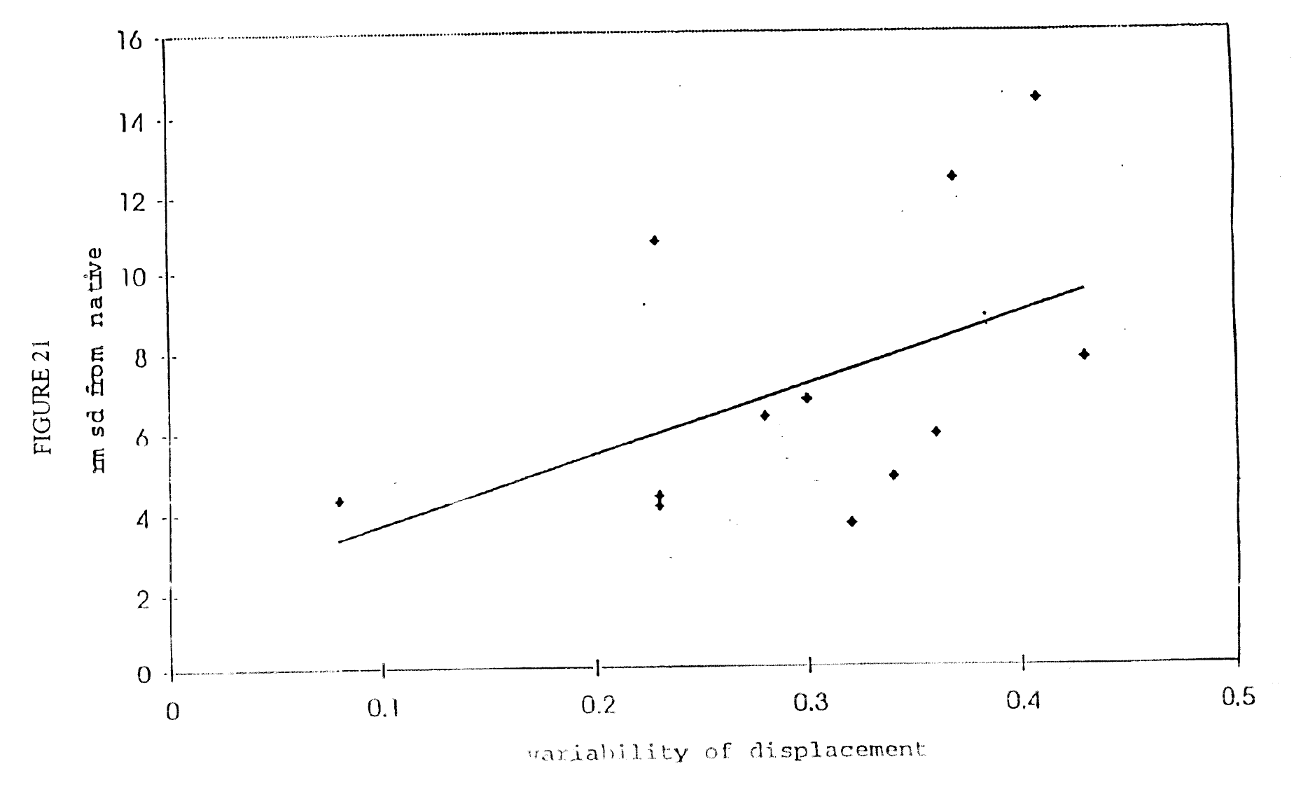

The previous section discussed how the modeling procedure of the invention improves the initial, threading-based model. This could be actually used for a qualitative identification of better models. Consider the displacement of particular residues (as a function of their position along the chain) during the entire simulation procedure. In those cases where the final model is of good quality, the plots indicate relatively well separated regions where the chain modifications were small and also indicated regions of large modifications. This is consistent with the previously mentioned characteristic behavior of “good” models, for which some ligaments of alignments are recognized by the procedure as being very good and behave as a scaffold for readjustment of the remainder of the protein. In contrast, poor models are characterized by random fluctuations of the spatial amino acid displacements along the sequence. In such cases there is no pattern. Perhaps, there is a huge energy barrier between the starting model and the better, near native models that cannot be surmounted by partial readjustment of the initial alignment. Examples of both situations are given in FIGS. 19 and 20. The lowest (and locally similar) displacement (during the modeling procedure) regions identify the regions of an optimal (or very close to optimal) alignment. While the above is not easy for a simple quantification, it still can be used as a heuristic criterion for the identification of cases where the method proposed in this work is likely to provide relatively good, low resolution models. FIG. 21 shows the plot of model accuracy (measured as the alpha carbon RMSD from native) as a function of the variability in the model chain mobility during the simulations. Unfortunately, the correlation is not very strong. Consequently, the mobility criterion has to be used with caution. Rather plots as given in FIGS. 19 and in 20 can be used to identify the best fragments of threading models. Indeed, there are very strong correlations between the lowest mobility and the best structural fidelity (to the target structure) of the model chain fragments. this may have some other applications, where assessment of the reliability of various parts of a model structure is needed.

Summary and Conclusion

In this example, the invention again was shown to be useful in predicting medium- to low-resolution protein structures based on homology or sequence-structure compatibility. Here, the initial alignment between the target and template was generated by a threading procedure. Of course, alignments also can be obtained by other means, e.g., from sequence alignments. Such templates are used to guide Monte Carlo simulations that employ a reduced protein chain representation built using pseudoatoms to represent the side chain center of mass of the various amino acid residues of a protein or protein domain. In contrast to the method of example 1, the pseudoatoms of the SICHO model used here took also took account of alpha-carbon atoms, in addition to the corresponding side chains. This alternate embodiment of the model proved capable of making large structural rearrangements that, in about a third of studied cases, lead to qualitative improvements in the initial poor models. In some other cases, despite a huge decrease in the RMSD between the model and the target native structure, the final model was still not satisfactory. The analysis of the simulation trajectories allows for the plausible identification of those cases where the final model improves qualitatively with respect to the initial, threading-based model.

The present invention is useful for large-scale protein structure and function prediction. Using the invention, it is possible to identify the biochemical function of a protein function having a model with a 5-6 Å backbone RMSD.^{7,8} Certainly, it would be much more difficult, if not impossible, to make such an identification for a model with an 8 Å Cα RMSD from native polypeptide. For example, the model of plastocyanin (2pcy) generated above had its four copper-binding residues much closer to their native position than predicted by the threading-based model. Thus, having a structural template of this active site (e.g., an FSD), the model structure can be identified with high fidelity as a copper-binding protein. The results above show that for many new or known proteins (e.g., those identified in the course of high throughput nucleic acid sequencing programs), the invention can be used to identify their function(s). The invention also complements sequence-based and threading methods, and provides a basis for improving initially poor and incomplete models. Additionally, the invention is also complementary to standard homology modeling tools, enabling homology modeling in those cases where the template is structurally very far from the target structure.

### REFERENCES

### Example 2 Only

1. Altschul, S. F., Madden, T. L., Schaefer, A. A., Zhang, J., Zhang, Z., Miller, W. & Lipman, D. J. (1997). Gapped BLAST and PHI-BLAST: a new generation of protein database search programs. Nucleic Acid Res. 25, 3389-3402.

2. Aszodi, A. & Tylor, W. R. (1996). Homology modeling by distance geometry. Folding & Design 1, 325-34.

3. Bernstein, F. C., Koetzle, T. F., Williams, G. J. B., Meyer Jr., E. F., Brice, M. D., Rogers, J. R., Kennard, O., Simanouchi, T. & Tasumi, M. (1997). The protein data band: a computer-based archival file for macromolecular structures. J Mol. Biol. 112, 535-542.

4. Binder, K. (1991). The Monte Carlo Method in Condensed Matter Physics, Institut Für Physik, Johannes Gutenberg-Universität, Mainz.

5. Bowie, J. U., Luethy, R. & Eisenberg, D. (1991). A method to identify protein sequences that fold into a known three dimensional structure. Science 253, 164-170.

6. Bryant, S. H. & Lawrence, C. E. (1993). An empirical energy function for threading protein sequence through folding motif. Proteins 16, 92-112.

7. Fetrow, J., Godzik, A. & Skolnick, J. (1998). Functional analysis of the Escherichia coli genome using the sequence-structure-function paradigm: identification of proteins exhibiting the glutaredoxin/thioredoxin disulfide oxidoreductase activity. J Mol. Biol., 282, 703-711.

8. Fetrow, J. S. & Skolnick, J. (1998). Method for prediction of protein function from sequence using the sequence to structure to function paradigm with application to glutaredoxins/thioredoxins and T₁ ribonucleases. J. Mol. Biol., 281, 949-968.

9. Godzik, A., Skolnick, J. & Kolinski, A. (1992). A topology fingerprint approach to the inverse folding problem. J. Mol. Biol, 227, 227-238.

10. Godzik, A., Skolnick, J. & Kolinski, A. (1993). Regularities in interaction patterns of globular proteins. Protein Eng. 6, 801-810.

11. Henikoff, S. & Henikoff, J. G. (1992). Amino acid substitution matrices from protein blocks. Proc. Nat'l Acad. Sci. USA 89, 10915-10919.

12. Hobohom, U., Scharf, M., Schneider, R. & Sander, C. (1992). Selection of a representative set of structures from the Brookhaven Protein Data Bank. Protein Sci. 1, 409-417.

13. Hu, W.-P., Godzik, A. & Skolnick, J. (1997). On the origin of sequence-structure specificity. How does an inverse folding approach work? Prot. Engng. 10, 317-331.

14. Jaroszewski, L., Pawlowski, K. & Godzik, A. (1998a). Multiple model approach: Exploring the limits of comparative modeling. J. Molecular Modeling.

15. Jaroszewski, L., Ryehlewski, L., Zhang, B. & Godzik, A. (1998b). Fold prediction by a hierarchy of sequence, threading, and modeling methods. Protein Sci. 7, 1431-1440.

16. Jones, D. T., Taylor, W. R. & Thornton, J. M. (1992). A new approach to protein fold recognition. Nature 358, 86-89.

17. Kolinski, A., Jaroszewski, L., Rotkiewicz, P. & Skolnick, J. (1998). An efficient Monte Carlo model of protein chains. Modeling the short-range correlations between side groups centers of mass. J. Phys. Chem. 102, 4628-4637.

18. Kolinski, A. & Skolnick, J. (1996). Lattice models of protein folding, dynamics and thermodynamics, R. G. Landes, Austin, Tex.

19. Kolinski, A. & Skolnick, J. (1998). Assembly of protein structure from sparse experimental data. An efficient Monte Carlo Model. Proteins 32, 475-94.

20. Koradi, R. (1996). MOLMOL: a program for display and analysis of macromolecular structures. J. Mol. Graph. 14, 51-55.

21. Madej, T., Gibrat, J. F. & Bryant, S. H. (1995). Threading a database of protein scores. Proteins 23, 356-369.

22. Milik, M., Kolinski, A. & Skolnick, J. (1995). Neural Network System for the Evaluation of Side Chain Packing in Protein Structures. Protein Engng. 8, 225-236.

23. Miller, R. T., Jones, D. T. & Thornton, J. M. (1996). Protein fold recognition by sequence threading tools and assessment techniques. FASEB 10, 171-178.

24. Sali, A., Overington, J. P., Johnson, M. S. & Blundell, T. L. (1990). From comparison of protein sequences and structures to protein modeling and design. TIBS 15, 235-250.

25. Skolnick, J., Kolinski, A. & Ortiz, A. R. (1997). MONSSTER: A method for folding globular proteins with a small number of distance restraints, J. Mol. Biol, 265, 271-241.

26. Wodak, S. J. & Rooman, M. J. (1993). Generating and testing protein folds. Current Opinion in Structural Biology 3, 247-259.

One skilled in the art will readily appreciate that the present invention is well adapted to carry out the objects and obtain the ends and advantages mentioned, as well as those inherent therein. SICHO, as implemented above, is exemplary and is not intended as limiting the scope of the invention described herein. It will be readily apparent to one skilled in the art that varying alterations and modifications may be made to the invention disclosed herein without departing from the scope and spirit of the invention.

All patents, patent applications, and publications mentioned in the specification are indicative of the levels of those skilled in the art to which the invention pertains, and are hereby incorporated by reference to the same extent as if each individual publication was specifically and individually indicated to be incorporated by reference.

The invention illustratively described herein suitably may be practiced in the absence of any element or elements, limitation, or limitations not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed in various embodiments, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims.

### REFERENCES

### Excluding Example 2

1. Friesner, R. A., Gunn, J. R., Computational studies of protein folding. Annu. Rev. Biophys, Biomol, Struct. 25:315-342, 1996.

2. Levitt, M., Protein folding. Curr. Opin. Struct. Biol. 1:224-229, 1991.

3. Anfinsen, C. B., Scheraga, H. A., Experimental and theoretical aspects of protein folding. Adv. Protein Chem. 29:205-300, 1975.

4. Smith-Brown, M. J., Kominos, D., Levy, R. M., Global folding of proteins using a limited number of distance restraints. Protein Eng. 6:605-614, 1993.

5. Skolnick, J., Kolinski, Al, Ortiz, A. R., MONSSTER: A method for folding globular proteins with a small number of distance restraints. J. Mol. Biol. 265:217-241, 1997.

7. Kaptein, R., Boelena, R., Scheek, R. M., van Gunsteren, W. F., Protein structures from MNR. Biochemistry 27:5389-5395, 1988.

8. Gronenborn, A. M., Clore, G. M., Where is NMR taking us? Proteins 19:273-276, 1994.

9. Braun, W., Go, N. Calculation of protein conformations by proton-proton distance constraints. A new efficient algorithm. J. Mol. Biol. 186:611-626, 1985.

10. Havel, T. F., Wuthrich, K. An evaluation of the combined use of nuclear magnetic resonance and distance geometry for the determination of protein conformation in solution. J. Mol. Biol. 182:281-294, 1985.

11. Havel, T. F. An evaluation of computational strategies for use in the determination of protein structures from distance constraints obtained by nuclear magnetic resonance. Prog. Biophys. Mol, Biol. 56:43-78, 1991.

12. Mumenthaler, C., Braun, W. Automated assignment of simulated experimental NOESY spectra of protein from back littering and self-correcting distance geometry. J. Mol. Biol. 254:463-480, 1995.

13. Guentert, P., Braun, W., Wuthrich, K. Efficient computation of three-dimensional protein structures in solution from nuclear magnetic resonance data using the program DINA and the supporting programs CALIBA, HABAS and GLOMSA. J. Mol. Biol. 217:517-530, 1991.

14. Bernstein, F. C., Koetzle, T. F., Williams, G. J. B., Meyer Jr., E. F., Brice, M. D., Rodgers, J. R., Kennard, O., Simanouchi, T., Tasumi, M. The protein data bank: a computer-based archival file for macromolecular structures. J. Mol. Biol. 112:535-542, 1977.

15. Kolinski, A., Skolnick, J. Monte Carlo simulations of protein folding. I. Lattice model and interaction scheme. Proteins 18:338-352, 1994.

16. Kolinski, A., Skolnick, J. “Lattice Models of Protein Folding, Dynamics and Thermodynamics.” Austin, Tex.: R. G. Landes Co., 1996.

17. Kolinski, A., Skolnick, J. Parameters of statistical potentials. Available by ftp from public directory scripps/edu(pub/andr/side_only/*). 1997.

18. Godzik, A., Skolnick, J., Kolinski, A. Regularities in interaction patterns of globular proteins. Protein Eng. 6:801-810, 1993.

19. Kyte, J., Doolittle, R. F. A simple method for displaying the hydrophatic character of protein. J. Mol. Biol, 157:105-132, 1982.

20. Skolnick, J., Jaroszewski, L., Kolinski, A., Godzik, A. Derivation and testing of pair potentials for protein folding, when is the quasichemical approximation correct? Protein Sci. 6:676-688, 1997.

21. Kolinski, A., Godzik, A., Skolnick, J. A general method for the prediction of the three dimensional structure and folding pathway of globular proteins. Application to designed helical proteins. J. Chem Phys. 98:7420-7433, 1933.

22. de Gennes, P. G., “Scaling Concepts in Polymer Physics.” Ithaca, N.Y.; Cornell University Press, 1979.

23. Kolinski, A., Skolnick, J., Determinants of secondary structure f polypeptide chains: Interplay between short range and burial interactions. J. Chem. Phys. 107:953-964, 1997.

24. Eisenberg, D., McLauchlan, A. D., Solvation energy in protein folding and binding. Nature 319:199-203, 1986.

25. Godzik, A., Kolinski, A., Skolnick, J., Are proteins ideal mixtures of amino acids? Analysis of energy parameter sets. Protein Sci. 4:2107-2117, 1995.

26. Godzik, A., Knowledge-based potential for protein folding: What can we learn from known structures? Curr. Biol, 4:363-366, 1996.

27. Kolinski, A., Jaroszewski, L., Rotkiewicz, P., Skolnick, J., An efficient Monte Carlo model of protein chains. Modeling the short-range correlations between side group centers of mass. J. Phys, Chem. 102:4628-4637, 1998.

28. Kolinski, A., Skolnick, J., Monte Carlo simulations of protein folding. II. Application to protein A, ROP, and crambin. Proteins 18:353-366, 1994.

29. Kolinski, A., Galazka, W., Skolnick, J., Computer design of idealized β-motifs. J. Chem. Phys. 103:10286-10297, 1995.

30. Kolinski, A., Milik, M., Rycombel, J., Skolnick, J., A reduced model of short range interactions in polypeptide chains. J. Chem. Phys. 103:4312-4323, 1995.

31. Kolinski, A., Galazka, W., Skolnick, J., On the origin of the cooperativity of protein folding. Implications from model simulations. Proteins 26:271-287, 1996.

32. Olszewski, J., Kolinski, A., Skolnick, J., Does a backwardly read protein sequence have unique native state? Protein Eng. 9:5-14, 1996.

33. Diszewski, K., Kolinski, A., Skolnick, J., Folding simulations and computer redesign of protein Å three-helix bundle motifs. Proteins 25:286-299, 1996.

34. Ortiz, A. R., Hu, W. P., Kolinski, A., Skolnick, J., A method for prediction of the tertiary structure of small proteins. J. Mol. Graph. in press.

35. Ortiz, A. R., Hu, W. P., Kolinski, A., Skolnick, J., Method for low resolution prediction of small protein tertiary structure. In: “Proceedings of the Pacific Symposium on Biocomputing '97.” Altman, R. B., Dunker, A. K., Hunter, L., Klein, T. E. (eds.), Singapore: World Scientific Pub., 1997 316-327.

36. Skolnick, J., Kolinski, A., Monte Carlo lattice dynamics and the prediction of protein folds. In: “Computer Simulations of the Biomolecular Systems. Theoretical and Experimental Studies,” van Gunsteren, W. F., Weiner, P. K., Wilkinson, A. J. (eds.). The Netherlands: ESCOM Science Pub. 395-429, 1997.

37. Kabsch, W., Sander, C., Dictionary of protein secondary structure: Pattern recognition of hydrogen-bonded and geometrical features. Biopolymers 22:2577-2637, 1983.

Binder, K., “Monte Carlo Methods in Statistical Physics.” Berlin: Springer-Verlag, 1986.

39. Skolnick, J., Kolinski, A., Protein modelling. In: “Encyclopedia of Computational Chemistry,” Schleyer, P., Kollman, P. (eds.). Sussex, England: John Wiley & Sons, in press.

40. Richardson, J., The anatomy and taxonomy of protein structure. Adv. Protein Chem. 34:167-339, 1981.

41. Gronenborn, A., Filpula, D. R., Essig, N. Z., Achari, A., Whitlow, M., Wingfield, P. T., Clore, G. M., A novel highly stable fold of the immunoglobulin binding domain of streptococcal protein. G. Science 253:657-660, 1991.

42. Koradi, R., MOLMOL: A program for display and analysis of macromolecular structures. J. Mol. Graph. 14:51-55, 1996.

43. Goebel, U., Sander, C., Schneider, R., Valencia, A., Correlated mutations and residue contacts in proteins. Proteins 18:309-317, 1994.

44. Kolinski, Method for improvement of threading models, Proteins 37:592-610, 1999.

We claim:

1. A computer-assisted method for determining a three-dimensional structure of a target amino acid sequence using a computer comprising a processor configured to receive and output data in accordance with executable code, the method comprising:
(a) generating input data for the computer comprising:
(i) inputting into the computer an alignment of a target amino acid sequence with a template amino acid sequence; and
(ii) by way of executable code, directing the processor to produce from the alignment a three dimensional reduced protein model comprised of representations of side chains of amino acid residues comprising a target protein; and
(b) outputting the three-dimensional reduced protein model to an output device or a storage device.

2. A method according to claim 1 wherein the executable code comprises instructions for:
(a) converting representations of the side chains of amino acid residues of the target protein to interaction centers connected by virtual covalent bonds, wherein each interaction center comprises a pseudoatom representing a center of mass of the side chain of the represented amino acid to which the interaction center corresponds, and wherein each interaction center, except for the interaction centers representing the amino and carboxy terminal amino acid residues of the target protein, is connected to an immediately proximal interaction center and an immediately distal interaction center via a virtual covalent bond to produce an interaction center chain; and
(b) projecting the interaction center chain onto an underlying cubic lattice to produce a projected chain of interaction centers;
(c) applying secondary constraints and/or tertiary constraints to a subset of interaction centers of the interaction center chain so as to produce a data set representing a three-dimensional model structure of the target protein.

3. A method according to claim 2 further comprising iterating steps (a)-(c), wherein in each iteration, a different set of secondary and tertiary constraints are applied to the interaction centers to produce a series of data sets representing three-dimensional model structures of the target protein, and wherein an energy computation is made for each member of the series of data sets representing the three-dimensional model structures of the target protein.

4. A method according to claim 3 further comprising selecting the member of the series of data sets representing the three-dimensional model structures of the target protein that has the lowest energy.

5. A method according to claim 4 wherein the data set representing the three-dimensional model structure of the target protein having the lowest energy is output to the data storage system to produce a stored data set.

6. A method according to claim 4 wherein the data set representing the three-dimensional model structure of the target protein having the lowest energy is output to an output device.

7. A method according to 5 wherein the stored data set is retrieved and displayed on an output device in a manner that allows the three-dimensional model structure of the target protein to be visualized.

8. A method according to claim 1 wherein the threading alignment input into the computer is retrieved from a data storage system.

9. A computer-assisted method for determining a three-dimensional structure of a target protein using a computer comprising a processor configured to receive and output data in accordance with executable code, the method comprising:
(a) generating input data for the computer comprising:
(i) inputting as a string of an identity constraint and a secondary structure constraint and/or tertiary constraints for some or all of the amino acid residues residue comprising the target protein; and
(ii) by way of executable code, directing the processor to produce from the string a three dimensional reduced protein model comprised of representations of side chains of the amino acid residues comprising the target protein; and
(b) outputting the three dimensional reduced protein model to an output device or a storage device.

10. A method according to claim 9 wherein the secondary structure constraint for each amino acid residue is selected from the group of “H” for helix, “E” for extended, and “(-)” for other structural constraints.

11. A method according to claim 9 wherein the secondary structural constraint for a subset of amino acid residues comprising the target protein is generated by a threading alignment of an amino acid sequence of the target protein.

12. A computer-assisted method for determining a three-dimensional structure of a target amino acid sequence, the method comprising inputting into the computer an alignment of a target amino acid sequence with a template amino acid sequence and calculating with the said computer one or more three-dimensional reduced protein model comprising representations of side chains of amino acid residues comprising a target protein.

13. A method according to claim 12 further comprising outputting to an output device or a storage device one or more of the three-dimensional reduced protein models.
